(19) United States
(12) Patent Application Publication
(10) Pub. No.: US 2012/0132211 A1
(43) Pub. Date: May 31, 2012

(54) MONITORING ENDOTRACHEAL INTUBATION

(75) Inventors: Avner Halperin, Ramat Gan (IL); Roman Karasik, San Martin (IL); Guy Meger, Haifa (IL)

(73) Assignee: EARLYSENSE LTD., Ramat Gan (IL)

(21) Appl. No.: 13/305,618

(22) Filed: Nov. 28, 2011

Related U.S. Application Data
(63) Continuation of application No. 12/113,680, filed on May 1, 2008.
(60) Provisional application No. 60/924,181, filed on May 2, 2007.
(60) Provisional application No. 60/924,459, filed on May 16, 2007.
(60) Provisional application No. 60/935,194, filed on Jul. 31, 2007.
(60) Provisional application No. 60/981,525, filed on Oct. 22, 2007.
(60) Provisional application No. 60/983,945, filed on Oct. 31, 2007.
(60) Provisional application No. 60/989,942, filed on Nov. 25, 2007.
(60) Provisional application No. 61/028,551, filed on Feb. 14, 2008.
(60) Provisional application No. 61/034,165, filed on Mar. 6, 2008.

(51) Int. Cl. A61M 16/04

(52) U.S. Cl. 128/207.14

(57) ABSTRACT

Apparatus and methods are provided for use during endotracheal intubation of a subject, including an output unit, and at least one sensor configured to sense motion of the subject, and generate a signal responsively thereto. A control unit is configured to detect an aspect of the intubation by analyzing a component of the signal having a frequency of less than 20 Hz, and drive the output unit to generate an output indicative of the aspect of the intubation. Other applications are also described.

### CROSS-REFERENCES TO RELATED APPLICATIONS

The present application claims the benefit of the following US provisional patent applications, all of which are assigned to the assignee of the present application and are incorporated herein by reference:

U.S. Provisional Application 60/924,181, filed May 2, 2007;

U.S. Provisional Application 60/924,459, filed May 16, 2007;

U.S. Provisional Application 60/935,194, filed Jul. 31, 2007;

U.S. Provisional Application 60/981,525, filed Oct. 22, 2007;

U.S. Provisional Application 60/983,945, filed Oct. 31, 2007;

U.S. Provisional Application 60/989,942, filed Nov. 25, 2007;

U.S. Provisional Application 61/028,551, filed Feb. 14, 2008; and

U.S. Provisional Application 61/034,165, filed Mar. 6, 2008.

The present application is related to an international patent application entitled, “MONITORING, PREDICTING AND TREATING CLINICAL EPISODES,” filed on even date herewith, which is incorporated herein and by reference.

### FIELD OF THE INVENTION

The present invention relates generally to monitoring patients and predicting and monitoring abnormal physiological conditions and treating those conditions, and specifically to methods and apparatus for predicting and monitoring abnormal physiological conditions by non-contact measurement and analysis of characteristics of physiological and/or physical parameters.

### BACKGROUND OF THE INVENTION

Chronic diseases are often expressed by episodic worsening of clinical symptoms. Preventive treatment of chronic diseases reduces the overall dosage of required medication and associated side effects, and lowers mortality and morbidity. Generally, preventive treatment should be initiated or intensified as soon as the earliest clinical symptoms are detected, in order to prevent progression and worsening of the clinical episode and to stop and reverse the pathophysiological process. Therefore, the ability to accurately monitor pre-episodic indicators increases the effectiveness of preventive treatment of chronic diseases.

Many chronic diseases cause systemic changes in vital signs, such as breathing and heartbeat patterns, through a variety of physiological mechanisms. For example, common respiratory disorders, such as asthma, chronic obstructive pulmonary disease (COPD), sleep apnea and cystic fibrosis (CF), are direct modifiers of breathing and/or heartbeat patterns. Other chronic diseases, such as diabetes, epilepsy, and certain heart conditions (e.g., congestive heart failure (CHF)), are also known to modify cardiac and breathing activity. In the case of certain heart conditions, such modifications typically occur because of pathophysiologies related to fluid retention and general cardiovascular insufficiency. Other signs such as coughing and sleep restlessness are also known to be of importance in some clinical situations.

Many chronic diseases induce systemic effects on vital signs. For example, some chronic diseases interfere with normal breathing and cardiac processes during wakefulness and sleep, causing abnormal breathing and heartbeat patterns.

Breathing and heartbeat patterns may be modified via various direct and indirect physiological mechanisms, resulting in abnormal patterns related to the cause of modification. Some respiratory diseases, such as asthma, and some heart conditions, such as CHF, are direct breathing modifiers. Other metabolic abnormalities, such as hypoglycemia and other neurological pathologies affecting autonomic nervous system activity, are indirect breathing modifiers.

Asthma is a chronic disease with no known cure. Substantial alleviation of asthma symptoms is possible via preventive therapy, such as the use of bronchodilators and anti-inflammatory agents. Asthma management is aimed at improving the quality of life of asthma patients. Asthma management presents a serious challenge to the patient and physician, as preventive therapies require constant monitoring of lung function and corresponding adaptation of medication type and dosage. However, monitoring of lung function requires sophisticated instrumentation and expertise, which are generally not available in the non-clinical or home environment. Monitoring of lung function is viewed as a major factor in determining an appropriate treatment, as well as in patient follow-up. Preferred therapies are often based on aerosol-type medications to minimize systemic side-effects. The efficacy of aerosol type therapy is highly dependent on patient compliance, which is difficult to assess and maintain, further contributing to the importance of lung-function monitoring.

Asthma episodes usually develop over a period of several days, although they may sometimes seem to appear unexpectedly. The gradual onset of the asthmatic episode provides an opportunity to start countermeasures to stop and reverse the inflammatory process. Early treatment at the pre-episode stage may reduce the clinical episode manifestation considerably, and may even prevent the transition from the pre-clinical stage to a clinical episode altogether.

Two techniques are generally used for asthma monitoring. The first technique, spirometry, evaluates lung function using a spirometer, an instrument that measures the volume of air inhaled and exhaled by the lungs. Airflow dynamics are measured during a forceful, coordinated inhalation and exhalation effort by the patient into a mouthpiece connected via a tube to the spirometer. A peak-flow meter is a simpler device that is similar to the spirometer, and is used in a similar manner. The second technique evaluates lung function by measuring nitric-oxide concentration using a dedicated nitric-oxide monitor. The patient breathes into a mouthpiece connected via a tube to the monitor.

Efficient asthma management requires daily monitoring of respiratory function, which is generally impractical, particularly in non-clinical or home environments. Peak-flow meters and nitric-oxide monitors provide a general indication of the status of lung function. However, these monitoring devices have limited predictive value, and are used as during-episode markers. In addition, peak-flow meters and nitric-oxide monitors require active participation of the patient, which is difficult to obtain from many children and substantially impossible to obtain from infants.

Congestive heart failure (CHF) is a condition in which the heart is weakened and unable to circulate blood to meet the body's needs. The subsequent buildup of fluids in the legs, kidneys, and lungs characterizes the condition as congestive. The weakening may be associated with either the left, right, or both sides of the heart, with different etiologies and treatments associated with each type. In most cases, it is the left side of the heart which fails, so that it is unable to efficiently pump blood to the systemic circulation. The ensuing fluid congestion of the lungs results in changes in respiration, including alterations in rate and/or pattern, accompanied by increased difficulty in breathing and tachypnea.

Quantification of such abnormal breathing provides a basis for assessing CHF progression. For example, Cheyne-Stokes Respiration (CSR) is a breathing pattern characterized by rhythmic oscillation of tidal volume with regularly recurring periods of alternating apnea and hyperpnea. While CSR may be observed in a number of different pathologies (e.g., encephalitis, cerebral circulatory disturbances, and lesions of the bulbar center of respiration), it has also been recognized as an independent risk factor for worsening heart failure and reduced survival in patients with CHF. In CHF, CSR is associated with frequent awakening that fragments sleep, and with concomitant sympathetic activation, both of which may worsen CHF. Other abnormal breathing patterns may involve periodic breathing, prolonged expiration or inspiration, or gradual changes in respiration rate usually leading to tachypnea.

Fetal well-being is generally monitored throughout pregnancy using several sensing modalities, including ultrasonic imaging as a screening tool for genetic and developmental defects and for monitoring fetal growth, as well as fetal heartbeat monitoring using Doppler ultrasound transduction. It has been found that a healthy baby responds to activity by increased heart rate, similar to the way an adult's heart rate changes during activity and rest. Fetal heart rate typically varies between 80 and 250 heartbeats per minute, and accelerates with movement in a normal, healthy fetus. Lack of such variability has been correlated with a high incidence of fetal mortality when observed prenatally. In late stages of pregnancy, particularly in high-risk pregnancies, fetal heartbeat is commonly monitored on a regular basis to monitor fetal well-being and to identify initial signs of fetal distress, which usually result in active initiation of an emergency delivery. Current solutions to monitor fetal well-being are generally not suitable for home environments.

Obstructive sleep apnea (OSA) is a disorder in which complete or partial obstruction of the airway during sleep occurs due to a collision of the pharynx into the upper airway that blocks breathing. As a result, the patient suffers from loud snoring, oxyhemoglobin desaturations and frequent arousals. These arousals may occur hundreds of times each night but do not fully awaken the patient, who remains unaware of the loud snoring, choking, and gasping for air that are typically associated with obstructive sleep apnea. In contrast to central sleep apnea, OSA includes futile inspiratory efforts.

A pulmonary embolism is a sudden blockage in a lung artery, often caused by a deep vein thrombosis (DVT) that breaks free and travels through the bloodstream to the lung. Pulmonary embolism is a serious condition that can cause permanent damage to the affected lung, damage to other organs, and death, particularly if the clot is large or if there are many clots.

Many general hospital wards suffer from a chronic shortage of nurses, a fact which adversely affects the quality of healthcare and often results in gaps of between four and six hours between rounds to check patient vital signs. During these gaps, many patients are not monitored, with the practical effect that signs of deterioration are often not detected in a timely manner. As a result, some hospitals experience high rates of unexpected complications and even death (most often caused by respiratory or heart failure). Conventional ECG monitors require the attachment of electrodes to the patient's body and thus limit the patient's mobility and comfort. In addition, regulatory guidelines for cardiac monitors generally specify a maximum time to alarm of ten seconds after detection of a steep change in heart rate or a low or high heart rate. As a consequence, conventional cardiac monitors are often influenced by artifacts and suffer from a high level of false alarms, adding to the nursing burden and causing “alarm fatigue.”Deterioration of patients in general wards generally occurs slowly over several minutes or even several hours, and is often not detected until the patient has suffered harm or death.

Ballistocardiography is the measurement of the recoil movements of the body which result from motion of the heart and blood in the circulatory system. Transducers are available which are able to detect minute movements of the body produced by the acceleration of the blood as it moves in the circulatory system. For example, U.S. Pat. No. 4,657,025 to Orlando, which is incorporated herein by reference, describes a device for sensing heart and breathing rates in a single transducer. The transducer is an electromagnetic sensor constructed to enhance sensitivity in the vertical direction of vibration produced on a conventional bed by the action of the patient's heartbeat and breathing functions. The transducer is described as achieving sufficient sensitivity with no physical coupling between the patient resting in bed and the sensor placed on the bed away from the patient.

The following patents and patent application publications, all of which are incorporated herein by reference, may also be of interest:
U.S. Pat. No. 4,657,026 to Tagg;
U.S. Pat. No. 5,235,989 to Zomer;
U.S. Pat. No. 5,957,861 to Combs;
U.S. Pat. No. 6,383,142 to Gavriely;
U.S. Pat. No. 6,436,057 to Goldsmith et al.;
U.S. Pat. No. 6,856,141 to Ariav;
U.S. Pat. No. 6,984,993 to Ariav;
U.S. Pat. No. 6,134,970 to Kumakawa;
U.S. Pat. No. 5,964,720 to Pelz;
US Patent Application 2005/0119586 to Coyle et al.;
US Patent Application 2006/0084848 to Mitchnick;
U.S. Pat. No. 5,743,263 to Baker;
U.S. Pat. No. 5,540,734 to Zabara;
U.S. Pat. No. 6,375,621 to Sullivan;
US Patent Application 2003/0045806 to Brydon;
U.S. Pat. No. 6,984,207 to Sullivan;
U.S. Pat. No. 7,025,729 to de Chazal;
U.S. Pat. No. 6,980,679 to Jeung;
US Patent Application Publication 2007/0249952 to Rubin et al.; and
US Patent Application 2007/0156031 to Sullivan.

An article by Shochat M et al., entitled, “PedemaTOR: Innovative method for detecting pulmonary edema at the pre-clinical stage,” undated, available at http: //www.isramed.info /rsmm_rabinovich/pedemator.htm, which is incorporated herein by reference, describes an impedance monitor for pre-clinical detection of pulmonary edema. The impedance monitor measures “internal thoracic impedance,” which is roughly equal to lung impedance, by automatically calculating skin-electrode impedance and subtracting it from the measured transthoracic impedance.

US Patent Application Publication 2007/0177785 to Raffy, which is incorporated herein by reference, describes a method for identifying pulmonary embolisms, including tracing, by a radiologist, the pulmonary artery and pulmonary veins visible in a set of CT images and identifying the arteries and veins. The radiologist's identification of the pulmonary arteries and pulmonary veins is received by an image analyzer and combined with the analyzer's identification of the pulmonary arteries to form a combined identification. The analyzer reviews this combined identification of the pulmonary arteries to detect any pulmonary embolisms. The radiologist's identification of any pulmonary embolisms is compared with the analyzer's identification of any pulmonary embolisms to determine if there are any embolisms identified by the analyzer that were not identified by the radiologist.

The following articles, which are incorporated herein by reference, may also be of interest:
Bouillon T., et al., “Opiod-induced respiratory depression is associated with increased tidal volume variability,” European Journal of Anasthesiology, 2003; 20: 127-133.
Alihanka J., et al., “A new method for long-term monitoring of the ballistocardiogram, heart rate, and respiration,” Am J Physiol Regul Integr Comp Physiol 240:384-392 (1981).
Bentur, L. et al., “Wheeze monitoring in children for assessment of nocturnal asthma and response to therapy,” Eur Respir J 21(4):621-626 (2003).
Chang, A. B. et al., “Cough, airway inflammation, and mild asthma exacerbation,” Archives of Disease in Childhood 86:270-275 (2002).
Hsu, J. Y., et al., “Coughing frequency in patients with persistent cough: assessment using a 24 hour ambulatory recorder,” Eur Respir J 7:1246-1253 (1994).
Mack, D., et al., “Non-invasive analysis of physiological signals: NAPS: A low cost, passive monitor for sleep quality and related applications,” University of Virginia Health System (undated).
Korpas J., “Analysis of the cough sound: an overview,” Pulmonary Pharmacology 9:261-268 (1996).
Thorpe, C.; Toop, L.; and Dawson, K., “Towards a quantitative description of asthmatic cough sounds,” Eur. Respir. J, 1992, 5, 685-692.
Hirtum, A.; Berckmans, D.; Demuynck, K.; and Compernolle, D., “Autoregressive Acoustical Modelling of Free Field Cough Sound,” Proc. International Conference on Acoustics, Speech and Signal Processing, volume I, pages 493-496, Orlando, U.S.A., May 2002.
Piirila, P., et al., “Objective assessment of cough,” Eur Respir J 8:1949-1956 (1995).
Salmi, T., et al., “Long-term recording and automatic analysis of cough using filtered acoustic signals and movements on static charge sensitive bed,” Chest 94:970-975 (1988).
Salmi, T., et al., “Automatic analysis of sleep records with static charge sensitive bed,” Electroencephalography and Clinical Neurophysiology 64:84-87 (1986).
Stegmaier-Stracca, P. A., et al., “Cough detection using fuzzy classification,” Symposium on Applied Computing, Proceedings of the 1995 ACM Symposium on Applied Computing, Nashville, Tenn., United States, pp. 440-444 (1995).
Van der Loos, H. F. M., et al., “Unobtrusive vital signs monitoring from a multisensor bed sheet,” RESNA '2001, Reno, Nev., Jun. 22-26, 2001.
Waris, M., et al., “A new method for automatic wheeze detection,” Technol Health Care 6(1):33-40 (1998).
Katz, M.; Gill, P.; and Newman, R., “Detection of preterm labor by ambulatory monitoring of uterine activity: a preliminary report”, Obstetrics & Gynecology 1986; 68:773-778.
“British Guideline on the Management of Asthma: A national clinical guideline,” British Thoracic Society, Scottish Intercollegiate Guidelines Network, Revised edition April 2004.
Brenner, B. E., et al., “The clinical presentation of acute asthma in adults and children,” In Brenner, B E, ed. Emergency Asthma (New York: Marcel Dekker, 1999:201-232).
Baren, et al., “Current concepts in the ED treatment of pediatric asthma,” Respiratory Medicine Consensus Reports (Thomson American Health Consultants, Dec. 28, 2003).
“Managing Asthma,” KidsHealth website, (kidshealth.org/parent/medical/lungs/asthma_mgmt.html).
“Signs and symptoms of asthma,” Indian Chest Society (Mumbai, India) (http://www.indianchestsociety.org/symptomsofasthma.htm).
“Breathing easier with asthma,” Intermountain Health Care Clinical Education Services (http://www.ihc.com/xp/ihc/documents/clinical/101/3/1/asthma_breathe.pdf).
“Medical Mutual clinical practice guidelines for asthma: 2004,” Medical Mutual (Cleveland, Ohio) (http://www.medmutual.com/provider/pdf/resources/asthma4.pdf).
“Peak flow learning center,” National Jewish Medical and Research Center (http://www.njc.org/diseaseinfo/diseases/asthma/living/tools/peak/index.aspx).
Mintzer, R., “What the teacher should know about asthma attacks,” Family Education Network (http://www.familyeducation.com/article/0,1120,65-415,00.html).
“‘Does my child have asthma?’,” Solano Asthma Coalition, American Lung Association of the East Bay (http://www.alaebay.org/misc_pdf/solano_asthma_coalition child_asthma.pdf).
Poteetm, J. “Asthma” (http://www.nku.edu/˜rad350/asthmajp.html).
Plaut, T., “Tracking and treating asthma in young children,” J Respir Dis Pediatrician 5(2):67-72 (2003).
Fitzpatrick, M. F., et al., “Snoring, asthma and sleep disturbances in Britain: A community based survey,” Eur Respiratory J 1993; 6:531-5.
Jobanputra, P., et al., “Management of acute asthma attacks in general practice,” Br J Gen Pract 1991; 41:410-3.
Lim, T. O., et al., “Morbidity associated with asthma and audit of asthma treatment in outpatient clinics,” Singapore Med J 1992; 33:174-6.
Madge, P. J., et al., “Home nebuliser use in children with asthma in two Scottish Health Board Areas,” Scott Med J 1995:40:141-3.
Watanabe, T., et al., “Noncontact Method for Sleep Stage Estimation,” IEEE Transactions on Biomedical Engineering, No 10, Vol. 51, October 2004.
Yongjoon, C., et al., “Air mattress sensor system with balancing tube for unconstrained measurement of respiration and heart beat movements”, 2005 Physiol. Meas. 26 413-422.
Rechtschaffen A., Kales A. Manual of standardized terminology, techniques and scoring system for sleep for sleep stages of human subjects. Los Angeles: UCLA brain information services/brain research institute, 1968.
Whitney, C. W., Gottlieb D J, Redline S, Norman R G, Dodge R R, Shahar E, Surovec S and Nieto F J, “Reliability of scoring respiratory disturbance indices and sleep staging,” Sleep, 1998, Nov. 2; 21(7): 749-757.
Hudgel, D. W., R. J. Martin, B. Johnson, and P. Hill, “Mechanics of the respiratory system during sleep in normal humans,” J. Appl. Physiol., vol. 5, pp. 133-137, 1984.
Kandtelhardt, J. W., T. Penzel, S. Rostig, H. F. Becker, S. Halvin, and A. Bunde, Breathing during REM and non-REM sleep: correlated versus uncorrelated behavior,” Physica. A., vol. 319, pp. 447-457, 2003.
Oppenheim, A. V., and R. W. Schafer, Discrete-Time Signal Processing, Prentice-Hall, 1989, pp. 311-312.
Li, Q. and A. Barron, “Mixture density estimation,” Advances in neural information processing systems, vol. 12, pp. 279-285, MIT press, 2000.
Dempster, A. P., N. M. Laird, and D. B. Rubin, “Maximum likelihood from incomplete data via the EM algorithm,” Journal of the royal statistical society, vol. 39 B, pp. 1-38, 1977.
Bilmes, J., “A gentle tutorial on the EM algorithm and its application to parameter estimation for Gaussian mixture and hidden Markov models,” Technical report, University of Berkely, ICSI-TR-97-021, 1997. Available at http://www.citeseer.nj.nec.com/bilmes98gentle.html.
Schwarz, G., “Estimating the dimension of a model,” Annals of statistics, vol. 6, pp. 461-464, 1978.
Sorvoja, H. and Myllylä, R., “Noninvasive blood pressure measurement methods,” Molecular and Quantum Acoustics. vol. 27, 2006.
O'Connor CJ et al, “Identification of endotracheal tube malpositions using computerized analysis of breath sounds via electronic stethoscopes,” Anesth Analg 2005; 101:735-9.

U.S. Pat. No. 7,077,810 to Lange et al., which is assigned to the assignee of the present application and is incorporated herein by reference, describes a method for predicting an onset of a clinical episode, the method including sensing breathing of a subject, determining at least one breathing pattern of the subject responsively to the sensed breathing, comparing the breathing pattern with a baseline breathing pattern, and predicting the onset of the episode at least in part responsively to the comparison.

U.S. Provisional Patent Applications 60/541,779, 60/674,382 and 60/692,105, PCT Publication WO 05/074361 to Lange et al., US Patent Application Publication 2006/0241510 to Halperin et al., and US Patent Application Publication 2007/0118054 to Pinhas et al., all of which are assigned to the assignee of the present application and incorporated herein by reference, describe various methods and systems for clinical episode prediction and monitoring.

The inclusion of the foregoing references in this Background section does not imply that they constitute prior art or analogous art with respect to the invention disclosed herein.

### SUMMARY OF THE INVENTION

Embodiments of the present invention provide methods and systems for monitoring patients for the occurrence or recurrence of a physiological event, for example, a chronic illness or ailment. This monitoring assists the patient or healthcare provider in treating the ailment or mitigating the effects of the ailment. Embodiments of the present invention provide techniques for monitoring vital and non-vital signs using automated sensors and electronic signal processing, in order to detect and characterize the onset of a physiological event, and, for some applications, to treat the event, such as with therapy or medication.

Some embodiments of the present invention provide methods and systems for monitoring various medical conditions, such as chronic medical conditions. The chronic medical condition may be, for example, asthma, apnea, insomnia, congestive heart failure, and/or hypoglycemia, such as described hereinbelow. Some embodiments of the present invention provide methods and systems for monitoring an acute medical condition, such as may occur during hospitalization before or after surgery, or during hospitalization because of exacerbation of congestive heart failure.

In embodiments of the present invention, the system typically comprises a motion acquisition module, a pattern analysis module, an output module, a control unit that is configured to carry out one or more steps of the methods described herein (such as analytical steps), and a sensor that is configured to carry out one or more of the sensing steps of the methods described herein.

There is therefore provided, in accordance with an embodiment of the invention, apparatus including:

at least one sensor, configured to sense a physiological parameter of a subject and to sense large body movement of the subject;

an output unit; and

a control unit, configured to:
monitor a condition of the subject by analyzing the physiological parameter and the sensed large body movement; and
drive the output unit to generate an alert upon detecting a deterioration of the monitored condition.

In an embodiment, the control unit is configured to determine an activity level of the subject based on sensed large body movements of the subject, and to monitor the condition of the subject by analyzing the physiological parameter in combination with the activity level of the subject.

In an embodiment, the physiological parameter is a respiratory rate of the subject, and the at least one sensor is configured to sense the respiratory rate.

In an embodiment, the physiological parameter is a heart rate of the subject, and the at least one sensor is configured to sense the heart rate.

In an embodiment, the physiological parameter is a blood oxygen level of the subject, and the at least one sensor is configured to sense the blood oxygen level.

In an embodiment, the sensor includes a pulse oximeter.

In an embodiment, the at least one sensor includes a first sensor configured to sense the physiological parameter, and a second sensor configured to sense the large body movement.

In an embodiment, the at least one sensor includes a same sensor that senses both the physiological parameter and the large body movement.

In an embodiment, the at least one sensor is configured to sense the physiological parameter by deriving the physiological parameter from the large body movement.

In an embodiment, the control unit is configured to:
receive a specified range of values for the physiological parameter, and
drive the output unit to generate the alert only upon finding that the sensed physiological parameter falls outside the specified range over 50% of the times it is sensed during a period having a duration of at least 30 seconds

In an embodiment, the control unit is configured to:
receive a specified range of values for the physiological parameter,
calculate a representative value of the physiological parameter responsively to sensing the physiological parameter at least once every 10 seconds during a period having a duration of at least 30 seconds, and
drive the output unit to generate the alert only upon finding that the representative value of the physiological parameter falls outside the specified range during the period.

In an embodiment, the condition includes pressure sores of the subject, and the control unit is configured to predict an onset of the pressure sores by analyzing in combination the physiological parameter and the sensed large body movement.

In an embodiment, the control unit is configured to detect a change in posture of the subject, and to decrease a likelihood of predicting the onset of the pressure sores in response to detecting the change in posture.

In an embodiment, the control unit is configured to decrease a likelihood of predicting the onset of the pressure sores in response to determining that a sensed large body movement is associated in time with a change in a sensed aspect of the physiological parameter.

In an embodiment, the physiological parameter includes respiration of the subject.

In an embodiment, the control unit is configured to increase a likelihood of predicting the onset of the pressure sores in response to determining that a sensed large body movement is not associated in time with a change in a sensed aspect of the physiological parameter.

In an embodiment, the control unit is configured to identify the sensed large body movement and to minimize an interfering effect of the sensed large body movement on the analysis of the physiological parameter.

In an embodiment, the control unit is configured to minimize the interfering effect of the sensed large body movement by rejecting sensor data indicative of the physiological parameter acquired during at least some large body movements of the subject.

There is further provided, in accordance with an embodiment of the invention, apparatus for use with a subject, including:

a sensor assembly, configured to be placed in a vicinity of a subject site, and including:
a semi-rigid plate; and
a motion sensor coupled to the plate, the motion sensor configured to sense a motion-related parameter of the subject without contacting or viewing the subject or clothes the subject is wearing;

an output module; and

a control unit, configured to:
derive from the motion-related parameter at least one clinical parameter of the subject,
analyze the at least one clinical parameter to detect a clinical deterioration of the subject, and
drive the output module to generate an output indicative of the deterioration.

In an embodiment, the clinical parameter is selected from the group consisting of: a heartbeat-related parameter and a breathing-related parameter, and the control unit is configured to derive the selected clinical parameter from the motion-related parameter.

In an embodiment, the subject site includes at least one site selected from the group consisting of: a bed and a chair.

In an embodiment, the motion sensor includes a first motion sensor and the semi-rigid plate includes a first semi-rigid plate, and the sensor assembly further includes a second semi-rigid plate and a second motion sensor coupled to the second semi-rigid plate, and a flexible connecting element that couples the first and second plates to one another.

In an embodiment, the semi-rigid plate includes a non-plastic material.

In an embodiment, the semi-rigid plate includes cardboard.

In an embodiment, the motion sensor includes a first motion sensor, and the sensor assembly further includes a second motion sensor coupled to the semi-rigid plate, and the control unit is configured to test at least the first sensor by:

driving the first sensor to generate vibration in the plate, and

sensing the vibration using the second sensor.

There is still further provided, in accordance with an embodiment of the invention, apparatus including:

a sensor assembly, configured to be placed in contact with a bed, and including:
a semi-rigid plate; and
a motion sensor coupled to the plate, the motion sensor configured to sense a motion-related parameter of the subject without contacting or viewing the subject or clothes the subject is wearing;

an output module; and

a control unit, configured to:
detect a relocation of the subject by analyzing the motion-related parameter, the relocation selected from the group consisting of: entry of the subject into the bed, and exit of the subject from the bed; and
drive the output module to generate an output responsively to the detection.

In an embodiment, the control unit is configured to detect the entry into the bed upon detecting large body movement of the subject followed by continuous motion of the subject.

In an embodiment, the control unit is configured to detect the exit from the bed upon detecting large body movement of the subject followed by a lack of motion indicated by the motion-related parameter.

There is yet further provided, in accordance with an embodiment of the invention, apparatus for use with a subject, including:

a sensor assembly, configured to be placed in a vicinity of a subject site, and including:
two semi-rigid plates;
a flexible connecting element that couples the two semi-rigid plates to one another; and
two motion sensors coupled to the respective two plates, the motion sensors configured to sense respective motion-related parameters of the subject without contacting or viewing the subject or clothes the subject is wearing;

an output module; and

a control unit, configured to:
analyze at least one of the motion-related parameters to derive at least one clinical parameter of the subject; and
drive the output module to generate an output indicative of the clinical parameter.

There is also provided, in accordance with an embodiment of the invention, apparatus for use with an alternating pressure mattress upon which a subject lies, the apparatus including:

a sensor configured to sense respiration of the subject without contacting or viewing the subject or clothes the subject is wearing;

an output unit; and

a control unit, configured to:
identify activation of the alternating pressure mattress,
perform an analysis of the sensed respiration responsively to the identifying of the activation of the mattress, and
drive the output unit to generate an output indicative of the analysis.

There is additionally provided, in accordance with an embodiment of the invention, apparatus including:

a sensor configured to sense a physiological parameter of a subject without contacting or viewing the subject or clothes the subject is wearing;

an output unit; and

a control unit, configured to:
detect a symptom of pulmonary embolism of the subject responsively to the physiological parameter, and
drive the output unit to generate an output indicative of the symptom.

In an embodiment, the sensor is configured to sense the physiological parameter without requiring compliance by the subject or involvement by a healthcare worker caring for the subject.

There is still additionally provided, in accordance with an embodiment of the invention, apparatus including:

a sensor configured to sense a physiological parameter of a subject without contacting or viewing the subject or clothes the subject is wearing;

an output unit; and

a control unit, configured to:
identify a risk of a pulmonary embolism of the subject responsively to the physiological parameter, and
drive the output unit to generate an output indicative of the risk.

There is yet additionally provided, in accordance with an embodiment of the invention, apparatus including:

a sensor configured to sense a physiological parameter of a subject without requiring compliance by the subject or involvement by a healthcare worker caring for the subject;

a sequential compression device (SCD); and

a control unit, configured to identify an early warning sign of pulmonary embolism by analyzing the sensed physiological parameter and an aspect of operation of the SCD.

There is also provided, in accordance with an embodiment of the invention, apparatus including:

a sensor configured to sense motion of a subject without contacting or viewing the subject or clothes the subject is wearing;

an output unit; and

a control unit, configured to:
responsively to the sensed motion, identify time periods without large body movements of the subject;
monitor restlessness of the subject by analyzing a distribution of the time periods without the large body movements; and
drive the output unit to generate an output indicative of the restlessness.

There is further provided, in accordance with an embodiment of the invention, a method including:

sensing a physiological parameter of a subject in a stretcher without requiring compliance by the subject or involvement by a healthcare worker caring for the subject; and

generating an output indicative of the parameter.

In an embodiment, sensing the parameter includes sensing a respiration rate of the subject.

In an embodiment, sensing the parameter includes sensing a heart rate of the subject.

In an embodiment, sensing includes sensing the parameter without contacting or viewing the subject or clothes the subject is wearing.

There is still further provided, in accordance with an embodiment of the invention, apparatus including:

a stretcher;

a sensor, coupled to the stretcher, and configured to sense a physiological parameter of a subject in the stretcher without requiring compliance by the subject or involvement by a healthcare worker caring for the subject; and

an output unit, configured to generate an output indicative of the parameter.

In an embodiment, the parameter includes a respiration rate of the subject, and the sensor is configured to sense the respiration rate.

In an embodiment, the parameter includes a heart rate of the subject, and the sensor is configured to sense the heart rate.

In an embodiment, the sensor is configured to sense the parameter without contacting or viewing the subject or clothes the subject is wearing.

There is yet further provided, in accordance with an embodiment of the invention, apparatus including:

a plurality of sensors cascaded one to the next, configured to sense a respiration-related parameter of a subject without contacting or viewing the subject or clothes the subject is wearing; and

an output unit, configured to generate an output indicative of the parameter.

There is also provided, in accordance with an embodiment of the invention, apparatus including:

a sensor configured to sense motion of a subject without contacting or viewing the subject or clothes the subject is wearing, and generate a motion-related signal responsively to the motion;

an output unit; and

a control unit, configured to:
generate a heart rate signal by demodulating the motion-related signal at a frequency between 8 and 20 Hz; and
drive the output unit to generate an output responsively to the heart rate signal.

There is additionally provided, in accordance with an embodiment of the invention, apparatus including:

a sensor configured to sense motion of a subject, and generate a motion-related signal responsively to the motion;

an output unit; and

a control unit, configured to:
demodulate the motion-related signal using a plurality of band pass filters having respective frequency ranges, to generate a demodulated signal,
select one of filters that generates the best demodulated signal,
generate a heart rate signal by demodulating the motion-related signal using the selected one of the filters, and
drive the output unit to generate an output indicative of the heart rate signal.

In an embodiment, the control unit is configured to demodulate, select the one of the filters, generate the heart rate signal, and drive the output unit a plurality of times.

There is still additionally provided, in accordance with an embodiment of the invention, apparatus including:

at least one sensor, configured to sense respiration and coughing of the subject;

an output unit; and

a control unit, configured to:
receive a baseline respiration rate of the subject expressible as a number of breaths per minute;
responsively to the sensed respiration, monitor an ongoing respiration rate of the subject expressible as a number of breaths per minute,
responsively to the sensed coughing, monitor an ongoing rate of coughing events of the subject expressible as a number of coughing events per hour,
assign a score responsively at least in part to the respiration rate and the rate of coughing events, wherein a change in the score based on an increase of b percent in breaths per minute of the ongoing respiration rate versus the baseline respiration rate is the same as a change in the score based on an increase in the rate of coughing events for some rate of coughing events that is between 0.1 and 2.0 times b coughs per hour, and
drive the output unit to generate an output indicative of the score.

In an embodiment, the control unit is configured to receive the baseline respiration rate by analyzing the sensed respiration during a baseline measurement period prior to the monitoring of the ongoing respiration rate.

In an embodiment, the at least one sensor includes a first sensor configured to sense the respiration, and a second sensor configured to sense the coughing

There is yet additionally provided, in accordance with an embodiment of the invention, apparatus including:

at least one sensor, configured to sense respiration and coughing of the subject;

an output unit; and

a control unit, configured to:
responsively to the sensed respiration, find a respiration rate of the subject,
responsively to the sensed coughing, find a rate of coughing events of the subject,
assign a score responsively at least in part to the respiration rate and the rate of coughing events, the score varying close to linearly with respect to the monitored respiration rate and with respect to the rate of coughing events, and
drive the output unit to generate an output indicative of the score.

There is also provided, in accordance with an embodiment of the invention, apparatus including:

a sensor assembly, which includes:
a motion sensor, configured to be placed in or under a reclining surface; and
a sensor wireless communication module, coupled to the motion sensor;

a control unit wireless communication module, configured to wirelessly communicate with the sensor wireless communication module; and

a control unit, which is coupled to the control unit wireless communication module, and which is configured to:

receive, via the sensor and control unit wireless communication modules, an input to the motion sensor provided by a healthcare provider via the reclining surface, and

register the sensor unit responsively to the input.

There is further provided, in accordance with an embodiment of the invention, apparatus including:

a first sensor assembly, which includes:
a first motion sensor, configured to be placed in or under a reclining surface, and to sense motion of a subject on the reclining surface, and generate a motion signal responsively to the motion; and
a sensor wireless communication module, coupled to the motion sensor;

a second sensor, configured to sense a parameter of the subject, and to generate a parameter signal responsively to the parameter;

a control unit wireless communication module, configured to wirelessly communicate with the sensor wireless communication module; and

a control unit, which is coupled to the control unit wireless communication module, and which is configured to:
receive, via the sensor and control unit wireless communication modules, the motion signal, and
register the sensor unit responsively to detecting a correlation between the motion signal and the parameter signal.

In an embodiment, the apparatus includes a wire, which couples the second sensor to the control unit.

In an embodiment, the second sensor includes a second motion sensor.

In an embodiment, the second sensor includes a physiological sensor configured to come in contact with the subject.

There is still further provided, in accordance with an embodiment of the invention, a method including:

identifying that a subject suffers from sleep apnea;

applying positive airway pressure (PAP) to the subject via a mask placed on a face of the subject;

sensing a respiratory-related parameter of the subject while the mask is on the face of the subject;

assessing a need of the subject for respiratory support responsively to the respiratory-related parameter; and

in accordance with the assessed need, configuring the mask to regulate the PAP provided to the face.

In an embodiment, the control unit is configured to regulate the PAP by regulating a distance of the mask from the face of the subject.

There is yet further provided, in accordance with an embodiment of the invention, apparatus including:

a source of positive airway pressure (PAP);

a mask, coupled to the PAP source, and configured to be placed on a face of a subject;

a sensor configured to sense a respiratory-related parameter of the subject;

a control unit, configured to:
assess a need of the subject for respiratory support responsively to the respiratory-related parameter, and
in accordance with the assessed need, configure the mask to regulate the PAP provided to the face.

In an embodiment, the control unit is configured to regulate the PAP by regulating a distance of the mask from the face of the subject.

There is also provided, in accordance with an embodiment of the invention, apparatus including:

a sensor configured to sense a respiratory parameter of a subject;

an output unit; and

a control unit, configured to:
detect a symptom of alcohol withdrawal responsively to the parameter, and
drive the output unit to generate an output responsively to detecting the symptom.

There is additionally provided, in accordance with an embodiment of the invention, apparatus including:

a sensor configured to sense a physiological parameter of a subject without requiring compliance by the subject or involvement by a healthcare worker caring for the subject;

an output unit; and

a control unit, configured to:
estimate a hypnogram responsively to the parameter, and
drive the output unit to generate an output responsively to the hypnogram.

There is still additionally provided, in accordance with an embodiment of the invention, method including:

sensing a respiratory parameter of a subject while the subject sleeps;

identifying a change in pulmonary hypertension of the subject responsively to the parameter; and

generating an output indicative of the change.

There is yet additionally provided, in accordance with an embodiment of the invention, apparatus including:

a sensor configured to sense a respiratory parameter of a subject while the subject sleeps;

an output unit; and

a control unit, configured to:
identify a change in pulmonary hypertension of the subject responsively to the parameter, and
drive the output unit to generate an output indicative of the change.

There is also provided, in accordance with an embodiment of the invention, apparatus including:

a sensor configured to sense a respiratory parameter of a subject without contacting or viewing the subject or clothes the subject is wearing;

an output unit; and

a control unit, configured to:
perform an assessment of insomnia of the subject responsively to the parameter, and
drive the output unit to generate an output indicative of the assessment.

There is further provided, in accordance with an embodiment of the invention, apparatus including:

a first sensor configured to sense at least one parameter of a subject without contacting or viewing the subject or clothes the subject is wearing, the at least one parameter selected from the group consisting of: a cardiac-related parameter and a respiration-related parameter;

a second sensor configured to sense a level of blood oxygen of the subject;

an output unit; and

a control unit, configured to:
assess an accuracy of the sensed blood oxygen level responsively to the sensed parameter, and
drive the output unit to generate an output responsively to the assessed accuracy.

In an embodiment, the second sensor includes a pulse oximeter.

There is still further provided, in accordance with an embodiment of the invention, apparatus including:

a first sensor configured to sense at least one parameter of a subject without contacting or viewing the subject or clothes the subject is wearing, the at least one parameter selected from the group consisting of: a cardiac-related parameter and a respiration-related parameter;

a second sensor configured to sense a level of blood oxygen of the subject;

an output unit; and

a control unit, configured to:
detect imminent distress of the subject responsively to the sensed blood oxygen level and the sensed parameter, and
drive the output unit to generate an output indicative of the imminent distress.

In an embodiment, the control unit is configured to detect imminent respiratory depression of the subject responsively to the sensed blood oxygen level and the sensed parameter, and to generate the output indicative of the imminent respiratory depression.

There is yet further provided, in accordance with an embodiment of the invention, apparatus including:

a first sensor configured to sense at least one parameter of a subject without contacting or viewing the subject or clothes the subject is wearing, the at least one parameter selected from the group consisting of: a cardiac-related parameter and a respiration-related parameter;

a second sensor configured to be placed in contact with an external surface of an extremity of the subject, and to sense an extremity pulse of the subject;

an output unit; and

a control unit, configured to:
perform an analysis of the sensed extremity pulse in combination with the sensed parameter, and
drive the output unit to generate an output indicative of the analysis.

In an embodiment, the control unit is configured to perform the analysis by identifying an indication of pulse propagation time responsively to the sensed extremity pulse in combination with the sensed parameter.

In an embodiment, the second sensor includes a pulse oximeter.

In an embodiment, the control unit is configured to detect imminent distress of the subject responsively to the analysis, and to drive the output unit to generate the output indicative of the imminent distress.

There is also provided, in accordance with an embodiment of the invention, apparatus for use during endotracheal intubation of a subject, the apparatus including:

at least two sensors configured to sense motion of the subject, and generate respective signals responsively thereto;

an output unit; and

a control unit, configured to:
detect an adverse aspect of the intubation by analyzing respective components of the signals having a frequency of less than 20 Hz, and
drive the output unit to generate an output indicative of the adverse aspect.

In an embodiment, the sensors are configured to be coupled to an external surface of a body of the subject.

In an embodiment, first and second ones of the sensors are configured to be coupled to the external surface in respective vicinities of a left lung and a right lung of the subject, and to generate respective first and second signals responsively to the respective motion in the vicinities of the left and right lungs.

In an embodiment, the control unit is configured to detect the adverse aspect upon finding that the first and second signals have different strengths.

In an embodiment, the adverse aspect of the intubation includes malpositioning of a tube used for the intubation.

In an embodiment, the control unit is configured to analyze the respective components of the signals during performance of the intubation.

In an embodiment, the output is audible, and the output unit is configured to generate the audible output.

In an embodiment, the control unit is configured to identify a difference in ventilation effectiveness of two lungs of the subject.

In an embodiment, the adverse aspect is insertion of a tube used for the intubation into an esophagus of the subject.

There is additionally provided, in accordance with an embodiment of the invention, a method including:

performing endotracheal intubation on a subject;

sensing motion of the subject, and generating a signal responsively thereto;

detecting an adverse aspect of the intubation by analyzing a component of the signal having a frequency of less than 20 Hz; and

generating an output indicative of the adverse aspect.

In an embodiment, sensing includes coupling a sensor to an external surface of a body of the subject, and sensing the motion using the sensor.

In an embodiment, sensing includes coupling at least two sensors to the external surface, and sensing the motion using the at least two sensors.

In an embodiment, coupling includes coupling first and second ones of the sensors to the external surface in respective vicinities of a left lung and a right lung of the subject, and sensing includes generating respective first and second signals with the first and second sensors responsively to the respective motion in the vicinities of the left and right lungs.

In an embodiment, detecting the adverse aspect includes detecting the adverse aspect upon finding that the first and second signals have different strengths.

In an embodiment, the adverse aspect of the intubation includes malpositioning of a tube used for the intubation.

In an embodiment, sensing includes sensing the motion while performing the intubation.

In an embodiment, generating the output includes generating an audible output.

In an embodiment, sensing the parameter includes using a plurality of sensors to identify a difference in ventilation effectiveness of two lungs of the subject.

In an embodiment, the adverse aspect is insertion of a tube used for the intubation into an esophagus of the subject.

There is still additionally provided, in accordance with an embodiment of the invention, a method including:

coupling a sensor to an external surface of a body of a subject who has undergone a tracheotomy;

sensing, with the sensor, an adverse aspect of the tracheotomy; and

generating an output indicative of the adverse aspect.

In an embodiment, the adverse aspect of the tracheotomy includes malpositioning of a tube inserted during the tracheotomy.

There is yet additionally provided, in accordance with an embodiment of the invention, a method including:

performing a tracheotomy on a subject;

sensing, using a mechanical sensor, a parameter of the subject;

identifying an adverse aspect of the tracheotomy responsively to the parameter; and

generating an output indicative of the adverse aspect.

In an embodiment, sensing includes sensing while performing the tracheotomy.

There is also provided, in accordance with an embodiment of the invention, a method including:

identifying a patient as one who is undergoing chemotherapy;

sensing respiration of the patient without contacting or viewing the subject or clothes the subject is wearing;

analyzing the sensed respiration to identify an onset of a condition selected from the group consisting of: chronic heart failure and pulmonary edema; and

generating an output indicative of the onset.

There is further provided, in accordance with an embodiment of the invention, method including:

identifying a subject as suffering from renal failure;

sensing respiration of the subject without contacting or viewing the subject or clothes the subject is wearing;

analyzing the sensed respiration;

identifying a need for intervention with respect to the renal failure in response to analyzing the sensed respiration; and

generating an output responsively to the identifying.

There is still further provided, in accordance with an embodiment of the invention, apparatus including:

a sensor configured to sense motion of a subject without contacting or viewing the subject or clothes the subject is wearing;

an output unit; and

a control unit, configured to:
receive a specified range of values for a clinical parameter,
responsively to the sensed motion, calculate a value of the clinical parameter of the subject at least once every 10 seconds, during a period having a duration of at least 30 seconds, and
only upon finding that the value falls outside the specified range over 50% of the times it is calculated throughout the period, drive the output unit to generate an alert.

In an embodiment, the duration is at least 60 seconds, and the control unit is configured to calculate the representative value of the clinical parameter during the period having the duration of at least 60 seconds.

In an embodiment, the clinical parameter is heart rate.

In an embodiment, the clinical parameter is respiration rate.

There is yet further provided, in accordance with an embodiment of the invention, apparatus including:

a sensor configured to sense motion of a subject without contacting or viewing the subject or clothes the subject is wearing;

an output unit; and

a control unit, configured to:
receive a specified range of values for a clinical parameter,
responsively to the sensed motion, calculate respective raw values of the clinical parameter of the subject at least once every 10 seconds, during a period having a duration of at least 30 seconds,
calculate a representative value based on the raw values, and
only upon finding that the representative value falls outside the specified range, drive the output unit to generate an alert.

In an embodiment, the duration is at least 60 seconds, and the control unit is configured to calculate the raw values of the clinical parameter during the period having the duration of at least 60 seconds.

In an embodiment, the clinical parameter is heart rate.

In an embodiment, the clinical parameter is respiration rate.

There is also provided, in accordance with an embodiment of the invention, apparatus including:

a sensor configured to sense motion of a subject without contacting or viewing the subject or clothes the subject is wearing;

an output unit; and

a control unit, configured to:
receive an indication of a baseline value for a clinical parameter, responsively to the sensed motion, calculate a value of the clinical parameter of the subject at least times, during a period having a duration of at least 10 seconds, and
only upon finding that the value is at least a threshold percentage different from the baseline value over 50% of the times it is calculated throughout the period, drive the output unit to generate an alert.

In an embodiment, the duration is at least 30 seconds or at least 60 seconds.

In an embodiment, the duration is at least one hour, and the control unit is configured to calculate the value of the clinical parameter during the period having the duration of at least one hour.

There is additionally provided, in accordance with an embodiment of the invention, apparatus including:

a sensor configured to sense motion of a subject without contacting or viewing the subject or clothes the subject is wearing;

an output unit; and

a control unit, configured to:
receive an indication of a baseline value for a clinical parameter,
responsively to the sensed motion, calculate respective raw values of the clinical parameter of the subject at least times, during a period having a duration of at least 10 seconds,
calculate a representative value based on the raw values, and
only upon finding that the representative value is at least a threshold percentage different from the baseline value, drive the output unit to generate an alert.

In an embodiment, the duration is at least 30 seconds or at least 60 seconds.

In an embodiment, the duration is at least one hour, and the control unit is configured to calculate the raw values of the clinical parameter during the period having the duration of at least one hour.

There is still additionally provided, in accordance with an embodiment of the invention, apparatus including:

a sensor configured to sense an aspect of a subject, and to generate a signal responsively thereto;

an output unit; and

a control unit, configured to:
responsively to the signal, calculate a representative value of a clinical parameter of the subject, and a confidence level parameter indicative of a level of confidence for the representative value,
analyze a level of deterioration of a condition of the subject responsively to the representative value and the confidence level parameter, and
upon finding that the level of deterioration is greater than a threshold level, drive the output unit to generate an alert.

In an embodiment, the control unit is configured to calculate the confidence level parameter in real time responsively to the signal.

In an embodiment, the control unit is configured to calculate the confidence level parameter by calculating a signal-to-noise ratio in the signal.

There is yet additionally provided, in accordance with an embodiment of the invention, apparatus including:

a sensor assembly;

at least two mechanical sensors coupled to the sensor assembly such that the sensors are oriented at different angles with respect to a body of a subject when the sensor assembly is placed in a vicinity of the body, and the sensors are configured to generate respective sensor signals without contacting or viewing the subject or clothes the subject is wearing; and

a control unit, configured to receive the sensor signals, and generate an output responsively to an analysis that combines the sensor signals.

In an embodiment, the control unit is configured to perform the analysis on components of the sensor signals having a frequency of less than 20 Hz.

There is also provided, in accordance with an embodiment of the invention, an apparatus for monitoring a subject, including:

a sensor assembly;

a plurality of sensors coupled to the sensor assembly, and configured to generate respective sensor signals, whereby each sensor mechanically senses motion of the subject without contacting or viewing the subject or clothes the subject is wearing, and detects different respective noise patterns from respective sources of noise;

an output unit; and

a control unit, configured to:
generate a corrected signal by analyzing differences between the sensor signals to remove the noise generated by the sources,
assess a clinical state of the subject responsively to the corrected signal, and
drive the output unit to generate an output indicative of the clinical state.

In an embodiment, the control unit is configured to assess the clinical state by analyzing a component of the corrected signal having a frequency of less than 20 Hz.

There is further provided, in accordance with an embodiment of the invention, apparatus including:

a sensor, configured to detect movement of a subject, and to generate a movement signal;

an output module; and

a control unit, configured to:
predict an onset of pressure sores by analyzing the movement signal, and
drive the output module to generate an output indicative of the onset.

In an embodiment, the control unit is configured to detect a change in a posture of the subject, responsively to the movement signal, and to predict the onset of the sores responsively to the change in the posture.

In an embodiment, the control unit is configured to detect the change in posture by measuring a cardio-ballistic effect by analyzing the movement signal.

In an embodiment, the sensor is configured to detect the movement without contacting or viewing the subject or clothes the subject is wearing.

There is still further provided, in accordance with an embodiment of the invention, a method including:

electronically sensing movement of a subject;

calculating a level of risk of pressure sore development responsively to a level of the movement.

In an embodiment, sensing includes generally continuously sensing the movement.

In an embodiment, calculating includes calculating responsively to the level of movement measured over a period having a duration of at least 30 minutes.

There is yet further provided, in accordance with an embodiment of the invention, apparatus including:

a sensor configured to sense motion of a subject, and generate a signal responsively thereto;

an output unit; and

a control unit, configured to:
detect a level of motion of the subject responsively to the signal,
responsively to the level of motion, calculate a score indicative of a risk of the subject developing a pressure sore, and
drive the output unit to generate an output indicative of the score.

In an embodiment, the control unit is configured to detect a number of posture changes by the subject during a period of time by analyzing the signal, and to calculate the score responsively to the level of motion and the number of posture changes.

There is also provided, in accordance with an embodiment of the invention, a method including:

sensing motion of a subject, and generating a signal responsively thereto;

detecting a level of motion of the subject responsively to the signal;

responsively to the level of motion, calculating a score indicative of a risk of the subject developing a pressure sore; and

generating an output indicative of the score.

In an embodiment, the method includes detecting a number of posture changes by the subject during a period of time by analyzing the signal, and calculating the score includes calculating the score responsively to the level of motion and the number of posture changes.

In an embodiment, the method includes evaluating a level of compliance with a protocol responsively to the score.

There is additionally provided, in accordance with an embodiment of the invention, apparatus for use with a bed, the apparatus including:

a sensor coupled to the bed, and configured to sense motion of a subject in the bed, and generate a motion signal; and

a control unit, configured to:
detect a plurality of postures of the subject by analyzing the motion signal at a respective plurality of points in time, and
automatically log the detected postures.

There is still additionally provided, in accordance with an embodiment of the invention, apparatus including:

a sensor configured to sense an aspect of a subject, and generate a signal responsively thereto;

an output unit; and

a control unit, configured to:
receive, for each of a plurality of wake states, respective specified ranges of values for a clinical parameter,
determine that the subject is in one of the wake states,
responsively to the signal, calculate a representative value of the clinical parameter of the subject, and
drive the output unit to generate an alert if the representative value falls outside the one of the specified ranges corresponding to the one of the wake states of the subject.

In an embodiment, the wake states include a sleep state and an awake state.

In an embodiment, the wake states include an REM sleep state, a non-REM sleep state, and an awake state.

In an embodiment, the clinical parameter is heart rate or respiration rate.

There is yet additionally provided, in accordance with an embodiment of the invention, apparatus including:

a sensor configured to sense a physiological parameter of a subject without contacting or viewing the subject or clothes the subject is wearing;

an output unit; and

a control unit, configured to:
detect an onset of sepsis responsively to the parameter, and
drive the output unit to generate an output indicative of the onset.

There is also provided, in accordance with an embodiment of the invention, apparatus including:

a sensor configured to sense a physiological parameter of a subject without contacting or viewing the subject or clothes the subject is wearing;

an output unit; and

a control unit, configured to:
calculate a sepsis risk score responsively to the parameter, and
drive the output unit to generate an output indicative of the risk score.

There is further provided, in accordance with an embodiment of the invention, a method including:

testing a sensor coupled to a semi-rigid plate by driving the sensor to generate vibration in the plate, and sensing the vibration, and the sensor is configured to sense a physiological parameter of a subject without contacting or viewing the subject or clothes the subject is wearing;

after testing the sensor, using the sensor to sense the physiological parameter; and

generating an output responsively to the parameter.

There is still further provided, in accordance with an embodiment of the invention, apparatus including:

a sensor assembly including:
a semi-rigid plate; and
first and second sensors coupled to the semi-rigid plate, which sensors are configured to sense a physiological parameter of a subject without contacting or viewing the subject or clothes the subject is wearing;

an output unit; and

a control unit, configured to:
test the first sensor by driving the first sensor to generate vibration in the plate, and sense the vibration using the second sensor,
after testing the first sensor, sense the physiological parameter using the first sensor, and
drive the output unit to generate an output responsively to the parameter.

There is yet further provided, in accordance with an embodiment of the invention, apparatus including:

a sensor configured to sense an aspect of a subject without contacting or viewing the subject or clothes the subject is wearing, and generate a signal responsively thereto;

an output unit; and

a control unit, configured to:
determine a level of large body movement of the subject,
calculate a representative value of a clinical parameter of the subject responsively to the signal and the level of large body movement, and
drive the output unit to generate an output indicative of the representative value.

In an embodiment, the aspect of the subject includes motion of the subject, the sensor is configured to generate the signal responsively to the motion, and the control unit is configured to determine the level of large body movement responsively to the signal.

In an embodiment, the level of large body movement includes an activity level of the subject, and the control unit is configured to determine the activity level based on the large body movement, and to calculate the representative value of the clinical parameter responsively to the signal and the activity level.

In an embodiment, the control unit is configured to determine the activity level by identifying whether the subject is in an active mode or in a rest mode.

There is also provided, in accordance with an embodiment of the invention, apparatus including:

a sensor configured to sense an aspect of a subject without contacting or viewing the subject or clothes the subject is wearing, and generate a signal responsively thereto;

an output unit; and

a control unit, configured to:
responsively to the signal, calculate a plurality of representative values of a clinical parameter of the subject at a plurality of respective times,
identify a trend over time in the representative values,
calculate a level of deterioration of a condition of the subject responsively to at least one of the representative values and the trend, and
drive the output unit to generate an alarm if the level of deterioration crosses a threshold value.

There is additionally provided, in accordance with an embodiment of the invention, apparatus including:

a first sensor configured to sense motion of a subject without contacting or viewing the subject or clothes the subject is wearing, and generate a motion signal responsively thereto;

a second sensor configured to be placed in contact with an external surface of an extremity of the subject, sense an extremity pulse of the subject, and generate an extremity pulse signal responsively thereto;

an output unit; and

a control unit, configured to:
derive a central pulse signal from the motion signal,
identify a change in blood pressure of the subject by analyzing a change in a delay from detection of a pulse in the central pulse signal to detection of a pulse in the extremity pulse signal, and
drive the output unit to generate an output indicative of the change in the blood pressure.

There is still additionally provided, in accordance with an embodiment of the invention, apparatus including:

a sensor configured to sense a physiological parameter of a subject without contacting or viewing the subject or clothes the subject is wearing;

an output unit; and

a control unit, configured to:
responsively to the parameter, identify a sudden drop in systolic blood pressure of the subject, and
upon identifying the sudden drop, drive the output unit to generate an alert.

There is yet additionally provided, in accordance with an embodiment of the invention, apparatus including:

at least two sensors, configured to sense motion of a subject without contacting or viewing the subject or clothes the subject is wearing, and to sense one or more local pulses of the subject;

a plurality of second sensors configured to

an output unit;

a control unit, configured to:
determine a level of large body movement of the body of the subject responsively to the sensed motion,
calculate a pulse transit time responsively to the one or more local pulses and the level of large body movement, and
drive the output unit to generate an output indicative of the pulse transit time.

In an embodiment, the level of large body movement includes a level of activity of the subject, and the control unit is configured to determine the level of activity of the subject based on the large body movement, and to calculate the pulse transit time responsively to the one or more local pulses and the level of activity.

In an embodiment, the control unit is configured to discard the local pulses that are sensed during periods having a level of large body movement greater than a threshold level.

In an embodiment, the at least two sensors include exactly two sensors, a first one of which is configured to sense the motion without contacting or viewing the subject or the clothes the subject is wearing and to sense a first one of the local pulses without contacting or viewing the subject or the clothes the subject is wearing, and second one of which is configured to sense a second one of the local pulses.

In an embodiment, the at least two sensors include:

exactly one first sensor, which is configured to sense the motion without contacting or viewing the subject or the clothes the subject is wearing; and

two or more second sensors, configured to sense the local pulses.

There is also provided, in accordance with an embodiment of the invention, apparatus including:

a sensor configured to sense motion of a subject without contacting or viewing the subject or clothes the subject is wearing;

an output unit; and

a control unit, configured to:
measure a plurality of heart rates of the subject at a plurality of respective times by analyzing the sensed motion,
identify a risk of arrhythmia upon detecting a high level of variability in the sensed heart rates, and
drive the output unit to generate an output indicative of the risk.

In an embodiment, the control unit is configured to detect body movement of the subject from the sensed motion, and filter out a portion of the heart rates measured at a respective portion of the times during which the body movement is detected.

There is further provided, in accordance with an embodiment of the invention, apparatus including:

a sensor configured to sense an aspect of a subject without contacting or viewing the subject or clothes the subject is wearing, and generate a signal responsively thereto;

an output unit; and

a control unit, configured to:

during a baseline period, calculate, responsively to the signal, a plurality of values of a clinical parameter of the subject, and a standard deviation and mean of the values,

during a monitoring period, calculate, responsively to the signal, a representative value of the clinical parameter, and

upon finding that the representative value is greater than a factor times the standard deviation from the mean, drive the output unit to generate an alert.

The present invention will be more fully understood from the following detailed description of embodiments thereof, taken together with the drawings, in which:

### BRIEF DESCRIPTION OF THE DRAWINGS

FIG. 1 is a schematic illustration of a system for monitoring a chronic medical condition of a subject, in accordance with an embodiment of the present invention;

FIG. 2 is a schematic block diagram illustrating components of a control unit of the system of FIG. 1, in accordance with an embodiment of the present invention;

FIG. 3 is a schematic block diagram illustrating a breathing pattern analysis module of the control unit of FIG. 2, in accordance with an embodiment of the present invention;

FIGS. 4A-C are graphs illustrating the analysis of motion signals, measured in accordance with an embodiment of the present invention;

FIGS. 5A-B are schematic illustrations of a positive airway pressure (PAP) device, in accordance with an embodiment of the present invention;

FIGS. 6A-B are schematic illustrations of another PAP device, in accordance with an embodiment of the present invention;

FIG. 7 is a schematic illustration of the system of FIG. 1 applied to an intubated subject, in accordance with an embodiment of the present invention;

FIG. 8 is a flowchart schematically illustrating a method for performing respiration complexity classification and sleep stage classification, in accordance with an embodiment of the present invention;

FIG. 9 is a flowchart that schematically illustrates a method for determining whether subject movement has occurred, in accordance with an embodiment of the present invention;

FIG. 10 is a schematic illustration of an exemplary respiration signal and the maxima and minima points used for feature extraction, in accordance with an embodiment of the present invention;

FIG. 11 is a flowchart schematically illustrating a method for classifying sleep stages, in accordance with an embodiment of the present invention;

FIG. 12 includes graphs showing experimental results obtained in accordance with an embodiment of the present invention;

FIG. 13 is a schematic illustration of a sensor assembly, in accordance with an embodiment of the present invention; and

FIG. 14 is a schematic illustration of an alternative configuration of the sensor assembly of FIG. 13, in accordance with an embodiment of the present invention.

### DETAILED DESCRIPTION OF EMBODIMENTS

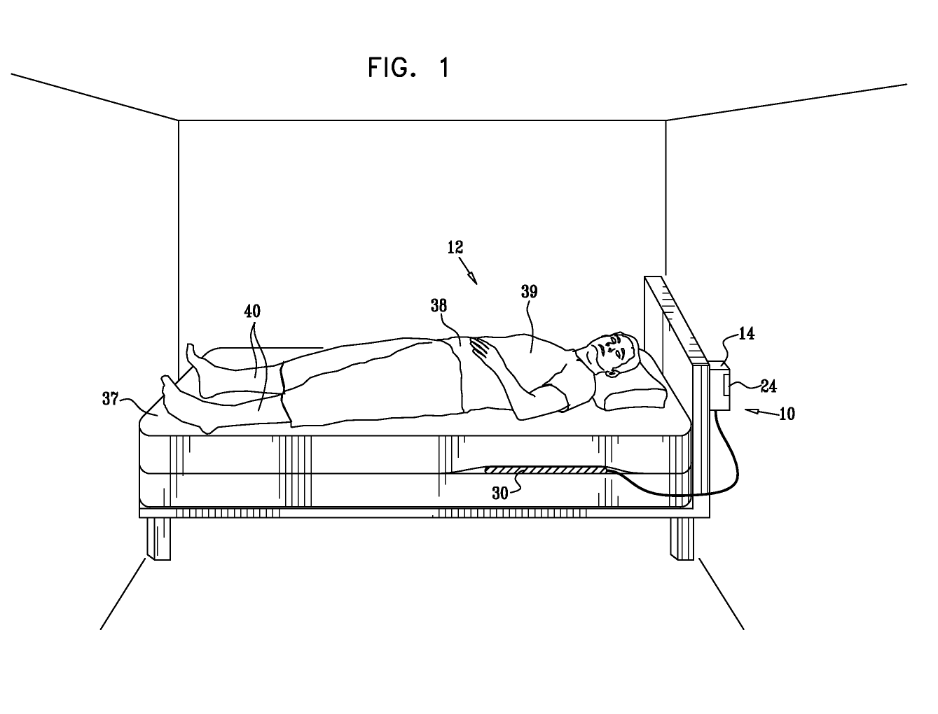

FIG. 1 is a schematic illustration of a system 10 for monitoring a chronic medical condition of a subject 12 in accordance with an embodiment of the present invention. System 10 typically comprises a motion sensor 30, a control unit 14, and a user interface (U/I) 24. For some applications, user interface 24 is integrated into control unit 14, as shown in the figure, while for other applications, the user interface and the control unit are separate units. For some applications, motion sensor 30 is integrated into control unit 14, in which case user interface 24 is either also integrated into control unit 14 or remote from control unit 14.

In some embodiments of the present invention, motion sensor 30 is a “non-contact sensor,” that is, a sensor that does not contact the body of subject 12 or clothes subject 12 is wearing. In other embodiments, motion sensor 30 does contact the body of subject 12 or clothes subject 12 is wearing. In the former embodiments, because motion sensor 30 does not come in contact with subject 12, motion sensor 30 detects motion of subject 12 without discomforting subject 12. For some applications, motion sensor 30 performs sensing without the knowledge of subject 12, and even, for some applications, without the consent of subject 12.

Motion sensor 30 may comprise a ceramic piezoelectric sensor, vibration sensor, pressure sensor, or strain sensor, for example, a strain gauge, configured to be installed under a reclining surface 37, and to sense motion of subject 12. The motion of subject 12 sensed by sensor 30, during sleep, for example, may include regular breathing movement, heartbeat-related movement, and other, unrelated body movements, as discussed below, or combinations thereof. For some applications, sensor 30 comprises a standard communication interface (e.g. USB), which enables connection to standard monitoring equipment.

All experimental results presented in the present application were measured using one or more piezoelectric sensors. Nevertheless, the scope of the present invention includes performing measurements with other motion sensors 30, such as other pressure gauges or accelerometers.

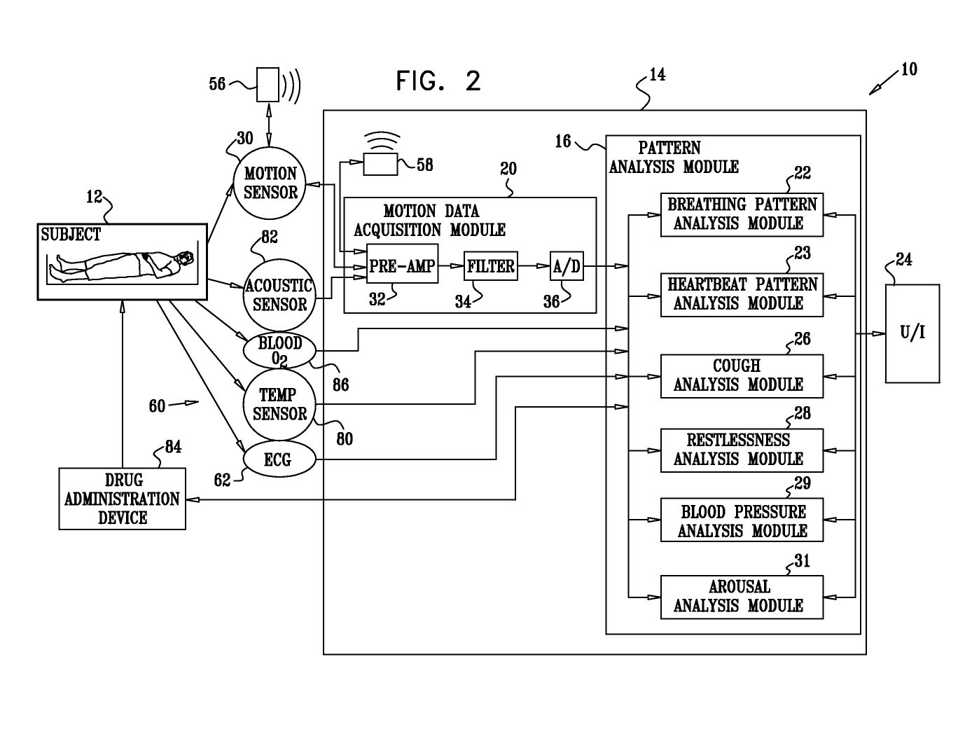

FIG. 2 is a schematic block diagram illustrating components of control unit 14 in accordance with an embodiment of the present invention. Control unit 14 typically comprises a motion data acquisition module 20 and a pattern analysis module 16. Pattern analysis module 16 typically comprises one or more of the following modules: a breathing pattern analysis module 22, a heartbeat pattern analysis module 23, a cough analysis module 26, a restlessness analysis module 28, a blood pressure analysis module 29, and an arousal analysis module 31. For some applications, two or more of analysis modules 20, 22, 23, 26, 28, 29, and 31 are packaged in a single housing. For other applications, the modules are packaged separately (for example, so as to enable remote analysis by one or more of the pattern analysis modules of breathing signals acquired locally by data acquisition module 20).

User interface 24 typically comprises a dedicated display unit, such as an LCD or CRT monitor. Alternatively or additionally, the user interface 24 comprises a wireless or wired communication port for relaying the acquired raw data and/or processed data to a remote site for further analysis, interpretation, expert review, and/or clinical follow-up. For example, the data may be transferred over a telephone line, and/or over the Internet or another wide-area network, either wirelessly or via wires.

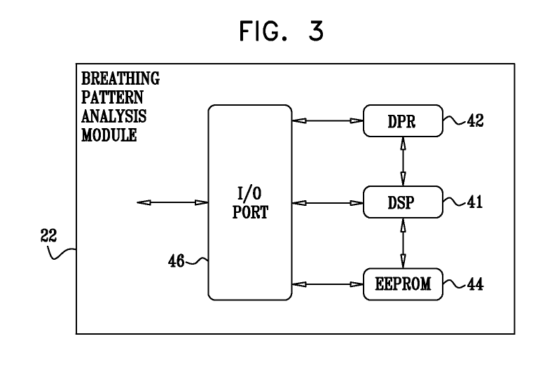

Breathing pattern analysis module 22 is configured to extract breathing patterns from the motion data, as described hereinbelow with reference to FIG. 3, and heartbeat pattern analysis module 23 is configured to extract heartbeat patterns from the motion data. Alternatively or additionally, system 10 comprises another type of sensor, such as an acoustic or air-flow sensor attached or directed at the subject's face, neck, chest, and/or back, or placed under the mattress.

In an embodiment of the present invention, system 10 comprises a temperature sensor 80 for measurement of body temperature. For some applications, temperature sensor 80 comprises an integrated infrared sensor for measurement of body temperature. Body temperature is a vital sign indicative of general status of systemic infection and inflammation. Global rise in body temperature is used as a first screening tool in medical diagnostics.

FIG. 3 is a schematic block diagram illustrating components of breathing pattern analysis module 22 in accordance with an embodiment of the present invention. Breathing pattern analysis module 22 analyzes changes in breathing patterns, typically during sleep. Breathing pattern analysis module 22 typically comprises a digital signal processor (DSP) 41, a dual port RAM (DPR) 42, an EEPROM 44, and an I/O port 46. Modules 23, 26, 28, 29, and 31 may be similar to module 22 shown in FIG. 3. For example, modules 23, 26, 28, 29, and 31 may include a digital signal processor, a dual port RAM, an EEPROM, and an I/O port similar to digital signal processor 41, dual port RAM 42, EEPROM 44, and I/O port 46.

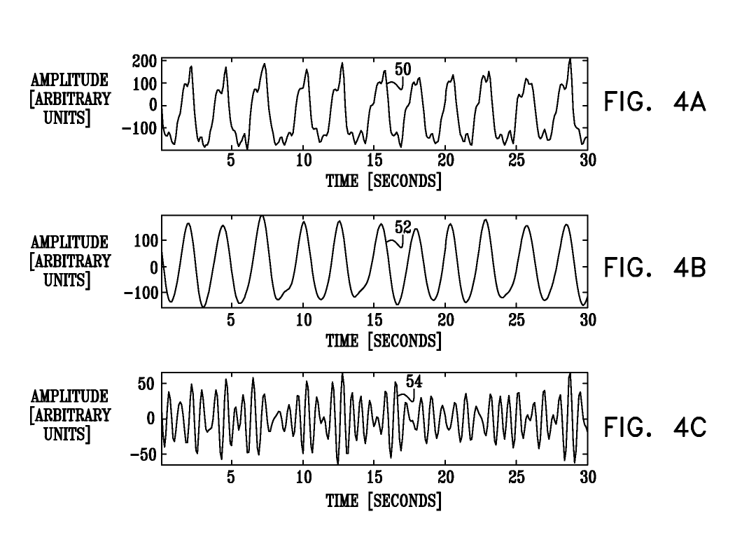

Reference is made to FIGS. 4A, 4B, and 4C, which are graphs illustrating the analysis of motion signals measured in accordance with an embodiment of the present invention. FIG. 4A shows a raw mechanical signal 50 as measured by the piezoelectric sensor under a mattress, including the combined contributions of breathing- and heartbeat-related signals. Signal 50 was decomposed into a breathing-related component 52, shown in FIG. 4B, and a heartbeat-related component 54, shown in FIG. 4C, using techniques described hereinbelow.

In an embodiment of the present invention, data acquisition module 20 is configured to non-invasively monitor breathing and heartbeat patterns of subject 12. Breathing pattern analysis module 22 and heartbeat pattern analysis module 23 are configured to extract breathing patterns and heartbeat patterns respectively from the raw data generated by data acquisition module 20, and to perform processing and classification of the breathing patterns and the heartbeat patterns, respectively. Breathing pattern analysis module 22 and heartbeat pattern analysis module 23 are configured to analyze the respective patterns in order to (a) predict an approaching clinical episode, such as an asthma attack or heart condition-related lung fluid buildup, and/or (b) monitor the severity and progression of a clinical episode as it occurs. User interface 24 is configured to notify subject 12 and/or a healthcare worker of the predicted or occurring episode. Prediction of an approaching clinical episode facilitates early preventive treatment, which generally reduces the required dosage of medication, and/or lowers mortality and morbidity. When treating asthma, for example, such a reduced dosage generally reduces the side-effects associated with high dosages typically required to reverse the inflammatory condition once the episode has begun.

Normal breathing patterns in sleep are likely to be subject to slow changes over days, weeks, months and years. Some changes are periodic due to periodic environmental changes, such as a change in seasons, or to a periodic schedule such as a weekly schedule (for example outdoor play every Saturday), or biological cycles such as the menstrual cycle. Other changes are monotonically progressive, for example, changes that occur as children grow or adults age. In some embodiments of the present invention, system 10 tracks these slow changes dynamically.

In an embodiment of the present invention, system 10 is configured to monitor parameters of the subject including, but not limited to, breathing rate, heart rate, coughing counts, expiration/inspiration ratios, augmented breaths, deep inspirations, tremor, sleep cycle, and restlessness patterns. These parameters are referred to herein, including in the claims, as “clinical parameters.”

In an embodiment of the present invention, pattern analysis module 16 combines clinical parameter data generated from one or more of analysis modules 20, 22, 23, 26, 28, 29, and 31, and analyzes the data in order to predict and/or monitor a clinical event. For some applications, pattern analysis module 16 derives a score for each parameter based on the parameter's deviation from baseline values (either for the specific patient or based on population averages). Pattern analysis module 16 optionally combines the scores, such as by computing an average, maximum, standard deviation, or other function of the scores. The combined score is compared to one or more threshold values (which may or may not be predetermined) to determine whether an episode is predicted, currently occurring, or neither predicted nor occurring, and/or to monitor the severity and progression of an occurring episode. For some applications, pattern analysis module 16 learns the criteria and/or functions for combining the individual parameter scores for the specific patient or patient group based on personal or group history. For example, pattern analysis module 16 may perform such learning by analyzing parameters measured prior to previous clinical events.

For some applications, pattern analysis module 16 is configured to analyze the respective patterns, for example, the patterns of slow changes mentioned above, in order to identify a change in baseline characteristic of the clinical parameters. For example, in order to identify the slow change in average respiration rate in sleep for a child caused by growth, the system calculates a monthly average of the respiration rate during sleep. System 10 then calculates the rate of change in average respiration rate from one month to the next month, and displays this rate of change to the subject, subject's parent, or healthcare professional. Alternatively or additionally, system 10 identifies that the average respiration rate in sleep during weekends is higher than on weekdays, and thus uses a different baseline on weekends for comparing and making a decision whether a clinical episodes is present or approaching.

In an embodiment of the present invention, system 10 monitors and logs the clinical condition of a subject over an extended period of time, such as over at least two months. During this period of time, the system also monitors and logs behavioral patterns, treatment practices and external parameters that may affect the subject's condition. System 10 calculates a score for the clinical condition of the subject based on the measured clinical parameters. The system outputs this score for use by the subject or a caregiver.

Although system 10 may monitor breathing and heartbeat patterns at any time, for some conditions it is generally most effective to monitor such patterns during sleep at night. When the subject is awake, physical and mental activities unrelated to the monitored condition often affect breathing and heartbeat patterns. Such unrelated activities generally have less influence during most nighttime sleep. For some applications, system 10 monitors and records patterns throughout all or a large portion of a night. The resulting data set generally encompasses typical long-term respiratory and heartbeat patterns, and facilitates comprehensive analysis. Additionally, such a large data set enables rejection of segments contaminated with movement or other artifacts, while retaining sufficient data for a statistically significant analysis.

Reference is again made to FIG. 2. Data acquisition module 20 typically comprises circuitry for processing the raw motion signal generated by motion sensor 30, such as at least one pre-amplifier 32, at least one filter 34, and an analog-to-digital (A/D) converter 36. Filter 34 typically comprises a band-pass filter or a low-pass filter, serving as an anti-aliasing filter with a cut-off frequency of less than one half of the sampling rate. The low-passed data is typically digitized at a sampling rate of at least 10 Hz and stored in memory. For example, the anti-aliasing filter cut-off may be set to 10 Hz and the sampling rate set to 40 Hz. For some applications, filter 34 comprises a band-pass filter having a low cutoff frequency between about 0.03 Hz and about 0.2 Hz, e.g., about 0.05 Hz, and a high cutoff frequency between about 1 Hz and about 10 Hz, e.g., about 5 Hz. Data acquisition module 20 typically digitizes the motion data at a sampling rate of at least 10 Hz, although lower frequencies are suitable for some applications.

Alternatively or additionally, the output of motion sensor 30 is channeled through several signal-conditioning channels, each with its own gain and filtering settings tuned according to the desired signal. For example, for breathing signals, a relatively low gain and a frequency passband of up to about 5 Hz may be used, while for heartbeat signals, a moderate gain and a slightly higher frequency cutoff of about 10 Hz may be used. For some applications, motion sensor 30 is additionally used for registration of acoustic signals, for which a frequency passband of about 100 Hz to about 8 kHz is useful.

In an embodiment of the present invention, system 10 is configured to monitor multiple clinical parameters of subject 12, such as respiration rate, heart rate, cough occurrence, body movement, deep inspirations, and/or expiration/inspiration ratio. Pattern analysis module 16 is configured to analyze the respective patterns in order to identify a change in the baseline pattern of the clinical parameters. In some cases, this change in the baseline pattern, which creates a new baseline substantially different from the previous baseline, is caused by a change in medication or other long-term change in the subject's condition, and provides the caregiver or healthcare professional with valuable feedback on the efficacy of treatment.

In an embodiment of the present invention, system 10 is configured to monitor clinical parameters, as defined hereinabove. Pattern analysis module 16 is configured to analyze the respective patterns in order to identify changes caused by medication and to provide feedback useful for optimizing the dosage of medication. For example, the medication may comprise a beta-blocker, which is used to treat high blood pressure (hypertension), congestive heart failure (CHF), abnormal heart rhythms (arrhythmias), and chest pain (angina), and sometimes to prevent recurrence of myocardial infarction (MI) in patients who have suffered a first MI. By measuring the heart rate patterns during sleep on a nightly basis, for example, the system may identify the effect of the medication, which may assist in adjusting the dosage until the optimal heart rate pattern is achieved. The system either reports the data to the patient or to the healthcare professional for use in adjusting the dosage, or transmits the data to an automatic drug dispensing device, which adapts the dosage accordingly.

Reference is again made to FIG. 1. In an embodiment of the present invention, motion sensor 30 comprises a pressure sensor (for example, a piezoelectric sensor) or an accelerometer, which is typically configured to be installed in, on, or under surface 37 upon which the subject lies, e.g., sleeps, and to sense breathing- and heartbeat-related motion of the subject. Typically, surface 37 comprises a mattress, a mattress covering, a sheet, a mattress pad, and/or a mattress cover. For some applications, motion sensor 30 is integrated into surface 37, e.g., into a mattress, and the motion sensor and reclining surface are provided together as an integrated unit. For some applications, motion sensor 30 is configured to be installed in, on, or under surface 37 in a vicinity of an abdomen 38 or chest 39 of subject 12. Alternatively or additionally, motion sensor 30 is installed in, on, or under surface 37 in a vicinity of a portion of subject 12 anatomically below a waist of the subject, such as in a vicinity of legs 40 of the subject. For some applications, such positioning provides a clearer pulse signal than positioning the sensor in a vicinity of abdomen 38 or chest 39 of the subject.

Reference is again made to FIG. 2. In an embodiment of the present invention, motion sensor 30 communicates wirelessly with control unit 14. In this embodiment, motion sensor 30 comprises or is coupled to a sensor wireless communication module 56, which wirelessly transmits and/or receives data to/from a control unit wireless communication module 58 that is coupled to control unit 14. The communications modules communicate using a signal that is analog (e.g., using standard AM or FM), or digital (e.g., using the Bluetooth® protocol). For example, in a hospital setting, a subject site such as a bed is typically occupied by each subject for only a few days. In some cases, it may be useful to replace sensor 30 whenever a new subject is assigned to the bed. In some cases, time spent by a nurse can be reduced by placing under a mattress a pad comprising sensor 30 and wireless communication module 56. The use of such a wirelessly-enabled sensor pad eliminates the need to connect and disconnect cables from control unit 14. Such use also makes the nurse's, physician's and subject's approach and/or entry into the bed more convenient. In embodiments in which sensor 30 operates wirelessly, the sensor, or a sensor assembly that comprises the sensor and the wireless communication module, typically comprises an internal power source, such as a battery. In order to preserve battery life, sensor 30 typically initiates communication upon detection of a relevant motion signal or other input.

In some settings, for example in hospitals, a plurality of systems 10 may be used in relatively close proximity. In such scenarios, each control unit 14 typically communicates only with the correct motion sensor 30 and not erroneously with another motion sensor 30 positioned at a different bed and associated with a different system 10. Bluetooth protocols, for example, allow for such pairing processes. In an embodiment, the system performs such pairing without initiating a conventional Bluetooth-type pairing process on both the sensor side and the control unit side. In addition to wirelessly-enabled motion sensor 30, control unit 14 is coupled to one or more contact sensors 60 applied to subject 12, such as a blood oxygen monitor 86 (e.g., a pulse oximeter), an ECG monitor 62, or a temperature sensor 80. Control unit 14 extracts pulse information from contact sensors 60. In order to identify the paired motion sensor 30 among several such transmitting motion sensors 30 within wireless range of the control unit, the control unit calculates the pulse data from each wireless signal received from a motion sensor 30 and identifies a signal that has pulse data that correlates with information received from contact sensors 60. Upon identifying such a match, the control unit records identifying features of the wireless communication module 56 coupled to the identified motion sensor 30 (e.g., a transmitter unique ID), such that from that point onward the identified sensor 30 is paired to control unit 14. For some applications, upon performing such pairing, control unit 14 notifies a healthcare worker that contact sensors 60 are no longer required and that the subject can be monitored with contactless sensor 30 only, or with fewer contact sensors 60.

For some wireless applications, upon activation of sensor 30, the nurse presses a connect button on control unit 14 and taps one or more times on sensor 30. Control unit 14 then connects to the one of a plurality of sensors 30 in the vicinity which transmits the taps at that exact point in time. Alternatively, user interface 24 provides a visual or audio indication of the taps, and the healthcare worker verifies that his or her taps are correctly displayed before approving the pairing of the sensor to the control unit. For some applications, the sensor, including the sensor plate, as described hereinbelow, does not comprise any buttons or other user controls. (These applications do not exclude the use of an on/off switch on wirelessly-enabled motion sensor 30.) For some applications, wirelessly-enabled motion sensor 30 is activated and paired with control unit 14 without requiring the pressing of any buttons or controls on the sensor. Instead the sensor is activated and paired either by tapping on the sensor or by temporarily connecting the sensor to the control unit with a wire. For some applications, a temporary cable is used to initiate the pairing of sensor 30 and control unit 14. After the sensor and control have been paired, the temporary cable is disconnected and the system operates using wireless communication. Alternatively or additionally, a motion sensor (e.g., a pressure sensor) coupled to control unit 14 by a wire is briefly placed on the reclining surface and pressed down against the mattress. The simultaneous readings from the wired motion sensor and from wirelessly-enabled motion sensor 30 enable control unit 14 to identify the particular wirelessly-enabled motion sensor 30 that is under the mattress that was pressed.

In an embodiment of the present invention, control unit 14 uses the pulse information provided by the contact sensor(s) to verify the accuracy of the respiration data monitored using motion sensor 30. Control unit 14 uses the information from sensor 30 to calculate respiration rate and heart rate and uses the information from the contact sensor to calculate heart rate. A correlation between the heart rate measured using the contact sensors and the heart rate measured using the sensor 30 indicates that the respiration calculated from sensor 30 is accurate as well.

In an embodiment of the present invention, sensor 30 is configured to operate during a limited period of time. For some applications, sensor 30 comprises an internal timer configured to measure the amount of time the sensor is both in use and communicating with control unit 14. After a predetermined period of active use, sensor 30 is configured to no longer communicate with any control unit 14. For some applications, each sensor 30 has a unique ID. A global database of used and non-used sensors is maintained. Upon connection to a new sensor unit 30, control unit 14 checks in the global sensor database whether the sensor has been used elsewhere. This global database, in some embodiments, also maintains general calibration and other useful data for the operation of control unit 14.

In an embodiment of the present invention, sensor 30 comprises a single piezoelectric ceramic sensor. The sensor is attached to a plate, e.g., a semi-rigid plate comprising flexible plastic (e.g. Perspex (PMMA)), or non-plastics (e.g., cardboard), for example having dimensions of 20 cm×28 cm×1.5 mm. The sensor is able to detect a signal when the subject assumes most common bed postures, even when the subject's body is not directly above the sensor.

For some applications, motion sensor 30 (for example, comprising a piezoelectric sensor) is encapsulated in a rigid compartment, which typically has a surface area of at least 10 cm², and a thickness of less than 5 mm. The sensor output is channeled to an electronic amplifier, such as a charge amplifier typically used with piezoelectric sensors, and capacitive transducers to condition the extremely high output impedance of the amplifier to a low impedance voltage suitable for transmission over long cables. The sensor and electronic amplifier translate the mechanical vibrations into electrical signals.

In an embodiment of the present invention, motion sensor 30 comprises a grid of multiple sensors, configured to be installed in, on, or under reclining surface 37. The use of such a grid, rather than a single unit, may improve breathing and heartbeat signal reception.

In an embodiment of the present invention, breathing pattern analysis module 22 extracts breathing-related signals by performing spectral filtering in the range of about 0.05 to about 0.8 Hz, and heartbeat pattern analysis module 23 extracts heartbeat-related signals by performing spectral filtering in the range of about 0.8 to about 5.0 Hz. For some applications, motion data acquisition module 20 adapts the spectral filtering based on the age of subject 12. For example, small children typically have higher breathing and heart rates, and therefore spectral filtering is typically set more tightly to the higher end of the frequency ranges, such as between about 0.1 and about 0.8 Hz for breathing, and between about 1.2 and about 5 Hz for heartbeat. For adults, spectral filtering is typically set more tightly to the lower end of the frequency ranges, such as between about 0.05 and about 0.5 Hz for breathing, and between about 0.5 and about 2.5 Hz for heartbeat.

In an embodiment of the present invention, pattern analysis module 16 derives a heartbeat signal from a breathing-related signal. This approach may be useful, for example, if the breathing-related signal is clearer than the directly monitored heartbeat signal. This sometimes occurs because the breathing-related signal is generated by more significant mechanical body movement than is the heartbeat-related signal.

In an embodiment of the present invention, the measured breathing-related signal is used to demodulate the heartbeat-related signal and thus enable improved detection of the heartbeat-related signal. Heartbeat pattern analysis module 23 demodulates the heartbeat-related signal using the breathing-related signal, such as by multiplying the heartbeat-related signal by the breathing-related signal. This demodulation creates a clearer demodulated signal of the heartbeat-related signal, thereby enabling its improved detection. In some cases, the power spectrum of the demodulated signal shows a clear peak corresponding to the demodulated heart rate. For some applications, the breathing-related signal used in the demodulation is filtered with a reduced top cut-off frequency (for example about 0.5 Hz, instead of the about 0.8 Hz mentioned above). Such a reduction generally ensures that only the basic sine wave shape of the breathing-related signal is used in the demodulation calculation.

In an embodiment of the present invention, for each of the filtered signals, a power spectrum is calculated and a largest peak is identified. A ratio of the heart rate-related peak to the respiration-related peak is calculated. The ratio is plotted for the duration of the night. This ratio is generally expected to remain constant for as long as the subject is lying in the same position. For each two consecutive time epochs (an epoch typically being between 30-300 seconds, for example 60 seconds), data acquisition module 20 calculates the percentage change of this ratio between the two epochs. The system determines that a change in body posture has occurred when the percentage change of the ratio is more than a threshold (typically between about 10% and about 50%, for example, about 25%). The frequency and timing of these changes is measured as an indication for restlessness in sleep.

In an embodiment, the change in the frequency distribution of the cardio-ballistic signal is used as an indication of a posture change.

Premature babies often need to be closely monitored at home or in the hospital to provide early warning of deterioration of their condition, because of infection, for example. In an embodiment of the present invention, system 10 is configured to closely monitor premature babies in a contactless manner, and to provide a warning to a parent or healthcare professional upon any change in the measured clinical parameters.

In an embodiment of the present invention, system 10 identifies a trend of change in one or more of the measured clinical parameters as an indication of the onset or progression of a clinical episode. For example, increases in respiration rate over three consecutive nights may indicate to system 10 that an asthma exacerbation is likely.

In an embodiment of the present invention, system 10 calculates an asthma score based on measured clinical parameters. For some applications, the system uses the following equation to calculate the asthma score:

$$S ( D ) = \frac{\begin{matrix}20 {R}_{a} ( D ) + 20 {R}^{′} ( D ) + 20 {R}_{b} ( D ) + 10 {HR}_{a} ( D ) + \\ 10 {HR}^{′} ( D ) + A C ( D ) + 5 SE ( D ) + 5 DI ( D )\end{matrix}}{N} \qquad ( Equation 1 )$$

wherein:

S(D)—asthma score for date D

Rₐ(D)—average respiration rate for date D, divided by the average respiration rate for all previous measured dates.

R′(D)—first derivative of the respiration rate calculated as follows:

$${R}^{′} ( D ) = \frac{R ( D ) - R ( D - 1 )}{R ( D - 1 )} \qquad ( Equation 2 )$$

wherein R(D) is the average respiration rate for date D and R(D−1) is the average respiration rate for the date immediately prior to date D.

R_{b}(D)—average respiration rate for the date immediately prior to date D, divided by the average respiration rate over the previous n dates, e.g., the previous three dates.

HRₐ(D)—average heart rate for date D, divided by the average heart rate for all previous measured dates.

HR'(D)—first derivative of the average heart rate calculated as follows:

$${HR}^{′} ( D ) = \frac{HR ( D ) - HR ( D - 1 )}{HR ( D - 1 )} \qquad ( Equation 3 )$$

wherein HR(D) is the average heart rate for date D and HR(D−1) is the average heart rate for the date immediately prior to date D.

AC(D)—a measure of activity level during sleep (restlessness) for date D, divided by the average of that measure for all previous measured dates.

SE(D)—sleep efficiency for date D, divided by the average sleep efficiency for all previous measured dates.

DI(D)—number of deep inspirations for that date D, divided by the average number of deep inspirations for all previous measured dates.

N—an integer dependent upon the condition under consideration, among other things, and typically having a value between about 80 and about 110, such as between about 88 to about 92, for example, about 91.

Each of the above-mentioned parameters is calculated for the duration of the sleep time or specific hours during the night prior to date D.

The values of Rₐ(D), HRₐ(D), AC(D), SE(D), and DI(D) are typically calculated for at least three dates prior to date D, for example, for at least three successive dates immediately prior to date D. Alternatively, Rₐ(D), HRₐ(D), AC(D), SE(D), and DI(D) are calculated as a ratio of the measurement of the current date to the average over K dates, wherein K is typically between about 7 and about 365, such as about 30. Alternatively, for some applications, the K dates are successive dates, for example, K successive dates immediately before date D. Alternatively, Rₐ(D), HRₐ(D), AC(D), SE(D), and DI(D) are calculated as ratios of the measurement of the current date to the average over the previous K nights that have not included an exacerbation of the chronic condition, identified either manually by user input, or automatically by system 10. For some applications, the average heart rate for each minute of sleep is calculated, and the standard deviation of this time series is calculated. This standard deviation is added as an additional parameter to, for example, a score equation such as Equation 1 above.

In an embodiment of the present invention, system 10 calculates the asthma score based on the clinical parameters, as defined hereinabove. For some applications, the equation comprises a linear expression of the clinical parameters, for example: the breathing rate change in percent versus baseline and the rate of coughs per a specific length of time. For some applications, the equation is an expression dependent on the clinical parameters that is close to linear, i.e., when the score is graphed versus any of the clinical parameters the area between the graph of the score and the closest linear approximation would be relatively small compared to the area under the linear approximation (e.g., the former area is less than 10% of the latter area). For some applications, the asthma score is calculated using the following equation:

S(D)=100−BR(D)−C(D) (Equation 4)

wherein:
S(D)—asthma score for date D.
BR(D)—percent increase in average respiration rate during sleep for date D vs. the subject's baseline (e.g., if respiration rate BR for date D is 20% above baseline, then BR(D)=20).
   C(D)—the number of cough events for date D (e.g., the number of coughs measured between 12:00 midnight and 6:00 AM or over another period), or the rate of cough events per unit time.

In an embodiment, the calculated asthma score is compared to a threshold (e.g., between about 50 and about 90, such as about 75). If the score is below the threshold, subject 12 or a healthcare worker is alerted that intervention is required.

In an embodiment of the present invention, system 10 calculates an asthma score based on the clinical parameters, as defined hereinabove. For some applications, the asthma score is calculated using the following equation:

S(D)=100−k1*BR(D)−k2*C(D) (Equation 5)

wherein:
S(D)—asthma score for date D.
BR(D)—percent increase in average respiration rate during sleep for date D vs. the subject's baseline (e.g., if respiration rate BR for date D is 20% above baseline, then BR(D)=20).
C(D)—the number of cough events for date D (e.g. the number of coughs measured between 12:00 midnight and 6:00 AM or over another period), or the rate of cough events per unit time.
k1, k2—coefficients for the respiration rate and cough parameters. Typically k1 and k2 are between about 0.7 and about 1.3.

In an embodiment, the calculated asthma score is compared to a threshold (e.g., between about 50 and about 90, such as about 75). If the score is below the threshold, the subject 12 or a healthcare worker is alerted that intervention is required.

In an embodiment of the present invention, system 10 calculates an asthma score based on the clinical parameters, as defined hereinabove. For some applications, the asthma score is calculated using the following equation:

S(D)=100−BR(D)−C(D)−RS(D) (Equation 6)

wherein:
S(D)—asthma score for date D.
BR(D)—percent increase in average respiration rate during sleep for date D vs. the subject's baseline (e.g., if respiration rate BR for date D is 20% above baseline, then BR(D)=20).
   C(D)—the number of cough events for date D. In an embodiment, this is measured between 12:00 midnight and 6:00 am, or over another period, or the rate of cough events per unit time.
   RS(D)—The level of restlessness in sleep for date D (e.g., on a scale of 0-Y, where typically Y is between 10 and 30, for example, 17, where Y is the highest level of restlessness and 0 is the lowest level).

In an embodiment, the calculated score is compared to a threshold (typically between about 60 and about 80, such as about 74). If the score is below the threshold, subject 12 or a healthcare worker is alerted that intervention is required.

As mentioned above, motion of the subject during sleep includes regular breathing-related and heartbeat-related movements as well as other unrelated body movements. In general, breathing-related motion is the dominant contributor to body motion during sleep. In an embodiment of the present invention, pattern analysis module 16 is configured to substantially eliminate the portion of the motion signal received from motion data acquisition module 20 that represents motion unrelated to breathing and heartbeat. For some applications, pattern analysis module 16 removes segments of the signal contaminated by non-breathing-related and non-heartbeat-related motion. While breathing-related and heartbeat-related motion is periodic, other motion is generally random and unpredictable. For some applications, pattern analysis module 16 eliminates the non-breathing-related and non-heartbeat-related motion using frequency-domain spectral analysis or time-domain regression analysis. Techniques for applying these analysis techniques will be evident to those skilled in art who have read the present application. For some applications, pattern analysis module 16 uses statistical methods, such as linear prediction or outlier analysis, to remove non-breathing-related and non-heartbeat-related motion from the signal.

In an embodiment of the present invention, pattern analysis module 16 determines the onset of an attack, and/or the severity of an attack in progress, by comparing the measured breathing rate pattern to a baseline breathing rate pattern, and/or the measured heart rate pattern to a baseline heart rate pattern.

In an embodiment of the present invention, pattern analysis module 16 comprises cough analysis module 26, which is configured to detect and/or to assess coughing episodes associated with approaching or occurring clinical episodes. In asthma, mild coughing is often an important early pre-episode marker indicating impending onset of a clinical asthma episode (see, for example, the above-mentioned article by Chang AB). In congestive heart failure (CHF), coughing may provide an early warning of fluid retention in the lungs caused by worsening of the heart failure or developing cardiovascular insufficiency.

For some applications, coughing sounds are extracted from motion sensor 30 installed in, on, or under a reclining surface, or from a microphone installed in proximity of the subject, typically using acoustic band filtering of between about 50 Hz and about 8 kHz, e.g., between about 100 Hz and about 1 kHz. Alternatively, the signal is filtered into two or more frequency bands, and motion data acquisition module 20 uses at least one frequency band of typically very low frequencies in the range of up to about 10 Hz for registering body movements, and at least one other frequency band of a higher frequency range, such as between about 50 Hz and about 8 kHz, for registering acoustic sound. For some applications, the module uses a narrower acoustic band, such as between about 150 Hz and about 1 kHz.

In an embodiment of the present invention, breathing pattern analysis module 22 is configured to detect, typically during night sleep, an abnormal breathing pattern associated with CHF, such as tachypnea, Cheyne-Stokes Respiration (CSR), or periodic breathing.

Patients with sleep apnea are often treated with Continuous Positive Airway Pressure (CPAP) systems. In many cases, it is beneficial to sense the respiration rate and heart rate in order to optimize the use of CPAP devices. In an embodiment of the present invention, the breathing-related signals and heartbeat-related signals which motion data acquisition module 20 extracts (as well as, in some cases, other clinical parameters measured by system 10) are used to optimize the operation of the CPAP device.

In an embodiment of the present invention, motion sensor 30 and all or a portion of motion data acquisition module 20 are packaged in a biocompatible housing (or in multiple housings) configured to be implanted in subject 12. The implantable components comprise a wireless transmitter, which is configured to transmit the acquired signals to an external receiver using a transmission technology such as RF (e.g., using the Bluetooth® or ZigBee protocols, or a proprietary protocol) or ultrasound. Alternatively, one or more of analysis modules 22, 23, 26, 28, 29, or 31, and/or user interface 24 are also configured to be implanted in subject 12, either in the same housing as the other implantable components, or in separate housings. Further alternatively, motion sensor 30 is configured to be implanted in subject 12, while motion data acquisition module 20 is configured to be external to the subject, and to communicate with motion sensor 30 either wirelessly or via wires.

In an embodiment of the present invention, system 10 comprises a plurality of motion sensors 30, such as a first sensor in a vicinity of abdomen 38 or chest 39 (FIG. 1), and a second sensor in a vicinity of legs 40. Pattern analysis module 16 determines a time delay between the pulse signal measured by the sensor under the abdomen or chest and the pulse signal measured by the sensor under the legs. For some applications, the module measures the time delay by performing a cross correlation between the heartbeat signals using a time window less than the respiration cycle time, such as between about 1 and 3 heart beat cycles. Alternatively, for some applications, the module identifies the peaks in the heartbeat signals, and calculates time differences between the signal peaks. Pattern analysis module 16 uses the time differences to calculate a blood pressure change signal on a continuous basis, for example as described in the above-mentioned U.S. Pat. No. 6,599,251 to Chen et al., mutatis mutandis. Module 16 calculates an amplitude of the change in the blood pressure change signal over a full inspiration/expiration cycle, and compares the amplitude to a threshold, such as 10 mmHg, or to a baseline value, either previously measured for the subject or based on a population average. Module 16 interprets amplitudes greater than the threshold as indicative of pulsus paradoxus. Alternatively or additionally, the system displays the amplitude and/or logs the amplitude to form a baseline for the specific subject which is later used to identify a change in condition.

In some cases, an increase in the average delay of the heart beat from the area of the heart to the extremities of the limbs is used as an indication of a deterioration in heart performance.

In an embodiment of the present invention, system 10 comprises one or more mechanical motion sensors as described above (e.g., a piezoelectric sensor) and a pulse oximeter sensor such as the OxiMax® sold by Nellcor of Pleasanton, Calif. The system measures a propagation delay between detection of a pulse signal detected by the mechanical sensor placed under the subject's chest area and detection of a pulse signal detected by the pulse oximeter sensor placed on the subject's finger. For some applications, the system measures this propagation delay using a cross-correlation calculation. The system outputs the delay to user interface 24 and/or logs the delay. In addition, changes in the delay are used as described above for evaluating change in blood pressure, change in cardiac output and detection of pulsus paradoxus. For some applications, the propagation delay is used as one of the clinical parameters, as defined hereinabove, such as for calculating the subject's score. In an embodiment, pulse propagation is detected using a contactless sensor.

In an embodiment of the present invention, the system uses the propagation delay described immediately above to calculate blood pressure, for example using the pulse transit time method described in the above-mentioned article by Sorvoja, H. and Myllylä, R. for identifying changes in blood pressure. For some applications, system 10 identifies body movements as described herein and identifies transit time changes that are correlated with body movements as false alarms.

In some embodiments of the present application, the system identifies and provides an alert upon detecting a significant change in blood pressure, for example a drop in systolic blood pressure that is considered a warning sign that requires medical intervention, such as for hospitalized subjects.

In some cases, a pulse oximeter may give erroneous readings without any visible warning. This may happen, for example, because of poor perfusion. In an embodiment of the present invention, system 10 comprises the above-mentioned pulse oximeter and a mechanical sensor. System 10 calculates the subject's heart rate using both the pulse oximeter signal and the mechanical sensor's signal. The system compares the two calculated heart rates to verify that the measured heart rate is correct. If there is a mismatch, the system alerts a healthcare worker.

The pulse signal detected by the pulse oximeter is modulated by the subject's respiration cycle. In an embodiment of the present invention, system 10 uses the level of modulation of the pulse signal detected in the pulse oximeter during a respiratory cycle to evaluate whether the subject suffers from pulsus paradoxus. For some applications, in order to identify this modulation, the system measures the respiratory signal using the mechanical sensor described above. The system analyzes the signal to find the frequency and timing of the respiratory cycle, and, accordingly, to measure the depth of the modulation of the pulse signal by the respiratory cycle. For some applications, the system uses a technique similar to that described in U.S. Pat. No. 5,743,263 to Baker, mutatis mutandis, except that the respiration rate, instead of the heart rate, is used as a virtual trigger.

In an embodiment of the present invention, system 10 uses the heart rate as detected by a contactless mechanical sensor as described hereinabove in order to improve the signal-to-noise ratio in the pulse oximeter reading. For example, the heart rate is used as a virtual trigger in a similar manner to the technique described in U.S. Pat. No. 5,743,263 to Baker. Alternatively, the exact timing of the pulse signal as measured by the contactless mechanical sensor is used to trigger the heart beat synchronization process, in order to improve the signal-to-noise ratio in the pulse oximeter signal.

In an embodiment of the present invention, system 10 is configured to monitor breathing and pulse (or heartbeat) patterns in order to recognize Central Sleep Apnea (CSA) episodes.

In an embodiment, system 10 comprises a Positive Airway Pressure (PAP) device. Upon detecting that the subject has fallen asleep, the system activates the PAP device. Alternatively, the system activates the PAP device a predefined period of time after the system identifies quiet breathing, so as to facilitate the falling asleep of the subject, which may be compromised by the activation of PAP. For some applications, techniques of this embodiment are used to treat a subject suffering from obstructive sleep apnea (OSA), without preventing the subject from falling asleep.

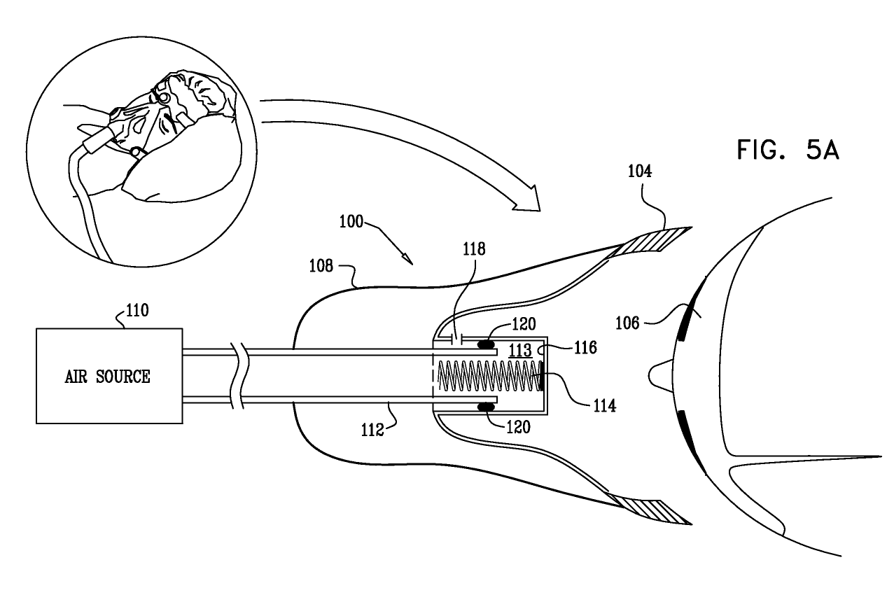

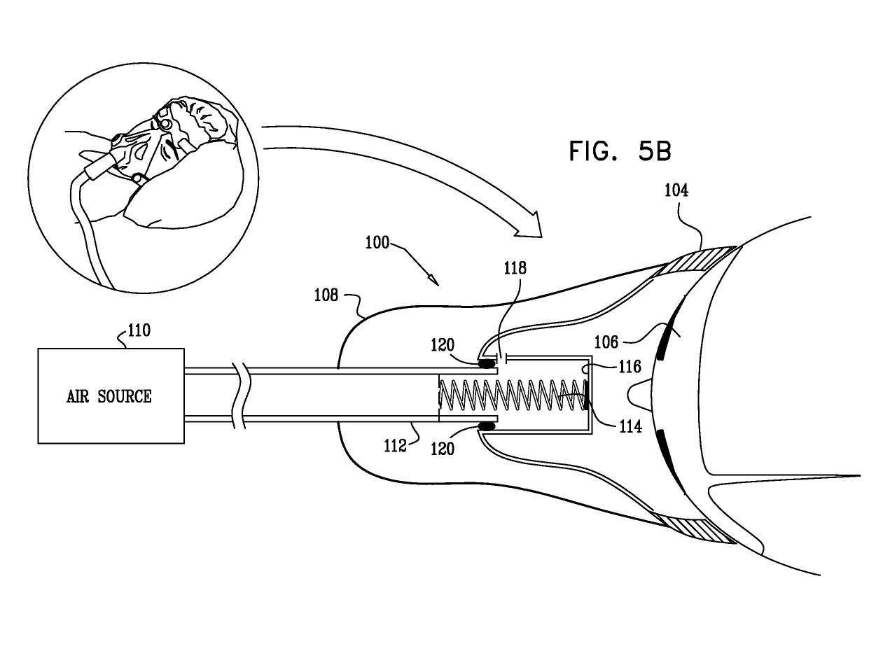

Reference is made to FIGS. 5A-B and 6A-B, which are schematic illustrations of a positive airway pressure (PAP) device 100 and a PAP device 102, respectively, in accordance with respective embodiments of the present invention. In these embodiments, system 10 controls PAP device 100 or PAP device 102 to selectively activate the device to apply PAP, or to facilitate normal breathing by the subject. For some applications, when PAP is not required, system 10 opens one or more windows or vent holes in a mask 104 of PAP device 100 or PAP device 102, in order to facilitate normal breathing by the subject, for example so as to make falling asleep easier for the subject. Subsequently, when system 10 detects that PAP is needed, the system closes or minimizes the size of the window(s) in the mask in order to enable the device to deliver positive airway pressure to the subject's airways.

FIGS. 5A and 5B show PAP device 100 in inactive and active states, respectively. In the inactive state shown in FIG. 5A, mask 104 is held at a distance from a face 106 of the subject by a retaining mechanism 108, which comprises, for example, semi-rigid headgear. Upon detection that PAP is required, system 10 drives an air source 110 to apply air pressure to the mask via an air delivery tube 112, a distal end of which is positioned within a tubular cavity 113 of the mask. The pressure causes expansion of a spring 114 positioned between retaining mechanism 108 (e.g., headgear) and mask 104, such as a surface 116 of cavity 113 of the mask that faces the spring and the distal end of the tube. Expansion of the spring pushes mask 104 via surface 116 into contact with face 106, as shown in FIG. 5B. The movement of mask 104 with respect to the distal end of tube 112 unblocks a vent hole 118 of the mask, so air supplied by air source 110 flows into the mask. An o-ring 120 is positioned between an outer surface of the distal end of tube 112 and the wall of cavity 113, to prevent air from entering vent hole 118 when PAP device 100 is in its inactive state, as shown in FIG. 5A, and to prevent air from leaking out of cavity 113 when PAP device 100 is in its active state, as shown in FIG. 5B.

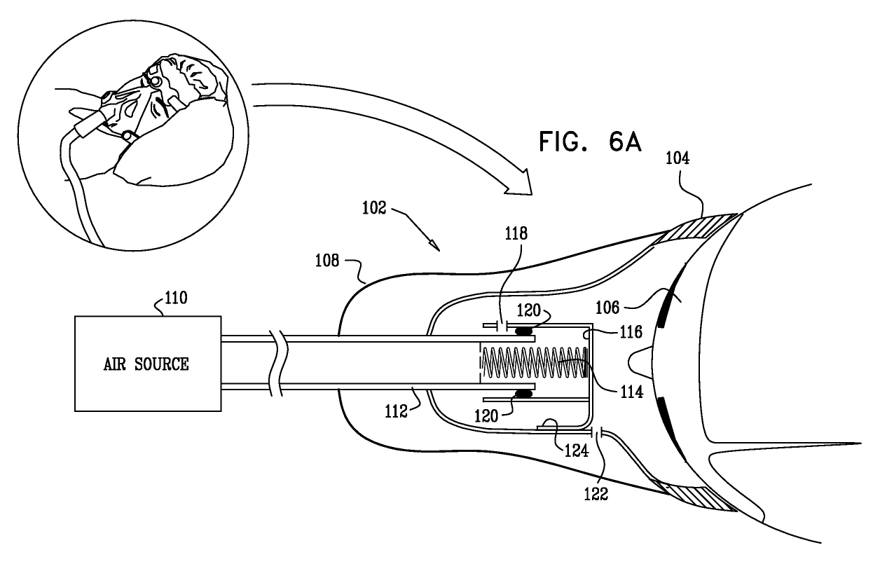

FIGS. 6A and 6B show PAP device 102 in inactive and active states, respectively. In this embodiment, mask 104 is held in contact with face 106 even when PAP device 102 is in its inactive state. PAP device 102 thus does not necessarily comprise retaining mechanism 108 to hold the mask. When PAP is not required, for example when the system detects that the subject is awake, or when the system does not detect any apnea events, the system keeps mask vents 122 open to facilitate normal and comfortable breathing by the subject, as shown in FIG. 6A. Upon detecting that PAP is required, system 10 activates air source 110, which expands spring 114, pushing a covering element 124 over mask vents 122, and opening vent hole 118, through with PAP is delivered into mask 104 and through it to the subject's airways.

In an embodiment of the present invention, system 10 comprises a robotic arm that places a mask on the face of the subject when the system determines PAP is needed, and removes the mask when PAP is not needed.

Some subjects are at higher risk of sleep apnea during REM sleep than during other sleep stages. In an embodiment of the present invention, system 10 identifies when the subject enters REM sleep, such as described hereinbelow, and activates the PAP device responsively to the identification. Alternatively, system 10 adjusts one or more thresholds for activation or the PAP parameters upon detection of REM sleep.

In an embodiment of the present invention, system 10 provides therapy to prevent central sleep apnea by providing nerve simulation to prevent the central apnea. For some applications, system 10 uses techniques described in U.S. Pat. No. 5,540,734 to Zabara, which is incorporated herein by reference. For other applications, system 10 activates the nerve stimulation upon detection of the onset of sleep apnea episodes.

In an embodiment of the present invention, system 10 continuously monitors the heart rate of subject 12 during sleep. The system identifies and logs short-term increases in heart rate, and/or alerts a healthcare worker. For example, pattern analysis module 16 calculates average heart rate for each minute and the average for the previous 10 minutes. The system identifies the occurrence of an event upon detecting that the average heart rate in the current minute is at least a certain percent greater than the average of the previous 10 minutes, e.g., between about 5% and about 30%, such as about 10%. The system logs the number and severity of such events, and uses the events as an additional clinical parameter, as defined hereinabove. For example, such events may indicate a change in blood oxygen saturation level. Alternatively, the number and severity of such events is logged for a COPD subject and a significant change is used as an indication of a change in the subject's clinical condition. For some applications, system 10 builds a baseline of the characteristics of such peaks or troughs in heart rate for a subject over one or more nights, and alerts the subject or a healthcare worker upon detecting a clear change in the characteristics of such peaks, e.g., the height, frequency or distribution over the sleep period.

In an embodiment of the present invention, system 10 is configured to receive a specified range of values for a clinical parameter, such as heart rate or respiration rate. Responsively to motion sensed with motion sensor 30, the system calculates a value of the clinical parameter of the subject at least once every 10 seconds, during a period having a duration of at least 30 seconds, e.g., at least 60 seconds, or at least one hour. Only upon finding that the value falls outside the specified range over 50% of the times it is calculated throughout the period, the system generates an alert. For some applications, this technique is used to monitor subjects having a condition other than apnea or SIDS.

In an embodiment of the present invention, system 10 is configured to receive a specified range of values for a clinical parameter, such as heart rate or respiration rate. Responsively to motion sensed with motion sensor 30, the system calculates respective raw values of the clinical parameter of the subject at least once every 10 seconds, during a period having a duration of at least 30 seconds, e.g., at least 60 seconds, or at least one hour. The system calculates a representative value based on the raw values, such as a mean or median of the raw values, or another representative value based on the raw values (e.g., including discarding outlying raw values). Only upon finding that the representative value falls outside the specified range, the system generates an alert.

In an embodiment of the present invention, system 10 is configured to receive an indication of a baseline value for a clinical parameter, such as heart rate or respiration rate. Responsively to motion sensed with motion sensor 30, the system calculates a value of the clinical parameter of the subject at least three times, e.g., at least 10 times, during a period having a duration of at least 10 seconds, e.g., at least 30 seconds, at least 60 seconds, or at least one hour. Only upon finding that the value is at least a threshold percentage different from the baseline value over 50% of the times it is calculated throughout the period, the system generates an alert. For some applications, this technique is used to monitor subjects having a condition other than apnea or SIDS.

In an embodiment of the present invention, system 10 is configured to receive an indication of a baseline value for a clinical parameter, such as heart rate or respiration rate. Responsively to motion sensed with motion sensor 30, the system calculates respective raw values of the clinical parameter of the subject at least times, during a period having a duration of at least 10 seconds, e.g., at least 60 seconds, or at least one hour. The system calculates a representative value based on the raw values, such as a mean or median of the raw values, or another representative value based on the raw values (e.g., including discarding outlying raw values). Only upon finding that the representative value is at least a threshold percentage different from the baseline value, the system generates an alert.

Subjects undergoing cytotoxic chemotherapy are at high risk of suffering from CHF and/or pulmonary edema. In an embodiment of the present invention, system 10 is used to monitor subject 12 during and after receiving chemotherapy treatment and to alert the subject or a healthcare worker upon detection of a clinical indication of impending CHF or pulmonary edema.

In an embodiment of the present invention, system 10 is used to monitor subjects suffering from renal failure. System 10 identifies changes in vital signs (e.g. increase in heart rate and respiration rate or reduction in sleep quality) that indicate that a subject may need dialysis treatment or other intervention.

Pulmonary hypertension is characterized by elevated blood pressure in the pulmonary arteries from constriction in the lung or stenosis of the mitral valve. The condition adversely affects the blood flow in the lungs, and causes the heart to work harder. In an embodiment of the present invention, system 10 is used to monitor subjects suffering from pulmonary hypertension and to identify the onset and/or deterioration of their condition. System 10 monitors the clinical parameters and identifies a change that may indicate such a deterioration, for example an increase in respiration rate or heart rate.

Reference is again made to FIG. 2. In an embodiment of the present invention, system 10 uses a Bayesian classifier of acoustic and motion events in order to effectively identify cough events. Each event is parameterized by a set of parameters that forms the feature vector of the event. These parameters are derived from both motion and audio signals generated by a mechanical sensor (e.g., motion sensor 30, which may comprise, for example, a piezoelectric sensor placed under a mattress pad) and an acoustic sensor 82, e.g., a microphone, respectively. The system calculates these parameters in time and frequency domains. These parameters include, for example, the length in time of the event, the average acoustic frequency, a trend of change of the frequency along the event, and the standard deviation of the mechanical signal during the event. In addition, these parameters may include the results of an autoregressive model of the acoustic signal. The autoregression is performed with, for example, between about 3 and about 11 coefficients (e.g., about 5 coefficients). For some applications, final prediction error (FPE) is used as a parameter, as well as the height and width of the peak of FPE in the first phase of the cough, and the ratio of the height of successive peaks in FPE. For some applications, the system performs for each event a detection algorithm that is based on the following assumptions:
each acoustic event belongs to a specific class from a prescribed finite set of classes;
for each class from the prescribed set, the probability that a specific event belongs to the class is defined by an event feature vector; and
the probability density function (PDF) of each class is modeled by a Gaussian Mixture Model (GMM).

In an embodiment of the present invention, the system uses more than one type of classes. For some applications, the system uses exactly two classes: “cough” and “non-cough.” For other applications, the system uses more than two classes, for example: “cough,” “snore,” “cry,” and “other.” The parameterization of the PDF for each specific class is obtained through a learning process using a database of events with known classifications. Typically, a portion of the database is used as input data for the learning algorithm that calculates the PDF parameters (for example, an Expectation-Maximization algorithm). Another portion of the database is used as a test set for checking the detection algorithm.

In an embodiment of the present invention, system 10 monitors respiratory rate and identifies respiratory depression as a significant decrease in respiration rate compared to baseline. Upon detection of respiratory depression, the system records the event and optionally generates an alarm via user interface module 24. For some applications, the system is used for monitoring post-operative subjects, or subjects who have been treated with opioids, barbiturates, or other pain-relief drugs. In some instances, the use of such a monitoring system to detect and alarm upon respiratory depression enables the clinician to use such drugs where otherwise they would not be used. In other cases, it enables the clinician to increase the dosage of these drugs.

In an embodiment, system 10 detects changes in respiration rate, heart rate, and body motion that indicate that the subject is suffering from pain. For some applications, upon detection of pain, the system activates a drug administration device 84 (FIG. 2) in order to alleviate the pain automatically with the appropriate medication.

Reference is again made to FIG. 2. In an embodiment of the present invention, system 10 comprises a blood oxygen monitor 86 (e.g., a pulse oximeter). System 10 monitors a respiration pattern of the subject, a heart rate pattern of the subject, or a respiration motion pattern of the subject (which includes the depth of each breath) (or a combination of two or more of these patterns) while monitoring the subject's blood oxygen level using blood oxygen monitor 86. The system uses learning techniques to identify one or more characteristic patterns associated with an impending change in the blood oxygen level. Upon detecting at least one of the learned characteristic patterns that precede changes in blood oxygen level, the system generates an alert to the subject or a healthcare worker. The system thus serves as an early warning system for change in blood oxygen level. In some cases the changes in a heart rate pattern, a respiration rate pattern, and/or a respiration motion pattern precede the changes in blood oxygen level. Optionally, even when not performing learning, the system uses this pattern-monitoring technique in combination with blood oxygen monitor 86 in order to provide an earlier warning of an impending change in blood oxygen than is possible using the blood oxygen level meter alone. For some applications, the system uses blood oxygen monitor 86 only for learning the characteristic respiration or heart rate patterns, and not during subsequent monitoring of the subject for an impending change in blood oxygen level.

For some applications, system 10 interprets a change in respiratory rate and a change in respiratory pattern as indicative of a high probability of an impending deterioration in blood oxygen level. For example, an increased respiratory rate combined with shallow breaths in a resting patient may provide such an indication. An increased heart rate in conjunction with these changes serves as an additional indication of a high likelihood of a decline in oxygen saturation.

In an embodiment of the present invention, system 10 combines the information regarding blood oxygen measured using blood oxygen monitor 86 with information regarding respiration rate and/or heart rate measured using motion sensor 30, to generate a combined clinical score. When the score crosses a threshold, the system generates an alert that the subject is at risk of respiratory depression. For some applications, system 10 also calculates a clinical parameter of breathing irregularity. For some applications, the system calculates a baseline for the subject for each of the measured parameters over a baseline period of time (e.g., less than an hour, such between about 15 and about 45 minutes, or more than about an hour). The system calculates the clinical score using, for example, the following equation:

S=5(100−Ox)−DeltaRR−DeltaHR+RESPIrreg (Equation 7)

wherein:
S—clinical score
Ox—blood oxygen saturation level in percent
DeltaRR—percentage change in respiration rate versus baseline
DeltaHR—percentage change in heart rate versus baseline
RESPIrreg—percentage change in respiration irregularity versus baseline. The system may calculate the respiration irregularity, for example, using parameters BRSTD, STDP2P, MB2BC, or STDB2BC as defined hereinbelow. The relevance of respiration irregularity to respiratory depression is suggested in the above-mentioned article by Bouillon T., et al.

The system calculates each of these parameters substantially continuously during monitoring. If the calculated score crosses a threshold (e.g., 25), the system alerts the subject or a healthcare worker.

In an embodiment of the present invention, system 10 comprises blood oxygen monitor 86 and sensor 30. The system finds that the subject may be experiencing a deterioration of a condition, for example, asthma, responsively to detecting both (a) an increase in motion of the subject (i.e., restlessness) measured using sensor 30 and (b) a significant drop in blood oxygen level measured using blood oxygen monitor 86. Alternatively or additionally, if the system detects a drop in blood oxygen level during REM sleep, especially during the longer REM periods towards early morning, the system logs and analyzes the drop, which may indicate to a healthcare worker that the subject's condition, for example asthma, is deteriorating. For some applications, the system detects REM sleep using techniques described hereinbelow with reference to FIG. 11.

Reference is again made to FIG. 2. In an embodiment of the present invention, system 10 performs cough monitoring. The system measures the number of cough events during the monitoring period and the time of each cough occurrence. In an embodiment, system 10 detects coughing using acoustic sensor 82, which detects ambient audio signals in the vicinity of subject 12, for example, by sensing an audio signal near the subject, such as by placing a microphone within 100 cm of the subject. The system digitally analyzes the signal recorded from acoustic sensor 82, and identifies acoustical events that are greater than the background noise level. System 10 distinguishes between cough and non-cough acoustical events, such as by identifying acoustic signal patterns specific for coughs, and/or using techniques described hereinbelow or in one or more of the patent applications incorporated by reference hereinbelow. The non-cough acoustical events include, for example, human-generated sounds such as speech, laughing, or sneezing, mechanical high amplitude impulse-like noise, TV, and radio.

In an embodiment of the present invention, the system selects the time intervals that include acoustical events using signal energy and amplitude thresholds. The system calculates thresholds per a constant length segment of the acoustical record, wherein each segment includes a number of events and noise intervals. The segment is divided to windows of fixed small length. For some applications, the windows do not overlap, while for other applications, the windows overlap. For each window, the system calculates signal energy and maximum amplitude and obtains corresponding distributions of their values. The system extracts thresholds from these distributions taking into account typical tail considerations. Windows for which the values calculated are higher than the thresholds are united in intervals with acoustical events. The system rejects intervals that are shorter than or longer than the typical length of cough acoustic phases, or having a small number of amplitudes over threshold in comparison with the number of global maxima in the considered interval.

In an embodiment of the present invention, in order to detect a cough, the system first rejects signals that are identified as vocal or that have a length that is shorter or longer than thresholds, and subsequently examines the specific frequency change pattern that is indicative of a cough.

The above-mentioned article by Thorpe C et al., describes a three-phase cough structure, including an initial glottal opening burst (phase 1), a quieter middle phase (phase 2), and (sometimes) a final closing burst (phase 3).

In an embodiment of the present invention, the system detects cough envelopes using the envelope of the acoustical signal in the time domain. The form of the cough event envelope depends on the presence of phase 3 of the cough structure. If only phases 1 and 2 of the cough structure are present, the envelope has a specific geometry including a single maximum. If all three phases are present, the envelope has two-hump geometry.

In an embodiment of the present invention, the system detects cough envelopes by calculating the number and location of intersection points between the above-mentioned envelope and least mean square polynomial estimation of that envelope. Alternatively, the system applies a dynamic time warping algorithm to test the envelope.

In an embodiment of the present invention, the system calculates specific patterns that characterize non-cough acoustical events using frequencies related to signal amplitude zero-crossing points and time-frequency autoregressive characteristic(s) calculated using an autoregressive model of the acoustic signal, as described above with reference to FIG. 2 in the paragraph describing the Bayesian classifier of acoustic and motion events. For some applications, the pattern that distinguishes vocal, i.e., non-cough acoustical events, from cough events is the concentration of frequencies around a small (e.g., between one and four) number of fixed values. Upon identifying this pattern (e.g., using either zero-crossing and/or autoregressive methods), the system considers the event as vocal rather than a cough.

In an embodiment of the present invention, the system uses maximum/minimum detection instead of zero-crossing frequency calculation. Alternatively, the system uses a combination maximum, minimum and zero-crossing analysis in order to smooth the resulting frequency distribution.

In an embodiment of the present invention, the system detects an acoustic signature for coughs that differs for coughs with fluids in the lungs (pulmonary edema) and for cough without fluids in the lungs (normal condition). This distinction enables earlier warning for deterioration of congestive heart failure. For some applications, the system detects a cough signature that is different for a smoking person from that of a non-smoking person.

In some cases, especially when the heart rate is relatively low, higher harmonics of the respiration rate may appear in the spectrum of the heart channel and may affect the measurement of the heart rate. In an embodiment of the present invention, system 10 uses a band pass filter to eliminate most of the respiratory harmonics (as well as the basic frequency of the heart rate), using, for example, a pass band of between about 2 Hz and about 10 Hz. In a Fourier analysis of the resulting signal, the basic frequency of the heart rate is no longer the highest peak. However, the harmonics of the heart rate signal are still present as peaks. Heart beat pattern analysis module 23 identifies these peaks and calculates the heart rate by calculating the distance between consecutive peaks.

In another embodiment, system 10 calculates the heart rate using an amplitude demodulation method. In this method, a band pass filter which rejects the basic heart rate frequency as well as most of the respiratory harmonics is used. For example, the band pass filter may be tuned to between about 2 Hz and about 10 Hz. The absolute value of the filtered signal is calculated, and a low pass filter with appropriate cutoff frequency (e.g., about 3 Hz) is applied to the absolute value signal result. Finally, the system calculates the power spectrum and identifies its main peak, which corresponds to the heart rate.

### Tremor Measurements

There are multiple clinical uses for the measurement of tremor. One application is the monitoring of diabetic subjects to identify hypoglycemia. In an embodiment of the present invention, system 10 identifies the signal associated with heart rate and respiration rate. The system subtracts the heart rate and respiration rate signal from the overall signal. The resulting signal in those areas where there are no restlessness events is regarded as the tremor signal for the analysis described above. For some applications, the energy of the tremor signal is normalized by the size of the respiration and/or heart signal.

Typically, tremor-related oscillations occur in a frequency band of between about 3 and about 18 Hz. In an embodiment of the present invention, motion data acquisition module 20 and pattern analysis module 16 are configured to digitize and analyze data at these frequencies. The system attributes a significant change in the energy measured in this frequency range to a change in the level of tremor, and a change in the spectrum of the signal to a change in the spectrum of the tremor.

### CHF Deterioration, Edemas and Subject Weighing

Congestive Heart Failure (CHF) deterioration is often characterized by abnormal fluid retention, which generally results in swelling (edema) in the feet and legs. This edema is often diagnosed by having subjects weigh themselves daily and note a weight increase of over 1 kg in 24 hours. This diagnostic technique requires subject compliance with a daily weighing routine. In an embodiment of the present invention, system 10 is configured to identify a change in weight of subject 12. In an embodiment, sensor 30 includes a vibration sensor which is AC coupled (i.e., includes a high pass filter, for example, at 0.05 Hz), as well as a pressure sensor which is DC coupled (i.e., no high pass filter implemented). Optionally, both the vibration sensor and the pressure sensor are implemented using a single sensing component. The amplitude of the signal captured by the pressure sensor is proportional to the subject's weight (hereinbelow, the “weight signal”), and also depends on the subject's location and posture with respect to the sensor. The amplitude of the heart beat related signal captured by the vibration sensor (hereinbelow, the “heartbeat signal”) depends on the subject's posture and position as well as the strength of the cardioballistic effect. As fluids build up in the body, the subject's weight increases and the cardioballistic effect is reduced.

In an embodiment of the present invention, sensor 30 is placed under an area of the subject's legs. In this area body mass increases during events of edema, resulting in a reduced cardioballistic effect and an increased pressure due to body weight. Pattern analysis module 16 monitors a ratio of the weight signal to the heartbeat signal, and calculates a baseline value for the ratio. Upon detecting an increase in the ratio above baseline, which may indicate the onset of edema, system 10 notifies the subject and/or a healthcare professional, and/or integrates the change into the clinical score calculated by system 10. In an embodiment, the system averages this signal over a substantial portion of the night, such as in order to minimize the effects of a specific body posture and/or position.

At the onset of deterioration, CHF patients often sleep with their heads and lungs elevated with respect to the rest of their bodies. In an embodiment of the present invention, system 10 detects this elevation in order to provide an early indication of CHF deterioration. For some applications, multiple sensors 30 are placed under the mattress. The system identifies a change in the elevation and angle of about the top third of the body of subject 12, by detecting a change in the pressure distribution between the multiple sensors. For some applications, system 10 comprises a tilt sensor, which is placed on an external surface of the body of subject 12 in a vicinity of the lungs, or on the mattress or in a pillow subject 12 uses. For example, pattern analysis module 16 may interpret an increase in the subject's tilt angle during sleep compared to a baseline value measured on one or more previous nights as an indication of CHF deterioration. The system typically notifies the subject and/or a healthcare worker of the detected deterioration and/or integrates an indication of the deterioration into the subject's clinical score, as described hereinabove.

In an embodiment of the present invention, sensor 30 is configured to cover the entire area of the mattress, and system 10 is configured to measure the weight of subject 12 responsively to the sensor signal. For some applications, sensor 30 comprises a flexible chamber configured to contain a fluid, for example, a liquid or gas. The flexible chamber is configured to cover substantially the entire area of the mattress, such that it is deformed by pressure exerted on the mattress by subject 12. The sensor detects the pressure in the fluid in the chamber. The pressure increases with an increase in the weight of subject 12.

Cheyne Stokes Respiration (CSR) and Periodic Breathing (PB) are often indicators of deterioration of CHF. In an embodiment of the present invention, pattern analysis module 16 is configured to identify and measure the intensity of CSR and PB as indicators of a CHF condition.

In an embodiment of the present invention, system 10 comprises a plurality of sensors, for example, a plurality of weight sensing sensors, placed under the mattress or mattress pad upon which subject 12 rests. The system calculates a change in a ratio of the average weight sensed by the sensors. Such a change in the weight ratio may indicate that subject 12 has changed posture, for example, changed the angle of inclination during sleep. A change in the sleep angle may indicate that a subject who suffers from CHF or another physiological ailment, is beginning to feel decompensated. For some applications, the system integrates this weight change into the clinical score and/or outputs it to the subject and/or a healthcare worker.

### Insomnia

In an embodiment of the present invention, system 10 is configured to monitor a subject 12 who suffers from or is suspected of suffering from insomnia. For example, system 10 may monitor the duration subject 12 is in bed before falling asleep, the total duration of quiet sleep, a number of awakenings during sleep, sleep efficiency, and/or REM sleep duration and timing. The system calculates an insomnia score, for example, using one or more of the parameters used in the asthma score described hereinabove, and presents the score to the subject or a healthcare worker. For some applications, system 10 is used to evaluate the effectiveness of different therapies to treat insomnia and the improvement that is achieved by therapy, by comparing the sleep quality parameters before and after treatment. For some applications, system 10 detects the worsening of insomnia and outputs an indication that a change in therapy or additional therapy may be required. For some applications, system 10 automatically activates or administers a therapy to treat insomnia when the sensors and analysis of system 10 deem such therapy appropriate.

In an embodiment of the present invention, upon identifying the onset of an episode of apnea or other physiological event, system 10 applies an appropriate treatment or therapy automatically, such as continuous positive airway pressure (CPAP) or a change in body position (e.g., by inflating a pillow). For example, upon detecting or predicting the onset of an episode of apnea or another physiological event, system 10 may activate or administer an appropriate treatment or therapy within a short period of time (i.e., within seconds or minutes, e.g., less than five minutes, such as less than one minute). For some applications, system 10 activates a device configured to change the body and/or head position of subject 12, for example, so as to open up the airway in obstructive sleep apnea. For example, system 10 may include an inflatable pillow on which the subject sleeps, which, when activated, inflates or deflates to vary the elevation of the head of subject 12 as desired. Upon detecting or predicting an episode of apnea or another physiological event, the system changes the pillow's air pressure level in order to change the subject's posture and prevent and/or stop the physiological event.

### Alcohol Withdrawal

In an embodiment of the present invention, system 10 is used to monitor subjects in a home, hospital or long term care facility. For some applications, system 10 monitors subjects who are at risk of alcohol withdrawal. Upon identifying early warning signs of alcohol withdrawal such as tachycardia, palpitations, tremor, agitation in sleep, or seizures, the system alerts the subject, a family member, or a healthcare worker to provide appropriate intervention.

### Pulmonary Edema

In an embodiment of the present invention, system 10 detects and calculates the amplitude of a heartbeat-related signal, the amplitude of a tremor signal, and a ratio of the heart-beat-related signal amplitude to the tremor signal amplitude. The system interprets a change in the average ratio of these signals as an indicator of pulmonary edema. For example, the system may interpret a decrease in the ratio of more than a certain percentage (e.g., 10%) as indicative of the onset of edema. For some applications, the system averages the ratios over the entire night. Alternatively, the system averages the ratio over less than an hour (e.g., several minutes), or more than an hour. For some applications, the sensor is located under the area of the legs or the chest where edema is expected to occur in heart failure subjects.

In an embodiment of the present invention, the system interprets changes in these parameters as an indication of a change in temperature of the legs, which is indicative of a change in condition of a diabetic subject.

In an embodiment of the present invention, system 10 is used to monitor a subject while in a hospital. After the subject is released from the hospital to his or her home or a long-term care facility, the same or a similar system is used for monitoring, such that the data acquired during hospitalization is available as reference for the system in the home/long-term phase of treatment. Furthermore, if the subject is readmitted to the hospital, the data from the home/long-term phase is available to the hospital system. For example, the hospital system may use such home/long-term data to determine when the subject's clinical score or specific clinical parameter has returned to within a specific range from the baseline measured at home/long-term care. Upon detecting such a return towards baseline, the system outputs an indication that, for example, the subject may be sent home or to long-term care.

In an embodiment of the present invention, system 10 is used for monitoring subjects who are in the process of being weaned off a respiratory machine or oxygen support. The system detects the respiratory patterns and additional clinical parameters of such subjects, and identifies changes in order to detect any improvement or deterioration in the subject's condition and to alert accordingly.

In an embodiment of the present invention, system 10 is configured to detect the onset or the early warning signs of febrile convulsions or febrile fits. Febrile convulsions occur in young children when there is a rapid increase in their body temperature. For some applications, system 10 identifies an increase in body tremor, heart rate, palpitations, or respiration rate and provides an early indication of febrile convulsions. In another embodiment, system 10 identifies the actual febrile convulsion and provides an indication and a log of all such events for clinicians.

In an embodiment of the present invention, system 10 is used to monitor a subject 12 who is undergoing a lung transplant. The system monitors the subject on a daily basis and identifies a trend. If the system identifies a change in a clinical score that may indicate deterioration of the subject's condition, the system alerts the subject or a healthcare worker. For example, an increase in respiratory rate in sleep versus previous nights may indicate that the subject is beginning to reject the lung transplant.

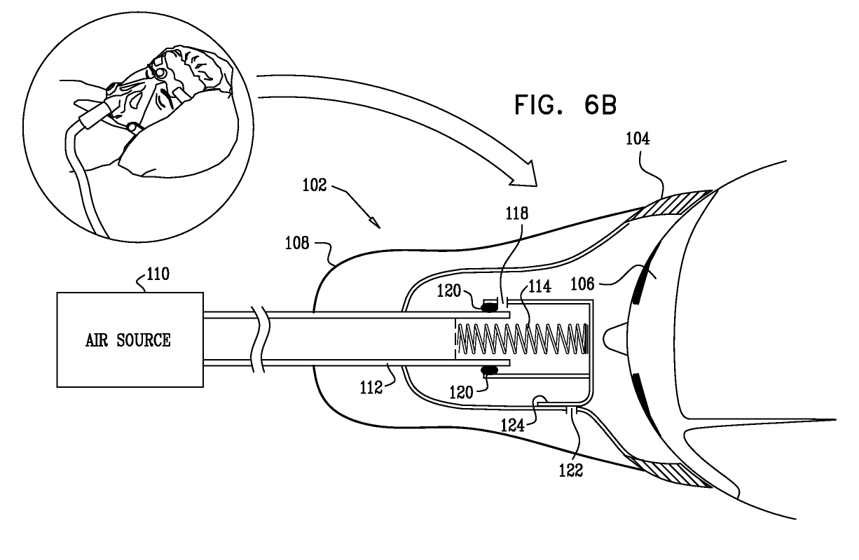

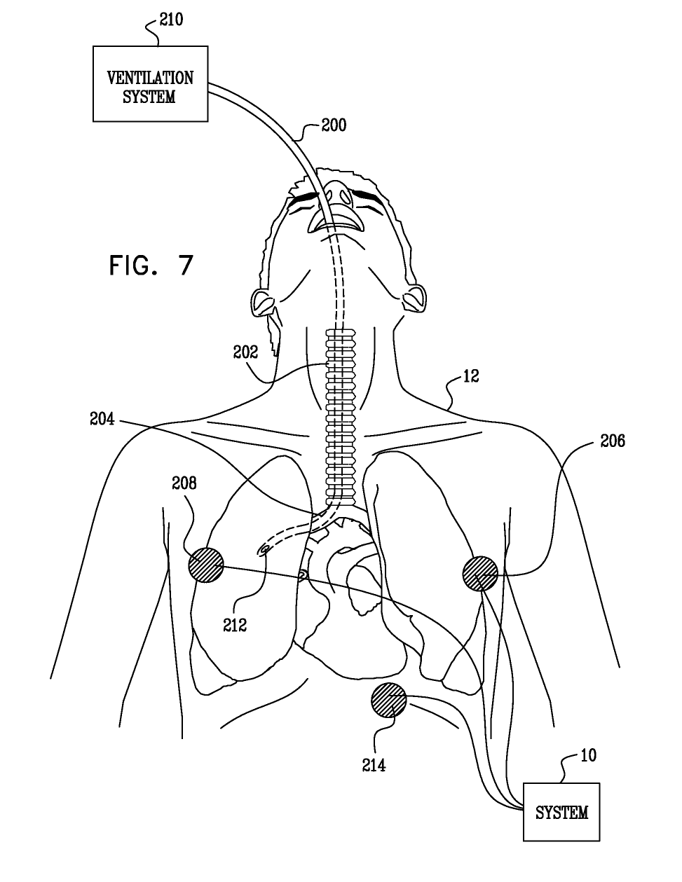

Reference is made to FIG. 7, which is a schematic illustration of system 10 applied to an intubated subject 12, in accordance with an embodiment of the present invention. In this embodiment, system 10 monitors subject 12 who is intubated for respiratory assistance. When performing such intubations, physicians need to ensure that an endotracheal tube 200 is placed in a trachea 202 above the carina and does not reach the right or left main bronchus 204. In most cases, the endotracheal tube should ventilate both lungs. In addition, it is considered important to maintain this proper positioning, and to ensure that the tube stays clear and unclogged. Furthermore, it is often desirable to identify whether the subject has a unilateral or segmental complication of the lung, such as pneumonia, atelectasis, or aspirations. In this embodiment, system 10 monitors intubated subject 12 with a single sensor 30 or a plurality of sensors 30. For example, sensors 30 may comprise two mechanical vibration sensors 206 and 208, which are positioned about 1 cm laterally to the nipples and measure the mechanical signal related to each lung's ventilation. Alternatively, the sensors are placed on the back of subject 12, one in the region of the right lung and one in the region of the left lung. The sensors detect a mechanical vibration and/or displacement signal, typically having a frequency of less than 20 Hz. When the subject is intubated appropriately, the system detects similar ventilation-related vibrations from the two detectors. If endotracheal tube 200 is malpositioned and located in one of the main bronchi, usually on the right side, the sensor or sensors on this side detect a significantly stronger signal and the system alerts the subject or clinician accordingly. Alternatively or additionally, the sensors are configured to detect an acoustic signal, and the system performs similar comparative processing. A larger number of sensors may be used to generate a more detailed identification of location of ventilation distribution in the lungs.

Additionally, for some applications, a visual image of the lungs and a color or intensity of the area of each lung is shown proportionally to the amplitude or other characteristic of the measured signal. For some applications, each lung is monitored by two to 10 sensors, for example three sensors covering different zones of each lung. The system displays an image conveying to the clinician the energy or frequency of the vibration signal detected in each zone. In addition, the system continuously calculates the ratios of the signals detected by the different sensors and alerts upon a significant change in these ratios. This embodiment provides the clinician with a convenient tool to monitor the effectiveness of ventilation as well as other lung characteristics.

In an embodiment of the present invention, sensors 30 are located on a plate in the bed (for example under the sheet), and the system detects the signal and/or displays the image when the subject lies above the sensor plate. Additionally, for some applications, the system builds a baseline of the amplitude of the ventilation signal (acoustic and/or mechanical), and, upon detecting a change in the amplitude of the overall signal or of one lung (i.e., in both lungs or one lung) greater than a threshold, the system generates an alert that an intervention may be required because of, for example, a clogged or malpositioned tube, obstruction of main or segmental bronchi by secretions, nosocomial pneumonia, effusions, pneumothorax, or other problems that result in impaired ventilation of the lungs. A clinician can then intervene and overcome this potentially life-threatening situation. Additionally, for some applications, the system analyzes the signal to identify the mechanical and acoustic signature of vomiting in order to identify and generate an alert when an intubated subject is vomiting, which is a potentially life-threatening situation. Additionally, for some applications, the system identifies aspirations and or changes in the vibration signature of each lung of an intubated subject and indicates a risk for the development of ventilator-associated pneumonia (VAP).

In an embodiment of the present invention, system 10 monitors the insertion procedure of endotracheal tube 200 by fixing mechanical vibration sensors on the back of the subject in the area of the lungs generally symmetrically in proximity to the right and left lungs (typically at least one sensor in the proximity of each lung). The healthcare worker inserts the tube into the trachea, such as not more than one cm into the trachea of a child, and two cm into the trachea of an adult. The healthcare worker then causes air to flow through the tube, and the system records the signal detected by the sensors. This initial signal serves as a calibration signal. The healthcare worker continues the insertion of the tracheal tube with ongoing air flow into the tube, and the system observes a pattern of the signal detected by the sensors. The tube is further inserted as long as the system does not detect a change in the pattern. Upon detecting a change in the pattern, the system alerts the healthcare worker that the tube may be malpositioned. The pattern analysis includes analyzing the level of symmetry between signals obtained from the one or more sensors positioned close to the right lung and the one or more sensors that are positioned close to the left lung. For example, the system may monitor whether the ratio of the amplitude of the signal measured from the proximity of each lung stays within set boundaries. The sensors may be consumable and replaced for different subjects. For some applications, the sensors comprise acoustic sensors. In addition, for some applications, a greater number of sensors is used and an image is presented to the clinician illustrating the data from each sensor.

In an embodiment of the present invention, system 10 monitors a ventilation system 210 providing air to endotracheal tube 200 in order to identify characteristic vibrations of the ventilation system. The system uses sensors 206 and 208 to identify the same characteristic vibrations in the lungs of the subject, and assesses the amplitude of these vibrations as an indication of the amount of air flowing into each lung from the ventilation system. For some applications, the system generates vibrations near the distal tip of tube 200 (or elsewhere in system 10), in order for the sensors to identify these vibrations. For example, the system may comprise a vibrating device 212 (e.g., a piezoelectric vibrating device) positioned in a vicinity of a distal end of the tube. Vibrating device 212 typically generates the vibrations in the acoustic frequency range or in a sub-acoustic frequency range of between about 1 and about 20 Hz. For some applications, ventilation system 210 or the vibrating device is configured to generate vibrations having a specific characteristic (e.g., a specific frequency or modulation pattern), and the system uses the sensors to identify this specific pattern.

In an embodiment of the present invention, the system comprises an additional sensor 214, which is placed on an external surface of the subject's body in a vicinity of the stomach. The system uses this additional sensor to monitor potential malpositioning of tube 200 into the esophagus. The system identifies that intubation tube 200 may have accidentally been inserted into the esophagus instead of the trachea if sensor 214 detects a substantial ventilation signal in the vicinity of the stomach, for example, a signal having a greater amplitude than the signal detected by sensors 206 and 208. The system alerts the clinician to correct the intubation error.

In an embodiment of the present invention, system 10 provides feedback to a clinician by generating an audio signal, so that the clinician does not have to look at the system and thus is able to concentrate his visual attention on the intubation procedure. The system typically provides feedback on both the balance between the two lungs and the amplitude of the signal. For example, the amplitude of the audio signal may represent the amplitude of the detected signal in both lungs, and the pitch of the audio signal may represent a level of difference in amplitude between the two lungs, and an error buzz may indicate detection of a substantial signal in the stomach. During an insertion procedure, the clinician learns to expect to hear a low-amplitude signal as the tube is inserted into the mouth, followed by a higher-amplitude signal when the tube enters the trachea (as the amplitude of the signal detected by the sensor increases when the tube enters the trachea). Subsequently, the clinician hears a change in pitch if he inserts the tube too far, such that the tube ventilates only one lung. Upon hearing such a change in pitch, the clinician pulls back the tube until the pitch returns to the level representing a relative balance between the lungs. Alternatively, instead of a change in pitch, the system generates another audio indication, such as a beeping sound having a rate of repetition proportional to the signal difference between the lungs.

In an embodiment of the present invention, the intubation monitoring system integrates the vibration sensors 206 and 208 (and optionally 214) and an additional sensor to validate the effectiveness of the ventilation system. For example, the additional sensor may comprise an end-tidal CO2 detector or a pulse oximeter.

In an embodiment of the present invention, system 10 generally continuously monitors the subject after completion of the intubation procedure, and provides a closed loop system with ventilation system 210. For example, if system 10 detects a degradation in the amplitude of the ventilation signal in the lungs, which may be caused by clogging of the tube, system 10 sends a signal to ventilation system 210 to automatically increase the flow output.

In an embodiment of the present invention, system 10 is configured to identify the onset of atelectasis in a lung or part of the lung by identifying a reduction in vibration or a change in the frequency distribution of the signal in the appropriate region covered by one or more of the sensors 206, 208, and/or 214.

In an embodiment of the present invention, sensors 206, 208, and/or 214 comprise piezoelectric ceramic sensors, acoustic sensors, accelerometers, strain gauges, and/or ultrasound detectors.

In an embodiment of the present invention, system 10 is configured to monitor a subject undergoing or having a tracheotomy, using techniques similar to those described above for monitoring intubation. System 10 is configured to indicate whether the subject is effectively ventilated. In some cases, subjects may acutely plug their tracheostomy. For some applications, system 10 provides a warning to a clinician upon such an event by detecting an acute change in respiratory pattern or body movement pattern.

In an embodiment of the present invention, system 10 is configured to classify the time during which a subject is monitored as wakeful periods, non-REM sleep periods, and REM sleep periods, based on analysis of respiration-related mechanical signal. The system typically bases the classification on movement detection and respiration irregularity/complexity analysis. The system typically categorizes movements combined with complex respiration activity as a wakeful period, complex respiration activity without movements as a REM sleep period, and non-complex respiration activity a non-REM sleep period.

Reference is made to FIG. 8, which is a flowchart schematically illustrating a method 250 for performing respiration complexity classification and sleep stage classification, in accordance with an embodiment of the present invention. In summary, among other things, the method extracts the following breathing regularity features from a signal: the standard deviation of respiration rate (BRSTD), standard deviation of respiration peak to peak amplitude (STDP2P), mean breath by breath correlation (MB2BC), and standard deviation of breath by breath correlation (STDB2BC), typically estimated within time windows of one minute. The method uses these features as inputs to a fusion algorithm which correlates detected movements and respiration complexity activity type, and classifies each time window as an awake period, a non-REM sleep period, or a REM sleep period. The sleep staging classification results are comparable to standard manual polysomnography (PSG) sleep stage classifications.

### Filtering

Method 250 begins with the receipt of a raw respiration signal 252 from one or more sensors 30. At a filtering step 254, system 10 performs band-pass, FIR, zero-phase digital filtering on raw respiration signal 252. For example, the cutoff frequencies of the filtering may be about 0.1 Hz and about 0.75 Hz. For some applications, zero-phase is obtained by first filtering the raw data in the forward direction, and subsequently reversing the filtered sequence and running the reversed filtered sequence through the filter again. The resulting sequence is zero-phased, such as described on pp. 311-312 of the above-mentioned book by Oppenheim et al.

### Feature Extraction for Movement and Noise Detection

For some applications, at a feature extraction step 256 system 10 uses a signal processing algorithm to perform feature extraction from raw respiration signal 252, in order to detect body movements and noise. The procedure operates on time windows of, for example, 30 seconds, with overlap of 29 seconds. The system estimates, from each time window, the variance (VAR), signal-to-noise ratio (SNR), and spectral-based breathing rate (SBR). In order to extract SNR and SBR, the system estimates the power spectrum of each time window using, for example, the Welch method, with FFT order of 1024 and overlap of 512. For some applications, the system estimates SNR using the following equation:

$$SNR = \frac{{\int }_{0.1}^{0.75} {P}_{xx} ( f ) \partial f}{{\int }_{0}^{Fs / 2} {P}_{xx} ( f ) \partial f} \cdot 100 \qquad ( Equation 8 )$$

wherein Pₓₓ denotes the power spectrum distribution function of the respiration signal, and Fs denotes the sampling rate in Hz.

For some applications, the system estimates SBR, which is measured in number of breaths per minute (bpm), using the following equation:

$$SBR = 60 \cdot {\int }_{0.1}^{0.75} {\tilde{P}}_{xx} ( f ) f \partial f \\ wherein {\tilde{P}}_{xx} ( f ) = \frac{{P}_{xx} ( f )}{{\int }_{0.1}^{0.75} {\tilde{P}}_{xx} ( f ) f \partial f} . \qquad ( Equation 9 )$$

### Peak and Minima Detection

For some applications, system 10 performs an algorithm for peak and minima detection in the respiration signal, at a peak and minima detection step 258. For some applications, the algorithm for peak detection comprises the following steps:
detect all maxima according to first derivative sign change using the filtered respiration signal generated at step 254; and
around each maximum point, open a time window with a duration adapted to the current SBR generated at step 256 (window size estimation is described hereinbelow), and verify whether the current maximum is a global maximum. If the tested maximum point is a local maximum, it is eliminated.
   For some applications, the system estimates the time window duration opened equally around each maximum point by finding the closest SBR point corresponding to an SNR greater than a threshold value, for example, about 50, within a time window having a certain duration, for example, about 5 minutes, and calculating the time window duration using the following equation:

TFD=60/SBR (Equation 10)

If the system finds that there is no SBR point corresponding to a SNR greater than a threshold value, for example, about 50, within a time window having a certain duration, for example, about 5 minutes, the system fixes the time window duration to a default value, for example, about 1.33 seconds.

The system identifies minima points by detecting the minimum between two consecutive maxima.

### Movement Detection

Reference is made to FIG. 9, which is a flowchart that schematically illustrates a method 270 for determining whether subject movement has occurred, in accordance with an embodiment of the present invention. At a movement detection step 260 of method 250 (FIG. 2), system 10 performs the method for movement detection shown in FIG. 9 for each time window, based on the VAR and SNR calculated for the window at feature extraction step 256, described hereinabove. For each time window having a duration of, for example, 30 seconds, for which the VAR and SNR features are extracted at step 256, system 10 uses method 270 to determine whether the window includes movement by the subject.

At an SNR threshold check step 272, the system compares the calculated SNR of the window to a threshold value, such as about 90. If the system finds that the SNR is less than the threshold, the system finds that no movement has occurred, at a no movement detection step 274. If, on the other hand, the system finds at check step 272 that the SNR is greater than or equal to the threshold, at a left- and rightward variance calculation step 276 the system calculates respective variances of a rightwards neighborhood and a leftwards neighborhood, which are sets of windows immediately following and proceeding the current window, respectively. The system calculates the rightward reference neighborhood variance (VRR) by accumulating, for example, five minutes of time windows, occurring after the tested time window, having SNRs greater than, for example, about 90, and calculating the mean variance of these time windows. The system uses the same technique for calculation of the leftward reference neighborhood variance (VLR), but for time windows occurring before the tested time window.

At a check step 278, system 10 calculates the ratios VAR/VRR and VAR/VLR for the window, using the VAR for the window calculated at feature extraction step 256 of method 250, and the VRR and VRL calculated at step 276 of method 270. The ratios are the ratios between the variance of the tested window to the mean variances of the right and left neighborhoods, respectively. If the greater of these two ratios is greater than a threshold (denoted “ENERGYTHRESH” in FIG. 9), the system finds that movement has occurred, at a movement detection step 280. Otherwise, the system finds that no movement has occurred, at movement detection step 274.

### Noise Detection

Reference is again made to FIG. 8. In an embodiment of the present invention, at a noise detection step 282, system 10 performs an algorithm for detecting noise, i.e., a portion of the signal in which no respiration signal is measured, based on the SNR calculated at feature extraction step 256, described hereinabove. For each time window of, for example, 30 seconds, for which the SNR feature is extracted, the system determines that the time window includes a noise period if its corresponding SNR is less than a threshold value, for example, about 60.

### Respiration Regularity Feature Extraction

Reference is still made to FIG. 8. In an embodiment of the present invention, at a regularity feature extraction step 284, system 10 performs an algorithm for the extraction of breathing regularity features based on a Bayesian classifier. For some applications, the system extracts the features from time windows having a duration of, for example, 60 seconds, with an overlap of, for example, 50 seconds. The features comprise one or more of the following: (1) standard deviation of instantaneous breathing rate (BRSTD), (2) standard deviation of peak-to-peak amplitude of the respiration signal (STDP2P), (3) mean value of breath-to-breath correlation (MB2BC), and/or (4) standard deviation of breath-to-breath correlation (STDB2BC).

For some applications, the system estimates breathing rate using the following equations:

$$\begin{matrix}{BR}^{( ma x ) } ( {t}_{k}^{ma x} ) = \frac{60}{{t}_{k}^{ma x} - {t}_{k - 1}^{ma x}} & ( Equation 11 ) \\ {BR}^{( m i n )} ( {t}_{k}^{m i n} ) = \frac{60}{{t}_{k }^{m i n} - {t}_{k - 1}^{m i n}} & ( Equation 12 )\end{matrix}$$

wherein tₖ^{max} and tₖ^{min} are maximum and minimum points in the respiration related motion signal, respectively. It is noted that the breathing rate is estimated twice, once according to maxima points and a second time according to minima points. Within each time window of, for example, 60 seconds, the system selects the minimal standard deviation of breathing rate.

For some applications, the system calculates peak-to-peak amplitude using the following equation:

P2P(tₖ^{max})=Amp(tₖ^{max})−Amp(tₖ^{min}) (Equation 13)

For some applications, the system estimates breath-by-breath correlation using the following equation:

P2P(tₖ^{max})=max corr[Amp(tₖ₋₁^{min}, . . . , tₖ^{min}),Amp(tₖ^{min}, . . . ,tₖ₊₁^{min})] (Equation 14)

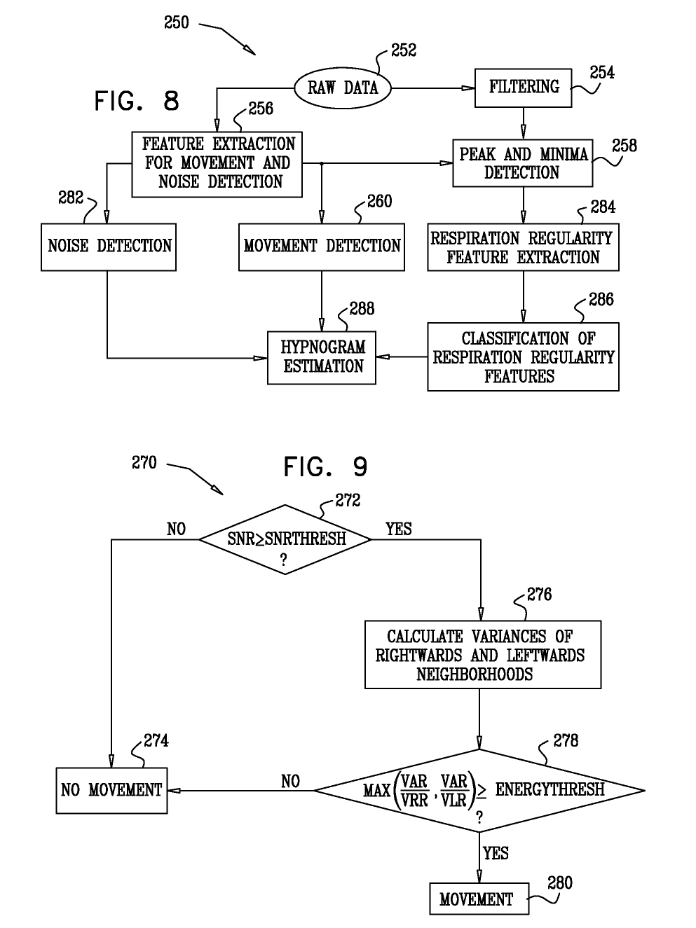

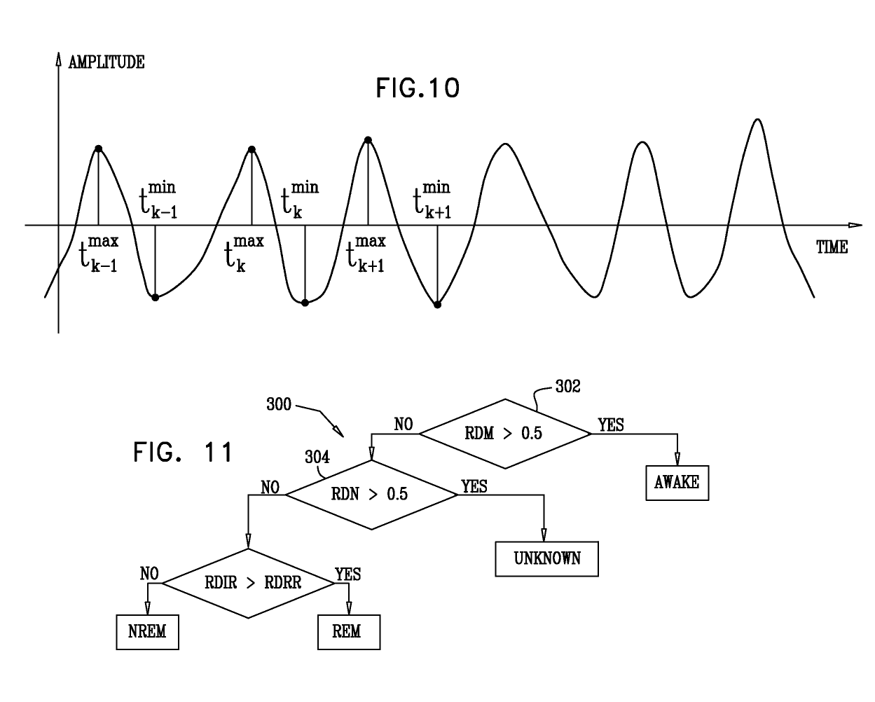

FIG. 10 is a schematic illustration of an exemplary respiration signal and the maxima and minima points used for feature extraction, in accordance with an embodiment of the present invention.

### Classification of Respiration Regularity Features

In an embodiment of the present invention, at a classification step 286, system 10 performs algorithms for classification of vectors of the clinical parameters defined hereinabove with reference to step 284 of method 250 of FIG. 8. In an embodiment, the vector is a four dimensional feature vector, corresponding to a time window having a duration of, for example, 60 seconds. The system classifies each feature vector into one of the following three classes: (1) regular breathing, (2) irregular breathing, or (3) highly irregular breathing. For some applications, the system models a probability density function of the observations using the following equation:

$$f ( {x}_{t} ; {θ}^{( x )} ) = {\sum }_{k = 1}^{3} {v}_{k} {f}_{k} ( {x}_{t} ; {θ}_{k}^{( x )} ) , t = 1 , … , T \qquad ( Equation 15 )$$

wherein xₜ, θ^{(x)}={θₖ^{(x)}}ₖ₌^{K}, and vₖ denote an observation (feature) vector at time instance t, the distribution parameters of the observations, and the a priori probability of the k^{th} class, respectively. The distribution parameters of an observation vector, given the k^{th} class, is denoted by The probability density function (PDF) of each class is modeled via the Gaussian mixture model (GMM), for example using techniques described in the above-mentioned article by Li et al. For example, the following equation may be used:

$${f}_{k} ( {x}_{t} ; {θ}_{k}^{( x )} ) = {\sum }_{m = 1}^{{M}_{k}} {w}_{m}^{( k )} N ( {x}_{t} ; {μ}_{m}^{( k )} , {R}_{m}^{( k )} ) , \qquad ( Equation 16 )$$

wherein Mₖ, {wₘ^{(k)}}ₘ₌₁^{M}^{ₖ}, and N (•;•,•) denote the number of Gaussians in the k^{th} class, the Gaussian weights, and the multivariate normal PDF, respectively. The mean vector and covariance matrix of the PDF of the m^{th} Gaussian of the k^{th} class are denoted by μₘ^{(k)} and Rₘ^{(k)}, respectively.

For some applications, the system performs classification using the following equation:

$$c ( t ) = arg {max}_{k} [ f ( {θ}_{k}^{( x )} \vert {x}_{t} ) ] = arg {max}_{k} [ \frac{P ( {θ}_{k}^{( x )} ) f ( {x}_{t} ; {θ}_{k}^{( x )} )}{f ( {x}_{t} ; {θ}^{( x )} )} ] = arg {max}_{k} [ {v}_{k} f ( {x}_{t} ; {θ}_{k}^{( x )} ) ] \qquad ( Equation 17 )$$

wherein the classification decision at time instance t is denoted by c (t). Parameter estimation of the classifier is described in the section hereinbelow entitled, “Classifier design and parameter estimation.”

### Hypnogram Estimation

Reference is made to FIG. 11, which is a flowchart schematically illustrating a method 300 for classifying sleep stages, in accordance with an embodiment of the present invention. In this embodiment, at a hypnogram estimation step 288 of method 250 (FIG. 8), system 10 performs an algorithm for classification of sleep stages, typically including the following: awake, non-REM sleep (NREM), and REM sleep (REM). The system performs sleep staging on non-overlapping time windows of, for example, one minute. In an embodiment, the system calculates one or more of the following parameters within each time window: (1) relative duration of movement activity (RDM), (2) relative duration of noise (RDN), (3) relative duration of regular respiration periods (RDRR), (4) relative duration of irregular respiration (RDIR), and/or (5) relative duration of highly irregular respiration (RDHIR). The system applies classification method 300 of FIG. 11 to these calculated parameters to each time window. At each step of method 300, the system performs a comparison. For example, at a first check step 302 of the method, the system compares the calculated RDM to a constant, such as 0.5. If the RDM is greater than the constant, the system determines that the subject is awake. Otherwise, the system proceeds to a second check step 304 of the method (for which the exemplary value of 0.5 is shown for the constant in the comparison of this step).

The system typically smoothes the classification results in non-overlapping time windows having durations of, for example, 2.5 minutes. For each time window, the system identifies which sleep stage has the maximum duration, and classifies the sleep as characterized by this stage.

### Classifier Design and Parameter Estimation

In an embodiment of the present invention, system 10 includes algorithms for estimation of the classifier parameters used in Equations 15 and 16 hereinabove, namely {v_{k}}ₖ₌₁³, {wₘ^{(k)}}_{k,m}^{K,M}^{ₖ}, {μₘ^{(k)}}_{k,m}^{K,M}^{ₖ}. In order to estimate the distribution parameters of each class, the system uses features corresponding to awake, NREM, or REM periods scored on a learning set of subjects simultaneously monitored by a polysomnography (PSG) test. Segments greater than 5 minutes are collected into C₁, C₂, and C₃ clusters, respectively. Features corresponding to noise or movement periods are typically discarded.

For some applications, the system estimates the a priori probability of each class, denoted by {vₖ}ₖ₌₁³, using the following equation:

$${v}_{k} = \frac{N ( {C}_{k} )}{{\sum }_{k = 1}^{3} N ( {C}_{k} )} \qquad ( Equation 18 )$$

wherein N (Cₖ) denotes the number of feature vectors in the k^{th} cluster.

The system estimates the distribution parameters of each class θₖ^{(x)}={wₘ^{(k)},μₘ^{(k)},Rₘ^{(k)}}ₘ₌₁^{M}^{ₖ}, such as by using the EM algorithm suggested in the above-mentioned article by Dempster et al. for GMM parameter estimation (see the above-mentioned article by Bilmes). The optimal number of Gaussians is determined using the Bayesian information criterion (BIC) (see the above-mentioned article by Schwarz).

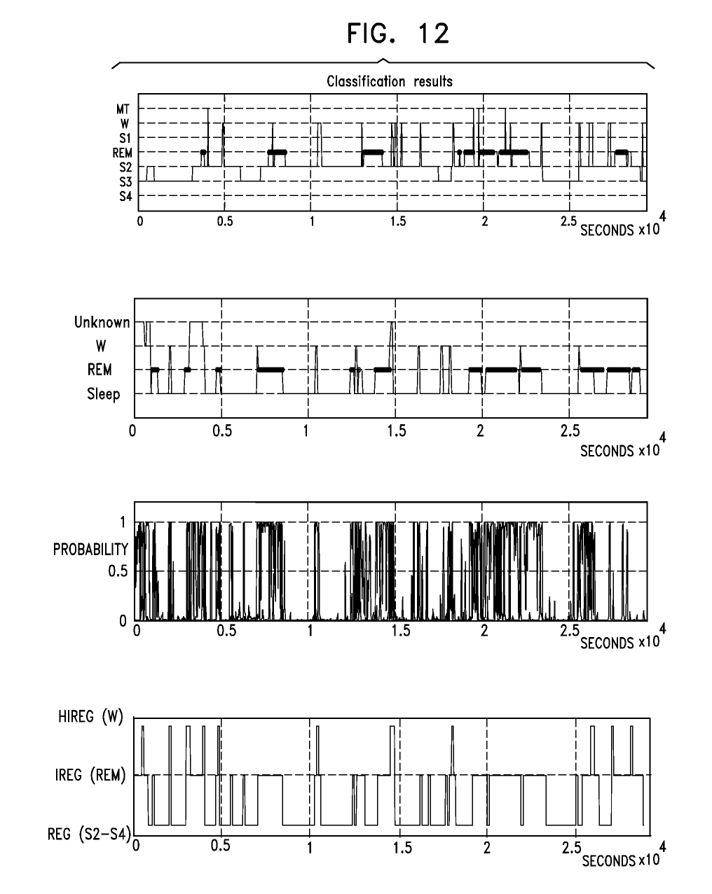

Reference is made to FIG. 12, which includes graphs showing experimental results obtained in accordance with an embodiment of the present invention. An experiment was performed comparing the results of classification method 300 of FIG. 11 to standard sleep lab analysis results. The top graph in FIG. 12 shows representative results of manual scoring for a subject using standard sleep lab equipment, and the second graph in FIG. 12 shows the results obtained for the same subject for the same period using classification method 300 of FIG. 11. As can be seen, there was a high correlation between the classification performed using techniques of the present invention and those obtained using standard sleep lap equipment.

The third graph in FIG. 12 depicts the a-posteriori probability of each breathing pattern class (highly irregular respiration, irregular respiration, and regular respiration) as a function of time. Each time point corresponds to a feature vector, which corresponds to a respiration time frame of 60 seconds. The fourth graph in FIG. 12 depicts the classification results of each feature vector into one of the three breathing pattern classes, described above, as a function of time, using Equation 17 above. Each time point corresponds to a feature vector, which corresponds to a respiration time frame of 60 seconds.

In an embodiment of the present invention, system 10 uses changes in length and periodicity of the different sleep stages as additional clinical parameters to predicting an impending onset of a chronic condition, such as an asthma attack, congestive heart failure deterioration, cystic fibrosis-related deterioration, diabetes hypoglycemia, or epilepsy deterioration. For some applications, the system uses the method 300 described hereinabove with reference to FIG. 11 to identify the time and duration of deep sleep periods. For some applications, system 10 is configured to identify the time, duration, and periodicity of REM sleep segments. The system uses these parameters as additional clinical parameters for which the system creates a baseline, identifies changes vs. baseline, and uses these changes to predict and/or monitor a clinical condition. For example, a change in the baseline periodicity of REM sleep for subject 12 may indicate the onset of an asthma attack or pulmonary edema.

In an embodiment of the present invention, during sleep, the system identifies sleep stage using techniques described hereinabove with reference to FIG. 11. For each identified sleep stage, the system calculates the average respiration rate, heart rate, and other clinical parameters. The system compares these calculated parameters to baseline values of these parameters defined for the particular subject for each identified sleep stage, in order to identify the onset or progress of a clinical episode.

In an embodiment of the present invention, system 10 performs an analysis of the parameters described hereinabove with reference to regularity feature extraction step 284, namely BRSTD, STDP2P, MB2BC, and STDB2BC, in combination with the algorithms for monitoring and predicting the deterioration of asthma, COPD, CHF, and/or other clinical conditions, by creating a baseline of these parameters and determining the change in these parameters compared to baseline. In addition, for some applications, system 10 integrates these parameters into the clinical score calculated for subject 12, as described hereinabove.

In an embodiment of the present invention, system 10 is used to monitor subjects with tuberculosis in order to identify and alert upon a change in the condition of subject 12. Increases in respiration rate, heart rate, cough or restlessness in sleep may indicate that the subject's overall condition is deteriorating.

Reference is again made to FIG. 2. In an embodiment of the present invention, pattern analysis module 16 extracts breathing rate from a continuous heart rate signal using amplitude demodulation, e.g., using standard AM demodulation techniques. This is possible because respiration-related chest wall movement induces mechanical modulation of the heartbeat signal.

In an embodiment of the present invention, pattern analysis module 16 uses an amplitude- and/or frequency-demodulated heart rate signal to confirm adequate capture of the breathing and heart rate signals, by comparing the breathing rate signal with the demodulated sinus-arrhythmia pattern extracted from the heart-rate signal. For some applications, the sinus-arrhythmia pattern is frequency-demodulated by taking a series of time differences between successive heart beats, providing a non-biased estimate of the ongoing breathing pattern. Alternatively or additionally, the heart beat is amplitude-demodulated using high-pass filtering, full-wave rectification, and low-pass filtering. The system monitors the level of modulation of the heart rate by the respiration rate, i.e., the change in the frequency and amplitude of the heart beat related signal, and uses this level of modulation as an indication of the subject's condition. For some applications, the system integrates the level of modulation into the subject's clinical score, as described hereinabove.

In an embodiment of the present invention, system 10 is used to monitor subjects with high cord spinal injury, in order to provide an early indication of deterioration (e.g., fever) detected responsively to a change in monitored clinical parameters, such as respiration rate, heart rate, cough count, and sleep quality.

In an embodiment of the present invention, system 10 is used as a tool to provide an indication that a subject is at risk of dehydration. Dehydration is often characterized by a change in respiratory rate and heart rate.

In an embodiment of the present invention, system 10 is configured to identify large body movement of subject 12. Large body movements are defined as having an amplitude that is substantially greater (e.g., at least 5 times greater) than that of respiration-related body movement, and/or having frequency components that are higher than those of respiratory motion (e.g., frequencies greater than about 1 Hz). For some applications, the system extracts relative and absolute movement time and amplitude parameters from the mechanical signal. The signal pattern prior to movement corresponds either to regular breath (when the subject is in the bed) or to system noise (the subject is entering to the bed). The signal pattern during large body movement is characterized by high amplitude in the range of 5 to 100 times greater than regular breath amplitudes, and by rapid signal change from maximum positive value to minimum negative value. The initial large body movement phase that consists of the transition from the pattern corresponding to regular breath or system noise to the movement pattern typically has a duration of about 0.5 seconds. The typical duration of the large body movement event ranges between 10 and 20 seconds. The dynamics of the initial phase are characterized by change of signal to maximum amplitude during one second. During the initial phase of the large body movement, increase in amplitude is typically in the range of 10 to 100 times greater than the maximum value corresponding to regular breath pattern.

In an embodiment of the present invention, system 10 identifies the start of the large body movement event by detecting the initial movement phase, and the end of the movement event when the movement phase concludes. For some applications, the system performs real-time signal analysis by evaluating sliding overlapping windows, and identifying the initial movement phase as occurring during a window characterized by at least one of the following ratios, or, for some applications, by both of the following ratios:
a signal-to-noise ratio (SNR) that is less than a threshold value; and
a ratio of the signal standard deviation (STD) during the window to the signal STD during a window characterized by a typically respiratory signal (e.g., the most recent window in which a respiratory signal was detected), which is greater than a threshold value.
   To calculate the SNR, the system typically calculates the power spectrum, and sets the SNR equal to the ratio of: (a) the energy in a specific frequency interval in the respiratory range (e.g., between about 0.1 and about 1 Hz) to (b) the energy of the noise in the entire spectrum excluding the respiratory range. The frequency interval is similar to the range of respiration rates detected by the system. The system typically specifies a window size such that each window includes at least one respiratory cycle (e.g., 5 seconds if the breathing rate is 12 breaths/minute). For some applications, the system adaptively sets the window size, while for other applications the system fixes the window size according to the lowest allowed respiratory rate.

Alternatively, the system performs the detection of the movement initial phase of the large body movement by dividing the time window into small windows having a duration of between about 0.5 and about 0.75 seconds (with or without overlapping). For each window, the system calculates a set of parameters based on the signal variance within the window. For some applications, the system sets the variance equal to the sum of absolute values of pairs of sequential samples differences normalized by the square root of the number of samples in the window. The system compares the variance parameter to a threshold, and if the variance parameter is greater than the threshold, the system identifies the window including a large body movement.

In an embodiment, system 10 is configured to detect bed entry and/or exit by subject 12. The system identifies bed entry upon detecting large body movement followed by a signal indicative of continuous motion (e.g., related to respiration or heartbeat), and bed exit upon detecting large body movement followed by a lack of motion signal. For some applications, sensor 30 comprises a single semi-rigid plate, and, coupled thereto, a vibration sensor and two strain gauges that are configured to detect the weight the subject's body applies to sensor 30.

In an embodiment, system 10 is used to monitor subjects during transport in a stretcher. The sensor is implanted within the fabric of the stretcher and continuously monitors the subject during transport. System 10 generates an alert upon detecting an acute change in subject condition without requiring any activation by the clinician or any compliance by the subject.

In an embodiment of the present invention, system 10 is configured to identify a change in the condition of at least one subject in a hospital, such as in a surgical or medical ward, such as by using techniques described in U.S. patent application Ser. No. 11/782,750, which is assigned to the assignee of the present application and incorporated herein by reference. The change typically includes a deterioration that requires rapid intervention. System 10 typically identifies the change without contacting or viewing the subject or clothes the subject is wearing, without limiting the mobility of the subject, and without requiring any effort by the nursing staff or other healthcare workers. For example, upon detecting a decrease in the subject's respiration rate to below eight breaths per minute, which may be a sign of respiratory depression, the system may generate an alert to a nurse. For some applications, the system is configured to predict an onset of a clinical episode, and to generate an alert.

For some applications, system 10 monitors the subject in the hospital automatically upon entry of the subject into a subject site such as a bed. Typically, system 10 does not require activation by a nurse or other healthcare worker, and no compliance by the subject is required other than to be in bed. Typically, motion sensor 30 is contactless (i.e., does not contact the subject or clothes the subject is wearing), and operates substantially continuously. When the subject enters the bed, the sensor detects the vibrations or other movements generated by the subject and initiates monitoring.

Alternatively or additionally, the system uses the technique described hereinabove for detecting bed entry. The system alerts clinicians upon any change that may require intervention. For example, the system may send an alert to a nurse, a member of a rapid response team, or other healthcare worker, such as wirelessly, e.g., to a wireless communication device, such as a pager, or using another call system in the hospital. For some applications, upon receiving the message, the wireless communication device sounds an audible alert, e.g., including an automatically generated voice message that includes the subject's name or number, room number, and/or alert type. This enables a clinician to act upon the alert and/or assess the situation without having to handle the pager (which is useful in situation where the clinician's hands are busy).

For some applications, when the subject enters the bed, system 10 initially uses a preset threshold for alerts. Over a period of time, e.g., one hour, the system establishes a reference baseline, e.g., the average respiration rate over that time period. Once the baseline has been established, upon identifying a change (e.g., a rapid change) in a clinical parameter versus the baseline, the system alerts a clinician. For example, the system may generate an alert upon detecting a change of 35% in a clinical parameter rate within a 15 minute period.

For some applications, the system makes a decision whether to generate an alert responsively to at least one clinical parameter selected from the group consisting of: a current value of the clinical parameter, a change in the clinical parameter versus baseline, and a rate of change of the clinical parameter over a relatively brief period of time, such as over a period of time having a duration of between about 2 and about 180 minutes, e.g., between about 10 and about 20 minutes. For some applications, the system uses a score which combines two or more of these parameters. For example, the score may include a weighted average of two or more of the parameters, e.g.:

Score=K*Param+J*DeltaParam+L*DeltaParamRate (Equation 19)

wherein K, J, and L are coefficients (e.g., equal to 1, 0.2, and 0.4, respectively); Param is the current value of the clinical parameter, for example respiration rate or heart rate; DeltaParam is the difference (e.g., expressed as a percentage) of the parameter versus the subject's baseline; and DeltaParamRate is the change in percent of the parameter between the current time and that in a previous time period, for example between about 10 and about 20 minutes earlier, e.g., about 15 minutes earlier. Typically, Param has a unit of measurement, e.g., breaths per minute, or heartbeats per minute, while DeltaParam and DeltaParamRate do not have units. For some applications, Param is normalized, such as by dividing the measured value by the baseline value and multiplying by a constant, e.g., 100. For example, the upper and lower thresholds for Score (if Param is normalized) may be set to 65 and 135, respectively, for monitoring respiration rate. If Score falls outside the range between the thresholds, the system generates an alert. In an embodiment, sensor 10 is implemented inside the mattress of the bed, thereby adding no visible extra parts to the bed.

In some embodiments of the present invention, including the embodiment described immediately above, it is generally desirable to minimize alarms, especially alarms that activate the nurse call system and are heard throughout the ward in a hospital. In an embodiment, upon identifying cause for alert, system 10 first activates a local alarm in the subject's room for a brief period of time, e.g., 30 seconds. User interface 24 of system 30 comprises a deactivation control, such as a button, that allows a clinician who is in the room to deactivate the alarm, thereby preventing the activation of an alarm throughout the entire hospital ward. After the brief period of time, if the local alarm was not deactivated by a clinician, the system generates the general alert.

For some applications, sensor 30 is installed in a subject site such as a chair near the subject's bed.

For some applications, the system deletes the baseline upon detecting that the bed is empty for a certain period of time, e.g., one hour, which may indicate that the subject has left the bed and a new subject has entered the bed.

For some applications, system 10 comprises user interface 24, which is configured to accept input from a clinician of information regarding: (a) the assigning of a new subject to the bed, (b) threshold levels appropriate for a particular subject, and/or (c) other information regarding a particular subject, such as the health condition of the subject, or known parameters for the risk of pressure sores (e.g., bed sores) or the risk of the subject falling out of the bed.

In an embodiment of the present invention, system 10 identifies Cheyne-Stokes respiration (CSR) and activates the nurse call system upon detecting that the CSR has a higher frequency than a threshold frequency.

In an embodiment of the present invention, system 10 comprises one or more of the following sensors: a urine output sensor, a temperature sensor (wired or wireless), and a blood pressure sensor.

In an embodiment of the present invention, system 10 is used to monitor subject 12 following physical exercise in order to identify the pattern and time of return of the heart rate and respiration rates to normal. For some applications, sensor 30 is installed in a couch. Subject 12 sits on the couch upon completing the exercise, and the system monitors and logs his parameters until they stabilize or for as long as the subject remains on the couch.

In an embodiment of the present invention, system 10 detects pulse and respiratory movement. These signals are fed into an imaging system, such as a CT or an MRI imaging system, as a gating signal, in order to improve image quality and prevent respiration/heart beat motion artifacts. For some applications, a contactless sensor is integrated into the bed of the imaging system.

In an embodiment of the present invention, sensor 30 is installed in a chair at the subject's bedside. For some applications, the system deletes the baseline upon detecting that the bed and/or chair is empty for more than one hour, which may signify that the subject has left the bed, and a different subject may enter the bed.

In an embodiment of the present invention, system 10 is configured to identify early warning signs of pulmonary embolism. These signs include a quick change in respiratory rate vs. baseline (for example, change over a duration of between about 1 and about 60 minutes, typically about 10 minutes), restlessness, and, in some cases, coughing. For some applications, upon detection of one or more of the above signs in a subject at risk for deep vein thrombosis (DVT), system 10 generates an alert for a clinician that a risk of pulmonary embolism has been identified. The alert enables the clinician to intervene and prevent the serious risks of complications.

In order to reduce the risk of DVT and pulmonary embolism, sequential compression devices (SCDs) are often used to improve venous return. In an embodiment of the present invention, system 10 is used in conjunction with an SCD, such as in a home or hospital environment, to monitor subjects who are at risk of pulmonary embolism and to provide early warning for the onset of pulmonary embolism. For some applications, system 10 also identifies characteristic vibration generated by the SCD and logs the time and lengths of the use of the SCD, and, alternatively or additionally, generates an alert upon finding that the SCD has not been used for a period of time longer than a threshold value, typically input into the system by a clinician. For some applications, sensor 30 is embedded within the SCD.

In an embodiment of the present invention, system 10 is used to monitor subjects and generate an alert upon detecting a deterioration. For some applications, pattern analysis module 16 is fed information about patterns of specific types of deteriorations, such as pulmonary embolism, hypoglycemia, and alcohol withdrawal. The clinician selects for which types of conditions the subject is at risk, and the system looks up a set of parameters appropriate for the selected conditions, and generates an alert for these conditions. For example, tachycardia, palpitations, tremor, agitation in sleep, and seizures are symptoms for alcohol withdrawal; tremor and tachycardia are symptoms for hyperglycemia; and tachypnea, tachycardia, and coughing are symptoms for pulmonary embolism. The system checks for the combinations that fit the conditions that the clinician has selected, and generates an alert upon identifying any of these combinations. This technique provides effective early warning for the clinician, while reducing false alarms for events that are highly unlikely for a specific subject (e.g., hypoglycemia is unlikely for a subject who does not have diabetes, and pulmonary embolism is unlikely for a subject with no known risk for DVT).

It is recommended that most hospitalized subjects avoid staying in bed continuously for extended periods of times. In an embodiment of the present invention, system 10 measures how long the subject stays in bed continuously. The system logs the data and optionally generates an alert for a clinician if the length of time exceeds a threshold value, e.g., set by the clinician.

In an embodiment of the present invention, sensor 30 is installed within a bed mattress as an integral part of the mattress.

Reference is again made to FIG. 2. In an embodiment of the present invention, system 10 monitors subjects in a hospital with a contactless mechanical sensor (sensor 30) and acoustic sensor 82. The system identifies audio signals that correlate with the motion signal as belonging to the subject. The system identifies snoring and wheezing, for example, and generates an alert for a clinician. For some applications, the system identifies talking by the subject by detecting a combination of vibration signal and audio signal. While the subject is talking, the system configures the heart rate and respiration rate detection algorithms so as not to mistake the talking-related body motion with respiration or heart rate data.

In an embodiment of the present invention, mechanical sensor 30 comprises a piezoelectric ceramic sensor that is coupled to a semi-rigid but flexible plate, comprising, for example, polymethyl methacrylate (PMMA), acrylonitrile butadiene styrene (ABS), or polycarbonate, and having a thickness of between about 1 and 5 mm, e.g., about 2 mm and dimensions of about 20 cm by about 25 cm. As used in the present application, including in the claims, “semi-rigid” means partially but not fully rigid, such that the plate generally maintains its shape when not subjected to force, and is able to bend somewhat without breaking when subjected to a moderate force, such as pressure applied by a mattress. The plate serves effectively as an antenna that collects the vibrations from under the mattress, mattress pad, or mattress cover. The sensor is coupled to the plate and detects the vibration of the plate. The plate also protects the sensor from breaking (the sensor generally breaks if bent more than 5 degrees).

In an embodiment of the present invention, a sensor assembly is provided that comprises a plate and at least two sensors coupled to the plate. The use of at least two sensors generally provides for improved signal detection, while maintaining the convenience of a single plate. For some applications, one of the sensors is placed under the area of the subject's legs and another of the sensors is placed under the area of the abdomen, such as to provide a plurality of signals from which the signal processing unit selects to calculate the clinical parameters (or to combine the various signals).

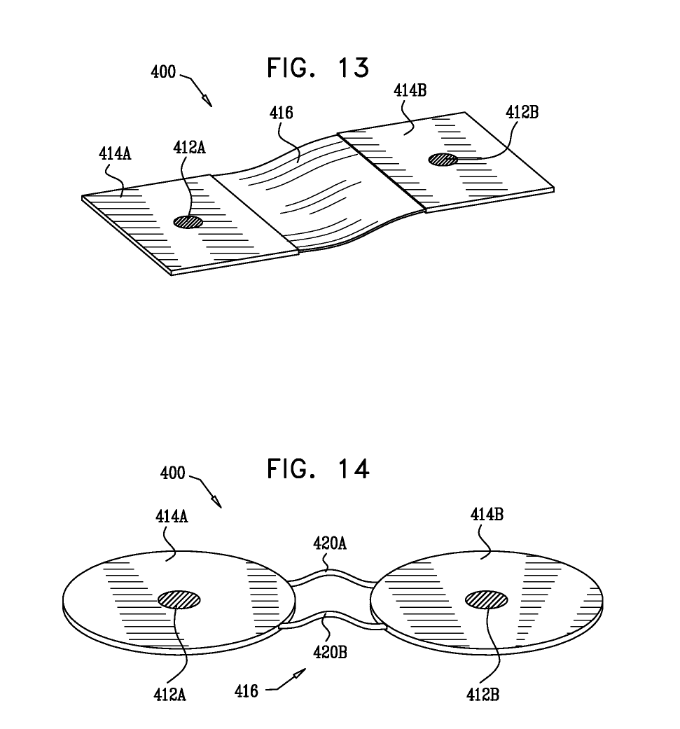

Reference is made to FIG. 13, which is a schematic illustration of a sensor assembly 400, in accordance with an embodiment of the present invention. Many beds include an option to adjust the angle of the upper body area of the bed. Thus, if a multi-sensor, semi-rigid plate were to be placed on the bed with one sensor in the area of the legs and one sensor in the area of the abdomen, inclining the upper body area of the bed may cause the plate to break. For some applications, in order to prevent such breakage, a sensor assembly 400 comprises at least two semi-rigid plates 414A and 414B, at least two sensors 412A and 412B coupled to respective plates, and a flexible connecting element 416 that couples semi-rigid plates 414A and 414B to one another. For example, the flexible connecting element may comprise bendable rubber. The sensory assembly is placed under the mattress or mattress cover such that flexible connecting element 416 is located in the area of the bed where the angle may change and the two semi-rigid plates are placed in the areas of the legs and the abdomen, respectively. This design provides the clinician the convenience of a single, potentially disposable, sensor assembly, while allowing the subject to change the angle of the bed without breaking the sensor assembly. For some applications, each of semi-rigid plates 414A and 414B has a thickness of between about 1 and about 5 mm, such as about 2.5 mm, a width of between about 15 and about 30 cm, such as about 20 cm, and a length of between about 20 and about 40 cm, such as about 30 cm, and flexible connecting element 416 has a thickness of between about 0.2 and about 3 mm, such as about 1 mm, a width of between about 12 and about 30 cm, such as about 20 cm, and a length of between about 1 and about 50 cm, such as about 20 cm.

FIG. 14 shows a schematic illustration of another configuration of sensory assembly 400, in accordance with an embodiment of the present invention. In this configuration, flexible connecting element 416 comprises one or more elastic bands 420A and 420B.

For some applications, the width of the plate(s) is configured to cover the entire width of the bed (e.g., 90 cm for a typical hospital bed), such that the plate collects vibrations generated by the body even if the subject is lying at the edge of the bed.

In an embodiment of the present invention, sensor 30 comprises a first piezoelectric sensor coupled to a semi-rigid plate, as described hereinabove, which is used with an electric circuit that is configured to switch between two modes. In a first of the modes, the system reads the signal from the sensor as described hereinabove. In a second of the modes, the system drives an electrical voltage/current into the first sensor with a frequency that is typical of the signal that is generally read by the first sensor from a biological signal source, e.g., between about 0.05 Hz and about 20 Hz. This signal causes the semi-rigid plate and the piezoelectric sensor to vibrate. The sensor assembly further comprises a second sensor coupled to the plate, which second sensor is configured to detect the vibration generated by the first sensor. The amplitude and shape of the detected vibration signal is used to validate that the first and second sensors are functional. For example, if the first sensor or the plate is broken, the second sensor detects a lower amplitude signal and/or a deformed signal. For some applications, the system drives the first sensor to generate a signal that sweeps a frequency range in order to verify that the first sensor is fully functional at all or a plurality of relevant frequencies. For some applications, the sensor plate is initially calibrated and a baseline frequency response is measured using these techniques and logged in the system. The system periodically performs this test in order to detect whether there has been in change in the frequency response. If the system detects a change larger than a set threshold, the system generates an alert for the user, a healthcare worker, and/or a vendor of the system. For some applications in which the system uses two sensors for sensing, the system uses each of the sensors to test the other sensor.

In an embodiment of the present invention, the test procedure is implemented using only a single sensor coupled to the plate. The electric circuit drives the sensor to generate vibration of the sensor and plate. The electric circuit rapidly switches from vibrating mode to detection mode while the plate is still vibrating (e.g., the switching is performed in less than 0.01 seconds, while the vibration continues for at least 0.3 seconds). The circuit detects the vibration of the plate, as described above, and compares the detected vibration to baseline.

In an embodiment of the present invention, sensor 30, e.g., the sensor plate described hereinabove, is placed within or below a pillow. The sensor uses wireless communication to transmit the sensed signal to the processing unit. The pillow thus serves as a wireless sensing element that may accompany the subject as he moves from one bed to another, from the bed to a chair or a couch, or from one side of the bed to another.

In an embodiment of the present invention, system 10 monitors subjects using a plurality of sensors 30. The sensors are configured to be cascaded one to the next through a wired or wireless communication interface. The system collects all data from the sensors into the processing unit. The processing unit selects the sensor with the best data according to criteria based on signal-to-noise ratio, or combines the data through cross correlation and other appropriate signal processing algorithms.

A subject who is at risk of pressure ulcers is often placed on an alternating pressure mattress that is intended to vary the points on the subject's body that are in contact with the bed. In an embodiment of the present invention, each time the pressure mattress is activated to change position, system 10 detects the mechanical signal (i.e., the vibration) generated by the pressure mattress and incorporates this vibration into the detection algorithm so as not to mistakenly identify this vibration as a respiration or heart rate signal. Alternatively, system 10 learns a characteristic vibration signature of the pressure mattress system and pattern analysis module 16 identifies the signal each time it occurs in order to disregard it.

In an embodiment of the present invention, system 10 calculates a confidence level for each clinical parameter detected. The confidence value is calculated, for example, for the respiration rate by calculating the signal-to-noise ratio in the frequency domain of the peak related to the respiration rate to the baseline noise level of the frequency spectrum. The system uses the confidence level to minimize false alarms. Thus, for example, if the respiration rate crosses a threshold set for an alarm, but the confidence level is not sufficiently high, the system may wait for an additional reading (e.g., 30 seconds later) before activating the alarm.

In an embodiment of the present invention, system 10 identifies change of posture of a subject using exactly one sensor by identifying the change in the amplitude of the signal.

In an embodiment of the present invention, system 10 is used to monitor animals. In an embodiment of the present invention, vibration 30 and acoustic sensor 82 are placed within an oxygen therapy chamber in which the respiration of the animal is monitored.

In an embodiment of the present invention, system 10 identifies time periods without large body motion (quiet segments) and time periods with large body motions. The system logs the length of each quiet segment, and analyzes the distribution of the time lengths of the quiet segments over a period of time between about 15 minutes and about one day, such as about six hours. In addition, the system analyzes additional statistical parameters (for example, the average and standard deviation). These parameters serve as indications of restlessness or subject agitation and are presented to a clinician to support medical decision making. They may also be used as additional clinical parameters for baselining and scoring purposes.

In an embodiment of the present invention, system 10 calculates respiration rates and heart rates based on frequency domain analysis. For example, for the heart rate, signals in the frequency domain are often seen as a basic peak at the heart rate and additional peaks at whole number multiples of that basic frequency that represent the harmonics of the basic signal. In some cases, the peak in the spectral domain that corresponds to the heart rate is surrounded by other peaks of similar size so it is difficult to identify the one corresponding to the heart rate. In an embodiment, the signal processing unit identifies potential peaks representing the heart beat basic harmony and then adds to these peaks a measure based on the amplitude based on the relative height of the harmonic peaks before making the decision which peak corresponds to the subject's heart rate.

In an embodiment of the present invention, system 10 detects of heart rate using high frequency components of the spectrum using demodulation that uses a bank of band pass filters. For example, such a bank filter may include filters from 3 Hz up to 12 Hz, and each filter may be 1 Hz broad and have 0.5 Hz overlap with another filter. The algorithm selects the filter with the highest signal-to-noise ratio (SNR) of the heartbeat peak, and the system uses this filter until there is a change in subject's position, or to until large body motion is detected. (In clinical trials carried out by the inventors, it was found that the optimal filter can change by 4-5 Hz for the same subject in different positions.) For some applications, the SNR of the heartbeat peak is defined as the magnitude of this peak divided by its close neighborhood not including any whole number harmonics of the peak. If the frequency of the heart rate peak is f and the amplitude of the spectrum at frequency f is H(f), then:

$$SNR = \frac{H ( f )}{\begin{matrix}1 / 2 * ( mean ( H ( f - 0.5 f : f - 0.1 f ) + \\ mean ( H ( f + 0.1 f : f + 0.5 f )\end{matrix}} \qquad ( Equation 20 )$$

In an embodiment of the present invention, the system identifies the heart-beat-related signal by running a relatively high bandwidth band pass filter on the signal detected by a piezoelectric vibration sensor. The bandpass filter used has a passband of, for example, 30 Hz to 80 Hz. The resulting signal is run through a peak detection algorithm in order to identify the locations of the actual heart beats.

In an embodiment of the present invention, system 10 calculates a clinical parameter as defined hereinabove, such as respiration rate and/or heart rate, and records the results. The system subsequently calculates a representative value for the data for a specific period of time. Typically, the system calculates an average or median for the data for the period of time, or calculates a series of representative values for the data during smaller sub-periods of the period, and passes this series of values through a low pass filter or a median filter. The system generates an alert upon the onset of at least one of the following alert conditions (the system allows a clinician to set a level for each of the thresholds and timing ranges; alternatively, the system learns the parameter distribution for a specific subject, disease type, or hospital ward and sets the levels accordingly):
The representative value of the clinical parameter for a time period of between about 10 seconds and about 3 minutes, for example, for example, about 30 seconds, is greater than or less than a predefined threshold.
A sharp change occurs in the representative value of the clinical parameter for a time period of between about 10 seconds and about 3 minutes, for example, about 30 seconds. For example, a sharp change may be defined as at least a percentage change versus baseline of between about 20% and about 70%, for example, about 50%. The change is calculated versus the baseline, which is defined, for example, as the representative value for the clinical parameter for a certain amount of trailing time, e.g., the previous 15 minutes.
The clinical parameter shows a slow but substantial change. For example, the representative value of the clinical parameter measured in the most recent 10 minutes (A₁₀) may be compared to the representative value of the clinical parameter measured in the following time segments:
   Last hour (H₁)
   The hour before the last hour (H₂)
   The hour before the two last hours (H₃)
   The hour before the last three last hours (H₄)
A threshold is set between about 20% and about 70%, for example about 50%. The system generates an alarm if the following criterion is true:

Δᵢ=ABS[(A₁₀−Hᵢ)/A₁₀]; (Equation 21)

Alarm on=If [Max {Δ₁,Δ₂,Δ₃,Δ₄}>the threshold (e.g., 50%)] (Equation 22)
If a sudden loss in clinical parameter sensing is detected by the system without a change in weight (i.e., no bed exit has occurred), the system activates the alarm immediately.
The representative value of the clinical parameter during a most recent period of time, e.g., in the past 5 minutes, is different from the representative value for the clinical parameter during a substantially longer previous period of time, for example, the last 6 hours, by more than a certain number (e.g., 3) times the standard deviations of the clinical parameter within the substantially longer period of time. The range of 3 times the standard deviation around the representative value is defined as the accepted range for the clinical parameter.

In an embodiment of the present invention, system 10 identifies a slow change pattern and is configured with a threshold indicating when the system should generate an alert. The system calculates and outputs the amount of time until the subject will reach the alert threshold if the current slow trend continues. For example, if the system identifies a trend for an increase in breathing rate of 3 breaths/minute every hour and the current breathing rate is 21 breaths/minute and the threshold is 36, then the system calculates that the time to alert is 5 hours (5=(36−21)/3) and displays that value on the screen. This alert enables the clinician to evaluate the risk level of the current condition based on both the current value and the slow trend. In addition, in an embodiment, the system outputs a warning if the time to alert is below a threshold value. For example, if the time to alert is less than 2 hours, the system may display a warning message on the screen. For some applications, the system combines the current value of the reading and the slow trend into a single indication and/or warning decisions.

In an embodiment of the present invention, system 10 combines two or more changes in clinical parameters. For example, the system may sum the percentage change in representative value of the heart rate and respiration rate over the last 10 minutes, and compare the sum to a threshold. The system generates an alarm upon finding that the sum is greater than the threshold.

In an embodiment of the present invention, triggers for an alarm include events that combine heart and respiration deterioration. For example, the system generates an alarm upon find that both (a) respiration rate values are greater than a threshold value continuously over a period of time, e.g., between about 10 seconds and about 3 minutes, and (b) the heart rate values are greater than a threshold value continuously during the period. For some applications, the system generates the alarm if both conditions (a) and (b) are true for a period of time that is between about 10 seconds and about 3 minutes, for example about 30 seconds.

In an embodiment of the present invention, system 10 identifies a high level of variability of the subject's heart rate as an indication of a possible risk of arrhythmia. For some applications, system 10 filters out measured heart rates that are highly variable when these measured heart rates correlate with a high or highly variable level of body movement, as measured with a motion sensor, because the variability of these measured heart rates may have been caused by a change in heart rate caused by the subject's body motion.

In an embodiment of the present invention, the system assigns each clinical parameter measurement (e.g., respiratory rate) a confidence level as a function, for example, of the following: signal quality, signal to noise ratio, repeatability of the results of the clinical parameter measurement within very short time windows, and/or repeatability of the results using different sensors or different calculation algorithms (e.g., one in the frequency domain and another in the time domain). The system typically continuously updates the confidence levels. The system generates an alarm only if the confidence level of the activating clinical parameter is greater than a threshold. Alternatively, the system generates the alarm if the average confidence level for the clinical parameter over a period of time, e.g., between about 10 seconds and about 3 minutes is greater than a threshold level.

In an embodiment of the present invention, the system monitors a subject during time periods when he is awake and during time period when he is asleep. The variation in clinical parameters is in some cases lower during sleep than during wake periods. In an embodiment, the system uses different thresholds for identification of subject deterioration for the two different states. The system switches between these two levels of thresholds either automatically or manually. For example, a healthcare worker or caregiver may manually switch between sleep mode and wake mode upon observing when the subject changes wake state, by entering the change in state into system 10 via user interface 24. Alternatively or additionally, the system may automatically switch according to the time of day when subject is expected to be asleep or awake, or based on detection by the system whether the subject is awake or asleep, such as by detecting when the patient exhibits a high level of non-respiratory body movements vs. low levels of non-respiratory body movements as described hereinabove regarding techniques for identifying large body movement.

For example, a subject whose baseline breathing rate is 14 breaths/minute (bpm) may have alert activation thresholds set at 8 bpm and 30 bpm during wake period, but during sleep the range is narrowed to 8 bpm and 20 bpm, for more effective identification of deterioration. The use of the narrower threshold range during the wake state might create an unacceptable level of false alarms, but during sleep these tighter thresholds in some cases enable better identification of subject deterioration with few additional false alarms.

In an embodiment of the present invention, the system identifies during sleep when a subject is entering REM sleep phase as described hereinabove. Because the subject is expected to have a relatively high level of variability of certain clinical parameters during this REM phase, a higher level of variation threshold is set in order to prevent false alarm.

In an embodiment of the present invention, system 10 switches between two levels of thresholds according to the subject's level of restlessness, regardless of whether the subject is asleep.

In an embodiment of the present invention, the system uses more than two thresholds, and calculates the thresholds as a continuous function of the level of subject's activity or restlessness.

In an embodiment of the present invention, system 10 uses techniques for modifying thresholds for one or more of the alert conditions that are similar to techniques described hereinabove for adapting thresholds based on the level of activity/restlessness of the subject.

In an embodiment of the present invention, system 10 switches between different algorithms for calculating respiratory rates or heart rates between sleep and wake mode, and/or between low activity level and high activity level. For example, for some applications, it is more effective to use a time domain algorithm for calculating respiratory rate when the subject is awake and a frequency domain algorithm when the subject is asleep. Alternatively, the system switches between the different algorithms according to a level of subject activity and/or restlessness. For some applications, upon identifying that a subject is sleeping or in quiet rest, the system activates an early warning mechanism that generates an alert if these is a high risk that the subject will attempt to leave the bed. For example, if the subject is lying quietly in bed and the system suddenly identifies that the subject is moving around in bed for continuously for over 30 seconds, the system may generate an alert a clinician that the subject is at high risk of trying to exit the bed. This is useful for preventing subject falls, especially for elderly, demented subjects. For some applications, system 10 build a baseline of the subject's body movements during sleep and generates an alert upon detecting a movement pattern that is significantly different from baseline, which may indicate that the subject is having trouble sleeping or is transitioning out of sleep. For some applications, the system uses different criteria for generating alerts upon subject movement for different hours of the day. For example, between 2:00 AM and 5:00 AM a relatively low level of motion in a 30 second interval creates an alert, while at other times of the day the threshold is greater.

In an embodiment of the present invention, system 10 is configured to receive, for each of a plurality of wake states, respective specified ranges of values for a clinical parameter, such as heart rate or respiration rate. The system determines that the subject is in one of the wake states, such as using techniques described hereinabove. Responsively to a signal generated by motion sensor 30, the system calculates a representative value of the clinical parameter of the subject. The system generates an alert if the representative value falls outside the one of the specified ranges corresponding to the one of the wake states of the subject. Typically, the wake states include a sleep state and an awake state, or the wake states include an REM sleep state, a non-REM sleep state, and an awake state. For some applications, this technique is used to monitor subjects having a condition other than apnea or SIDS.

In some cases, movement of the subject reduces the accuracy of the detected parameters (e.g., respiratory rate and heart rate by a contactless sensor, and blood oxygen saturation and blood pressure by a contact sensor). In an embodiment of the present invention, system 10, when calculating the level of confidence given to the measurement, takes into account the level of subject's motion (restlessness) during the time of measurement. For some applications, if a value of a clinical parameter indicates that the system should generate an alarm, the system delays generating the alarm if the confidence level is lower. During this delay, the system continues to measure the clinical parameter and to evaluate whether to generate an alarm. If the value of the parameter throughout the delay, or on average during the delay, continues to indicate that an alarm is warranted, the system generates the alarm upon the conclusion of the delay. Thus, for example, assume that the system is configured to measure blood oxygen saturation, and to generate an alarm upon detecting that saturation drops below 90%. If the system identifies such a drop and does not detect any large body motion during the saturation measurement, the system generates an alert immediately. If, on the other hand, the system identifies such a drop and detects large body motion during the saturation measurement, the system continues to measure and average the saturation level during a delay, e.g., having a duration of 60 seconds, and generates an alarm only if the average over the full delay is below 90%. This technique generally reduces false alarms caused by motion artifacts.

In some cases, a change in a clinical parameter may be caused by large body motion of the subject. For example, a sudden increase in a subject's respiratory rate may be cause for alarm if the patient is lying still, but may be normal if the subject just exhibited restlessness in bed (this is particularly true for highly obese subjects). In an embodiment of the present invention, system 10 uses a tighter threshold or a quicker alert response time for changes in clinical parameters that do not occur immediately after or during a period of restlessness, and a second looser threshold for changes that occur immediately after or during a period of restlessness and that are to be expected to occur during restlessness (e.g., an increase in respiratory rate). For some applications, the system does not implement this double threshold if the restlessness occurs after the identification of the change in the clinical parameter.

In an embodiment of the present invention, upon identifying that a clinical parameter is greater than a threshold for generating an alert, the system delays generating the alert for a certain period of time. For example, the delay period may have a duration of between about 15 seconds and about 10 minutes, depending on clinician input, prior variability of the subject's readings, a confidence level of the measurement, and the subject's current condition (e.g. asleep, awake, REM sleep, known asthma condition, etc.). During this delay period the system further verifies that the reading was indeed accurate and/or is consistently beyond the alert threshold. Upon such verification, the system generates the alert. Otherwise the system does not generate the alert. This technique helps prevent false alerts.

In an embodiment of the present invention, system 10 identifies the onset and monitors the progression of sepsis according to changes in clinical parameters of a subject, for example, in heart rate and/or respiration rate of the subject. For some applications, the system identifies sepsis responsively to detection of an increase in a level of tremor, and/or. For some applications, the system identifies sepsis responsively to detection of rapid shallow breaths, characterized by a decrease in the magnitude of the breathing-related motion together with an increase in the respiration rate. For some applications, the system calculates a sepsis score based on the combination of two or more of the following parameters: respiration rate, respiration depth (shallow vs. deep), heart rate, and tremor. When the score changes significantly versus baseline or crosses a predefined threshold, the system generates an alert for a clinician.

In an embodiment of the present invention, system 10 identifies rapid shallow breaths by identifying an increase in respiration rate with a decrease in respiration motion signal size and without a change in subject's posture compared to before the onset of shallow breathing.

In an embodiment of the present invention, system 10 identifies rapid shallow breathing by identifying a decrease in magnitude of respiratory sinus arrhythmia.

In an embodiment of the present invention, system 10 notifies the nursing care staff of the any of the alarm conditions described herein using the existing nurse call system used in the healthcare facility.

In an embodiment of the present invention, system 10 persistently reminds nurses of a continued deterioration in the condition of a subject until intervention is successful.

In an embodiment of the present invention, system 10 identifies the entry of subject 12 into bed, such as using techniques described hereinabove. For some subjects it is important that the subject not spend too much time in bed without exiting the bed (for example, in order to prevent pressure sores, e.g. bed sores). System 10 alerts the medical staff if the subject has not left the bed for a predefined period of time, for example, 12 hours. For some applications, system 10 also identifies that a subject has changed position in bed or has been turned over, such as using techniques described hereinabove. Alternatively or additionally, the system identifies posture change using techniques described in U.S. patent application Ser. No. 11/552,872, which published as US Patent Application Publication 2007/0118054 to Pinhas et al., and which is assigned to the assignee of the present application and incorporated herein by reference. The system generates an alert if the subject has not changed position in bed or was not turned over for a predefined period of time. For some applications, system 10 comprises a user interface that enables the clinician to indicate to the system that the subject has been turned over in bed. This log enables historical analysis and creates a record that proper treatment has been provided to the subject. The system's automatic detection of subject motion is implemented either to confirm the clinician's entry or to replace it. For some applications, the system uses manual indication of subject turning over to calibrate the automatic posture change detection algorithm.

In an embodiment of the present invention, system 10 calculates a score based on the level of motion and number of subject posture changes. The system analyzes this score over a time period ranging from about 15 minutes to about 3 days, for example about 4 hours. This score serves as an indication of the level of risk of development of a pressure ulcer. This score index may be adapted according to the guidelines set by relevant regulatory bodies or by an attending physician. For example, most hospitals have a policy that requires subjects who are at risk of developing pressure sores (e.g., bed sores) be turned over or repositioned at least once every two hours.

In accordance with a first exemplary technique for calculating this score, the system uses the following equation:

Score=100−(TC/RTC)*100 (Equation 23)

wherein TC is the time from last posture change measured in minutes, and RTC is the recommended time in minutes between posture changes according to guidelines or physician order.

For some applications, the calculated score is displayed numerically and graphically, e.g., color-coded. For example, the score is shown as green if it is greater than 95. A score of 85-95 is shown as yellow, and a score below 85 is shown as red. For some applications, if the score falls below a threshold, the system generates an alarm in order to alert a clinician and enable timely intervention.

In accordance with a first exemplary technique for calculating this score, the system uses the following equation:

Score=100−(TC/RTC)*100+MPR (Equation 24)

wherein TC is time from last posture change measured in minutes, RTC is recommended time in minutes between posture changes according to guidelines or physician order, and MPR is percentage of time during the last hour in which the subject made large body movements (e.g., each 15 second interval is marked as movement if a large body movement is identified in the interval, and the percentage of such marked intervals during the last hour is used in Equation 24).

In an embodiment of the present invention, the system calculates an average score over a time period ranging from about one hour to the duration of the subject's stay in the hospital. The average score serves as an indication of the compliance (i.e., a compliance index) of the clinical team with the designated guideline. The average score can be used by the hospital administration in order to evaluate team performance and enable continuous improvement of subject care and subject experience.

In an embodiment of the present invention, this score also reflects changes in respiration rate, heart rate, and/or level of tremor compared to baseline. An increase in these parameters may indicate an infection that in some cases accompanies the onset of pressure sores, e.g., bed sores. For some applications, the score alternatively or additionally reflects a level of variability in the heart rate and respiration rate.

In an embodiment of the present invention, system 10 is used to identify when a subject is in bed. Periodically, e.g., every hour, the system logs whether or not there is a subject in the bed. For example, this logging may enable hospital equipment rental providers to charge hospitals for rental beds only for the days or hours when a subject uses the bed.

In an embodiment of the present invention, system 10 has a user interface that enables a clinician to enter data relating to subject care for logging together with the clinical parameters measured by the system. For example, the clinician may be able to enter into the system when a subject is fed, is administered medication, has his temperature read, or undergoes a procedure. Alternatively or additionally, system 10 interfaces with a hospitals computer system for access to such relevant data. System 10 generates reports indicating the changes in clinical parameters and the timing of any such events. Furthermore, for some applications, system 10 identifies patterns that indicate a correlation between events and changes in parameters. For example, if a rapid increase in breathing rate is identified in at least two events within 60 minutes of administration of medication, the system generates an alert for a clinician to evaluate whether a change in medication is required. Such an increase in breathing rate may indicate, for example, that the subject is allergic to the medication used.

In an embodiment of the present invention, system 10 is used to monitor a subject who has been severely burned such that sensors cannot be connected to his body.

In some of the applications assigned to the assignee of the present application and incorporated herein by reference, measurement of vibration data using a sensor installed under or within a bed mattress has been shown, to provide a high quality signal suitable for extraction of accurate heart and respiration rates. In some cases, unfavorable recording conditions are encountered, such as because of large body movements or other external perturbations.

In an embodiment of the present invention, system 10 reduces signal noise level using an adaptive noise cancellation technique. The basic concept of noise cancellation is to pass the noisy signal through a noise-suppression filter, which uses auxiliary information such as a reference noise channel for adaptive noise removal. Reference information is commonly obtained by using multiple sensors, where at least one primary sensor is positioned to capture the noise contaminated signal channel and at least one auxiliary sensor is positioned to measure the noise contribution.

In some cases, pure noise information is often unattainable, and suboptimal optimization approaches are used. In the case of a near signal source and a remote external noise source, in an embodiment of the present invention, the system amplifies the near field signal and suppresses the far field noise. Near field data is distinguished from far field data by using a pair of closely located identical sensors. Far field signals are received equally in both sensors, while near field signals are received differently. Thus, taking the difference signal between the two sensors cancels out far field data while retaining near field information. In an embodiment of the present invention, multiple sensors are used to optimize noise elimination by selecting the sensors with the most similar signal.

In an embodiment of the present invention, the sensor plate holds several sensors at different orientations, in order to obtain primary and auxiliary signals using a compact sensing structure. This measures different projections of the signal and noise vectors, thereby providing the means to enhance the signal and suppress the noise.

In an embodiment of the present invention, the compact sensing structure comprises three sensor units arranged to form a pyramid-like structure, allowing reception of signal and noise components from all directions.

In an embodiment of the present invention, sensor arrangements are used to provide information regarding a plurality of angles and/or about more than three directions, facilitating optimized signal restoration using optimization schemes such as mean least-square analysis.

In an embodiment of the present invention, the system comprises directional sensors to enhance the signal coming from the allowed reception zone and suppress signals from other directions, thereby increasing separability of signal and noise contributions.

In an embodiment of the present invention, two identical sensors are placed in close proximity to one another and oriented in the same orientation, such that the difference signal between the two sensors enhances near field data and suppresses far field interference. In the case of non-ideal sensors, the system may use adaptive subtraction.

The following examples illustrate three schemes for signal enhancement. For simplicity, the examples relate to two-dimensional analysis; however, expansion from two to three dimensions is straightforward to those skilled in the art who have read the present patent application. The first two examples use two perpendicular sensors.

### Example 1

Sensor A receives a compound signal comprised of a superposition of a signal s(t) and noise e(t): x(t)=s(t)+e(t).

Sensor B receives a projection of the noise denoted e′(t).

For this example, assume that Signal s(t) and noise e(t) are uncorrelated. The signal s(t) is extracted via adaptive elimination of a reconstructed noise signal from the compound signal plus noise x(t) received by sensor A, by minimizing the mean-square difference: MIN {[[s(t)+e(t)]−h(t)*e′(t)]̂2}, wherein h(t) denotes the impulse response of a linear time-invariant (LTI) filter.

Solving for h(t) yields the desired solution: s(t)=x(t)−h(t)*e′(t).

### Example 2

Sensors A and B receive different projections of a compound signal comprised of a superposition of a signal s(t) and noise e(t). For this example, assume that:

signal x(t) and noise e(t) are uncorrelated; and

signal and/or noise spectrum are known.

The axes are rotated to enhance signal and/or noise projections, until the desired characteristic spectrum is achieved, as follows (alpha and beta are incidence angles of the signal and noise, respectively):

Sensor A reads: S1(t)=x(t)*sin(alpha)+e(t)*sin(beta)

Sensor B reads: S2(t)=x(t)*cos(alpha)+e(t)*cos(beta)

The axes are rotated by gamma degrees, yielding:

S1′(t)=S1(t)*cos(gamma)+S2(t)*sin(gamma)

S2′(t)=S1(t)*sin(gamma)+S2(t)*cos(gamma)

The rotated signals S1′(t) and S2′(t) are calculated for all angles until noise contribution is cancelled (when gamma=pi-beta), and a scaled version of the desired signal is obtained:

$$\begin{matrix}S {1}^{′} ( t ) = [ x ( t ) * sin ( alpha ) + e ( t ) * sin ( beta ) ] * cos ( gamma ) + \\ [ x ( t ) * cos ( alpha ) + e ( t ) * cos ( beta ) ] * sin ( gamma ) \\ = [ x ( t ) * sin ( alpha ) + e ( t ) * sin ( beta ) ] * cos ( pi - beta ) + \\ [ x ( t ) * cos ( alpha ) + e ( t ) * cos ( beta ) ] * sin ( pi - beta ) \\ = x ( t ) * [ sin ( alpha ) ) cos ( pi - beta ) + cos ( alpha ) * sin ( pi - beta ) ] + \\ e ( t ) * [ sin ( beta ) * cos ( pi - beta ) + cos ( beta ) * sin ( pi - beta ) ] \\ = x ( t ) * sin ( pi + alpha - beta ) + e ( t ) * sin ( pi ) \\ = x ( t ) * sin ( beta - alpha )\end{matrix}$$

### Example 3

Identical sensors A and B are placed in close proximity and at the same orientation. Both sensors receive a superposition of near field signals and far field noise.

For this example, assume that:
the distance between the sensors is significantly smaller than their distance from the noise source, but is of the order of magnitude of the distance from the signal source; and
the signal source is comprised of a superposition of at least two differently oriented signal sources. For simplicity, the following description assumes two signal sources.

Let x1(t) and x2(t) denote the two near field signal sources.

Let e(t) denote the far field noise signal.

Sensor A reads: S1(t)=x1(t)+e(t)

Sensor B reads: S2(t)=x2(t)+e(t)

Then the difference signal is:

$$\begin{matrix}Sdiff = S 1 ( t ) + S 2 ( t ) \\ = x 1 ( t ) - x 2 ( t ) + e ( t ) - e ( t ) \\ = X 1 ( t ) - x 2 ( t )\end{matrix}$$

Thus, the far field signal is suppressed.

Although some embodiments described herein relate specifically to asthmatic episodes or CHF, the principles of the present invention may be applied, mutatis mutandis, to predicting and monitoring of one or more other respiratory and non-respiratory conditions that affect normal health patterns.

Techniques described herein may be practiced in combination with techniques described in one or more of the following applications, which are assigned to the assignee of the present patent application and are incorporated herein by reference. In an embodiment, techniques and apparatus described in one or more of the following applications are combined with techniques and apparatus described herein:
U.S. Provisional Patent Application 60/674,382;
U.S. Provisional Patent Application 60/692,105;
U.S. Provisional Patent Application 60/731,934;
U.S. Provisional Patent Application 60/784,799;
U.S. Provisional Patent Application 60/843,672;
U.S. Provisional Patent Application 60/924,459, filed May 16, 2007;
U.S. Provisional Patent Application 60/924,181, filed May 2, 2007;
U.S. Provisional Patent Application 60/935,194, filed Jul. 31, 2007;
U.S. Provisional Patent Application 60/981,525, filed Oct. 22, 2007;
U.S. Provisional Patent Application 60/983,945, filed Oct. 31, 2007;
U.S. Provisional Patent Application 60/989,942, filed Nov. 25, 2007;
U.S. Provisional Patent Application 61/028,551, filed Feb. 14, 2008;
U.S. Provisional Patent Application 61/034,165, filed Mar. 6, 2008;
U.S. patent application Ser. No. 11/197,786, filed Aug. 3, 2005, which issued as U.S. Pat. No. 7,314,451;
U.S. patent application Ser. No. 11/782,750;
U.S. patent application Ser. No. 11/446,281;
U.S. patent application Ser. No. 11/755,066;
U.S. patent application Ser. No. 11/048,100, filed Jan. 31, 2005, which issued as U.S. Pat. No. 7,077,810;
International Patent Application PCT/IL2005/000113, which published as WO 2005/074361;
International Patent Application PCT/IL2006/000727, which published as WO 2006/137067; and
International Patent Application PCT/IB2006/002998, which published as WO 2007/052108.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-75. (canceled)

76. Apparatus for use during endotracheal intubation of a subject, the apparatus comprising:
at least one sensor configured to sense motion of the subject, and generate a signal responsively thereto;
an output unit; and
a control unit, configured to:
detect an aspect of the intubation by analyzing a component of the signal having a frequency of less than 20 Hz, and
drive the output unit to generate an output indicative of the aspect of the intubation.

77. The apparatus according to claim 76, wherein the sensor is configured to be coupled to an external surface of a body of the subject.

78. The apparatus according to claim 77, wherein the sensor comprises first and second sensors that are configured to be coupled to the external surface in respective vicinities of a left lung and a right lung of the subject, and to generate respective first and second signals responsively to the respective motion in the vicinities of the left and right lungs.

79. The apparatus according to claim 78, wherein the control unit is configured to detect the aspect of the intubation upon finding that the first and second signals have different strengths.

80. The apparatus according to claim 76, wherein the control unit is configured to detect the aspect of intubation by detecting malpositioning of a tube used for the intubation.

81. The apparatus according to claim 76, wherein the control unit is configured to analyze the component of the signal during performance of the intubation.

82. The apparatus according to claim 76, wherein the output is audible, and wherein the output unit is configured to generate the audible output.

83. The apparatus according to claim 76, wherein the control unit is configured to identify a difference in ventilation effectiveness of two lungs of the subject.

84. The apparatus according to claim 76, wherein the control unit is configured to detect the aspect of the intubation by detecting insertion of a tube used for the intubation into an esophagus of the subject.

85. A method comprising:
sensing motion of an endotracheally-intubated subject, and generating a signal responsively thereto;
detecting an aspect of the intubation by analyzing a component of the signal having a frequency of less than 20 Hz; and
in response thereto, generating an output indicative of the aspect of the intubation.

86. The method according to claim 85, wherein sensing comprises sensing via a sensor coupled to an external surface of a body of the subject.

87. The method according to claim 86, wherein sensing comprises sensing via at least two sensors coupled to the external surface.

88. The method according to claim 87, wherein sensing comprises sensing via first and second ones of the sensors that are coupled to the external surface in respective vicinities of a left lung and a right lung of the subject, and generating respective first and second signals with the first and second sensors responsively to the respective motion in the vicinities of the left and right lungs.

89. The method according to claim 88, wherein detecting the aspect of the intubation comprises detecting the aspect of the intubation upon finding that the first and second signals have different strengths.

90. The method according to claim 85, wherein detecting the aspect of the intubation includes detecting malpositioning of a tube used for the intubation.

91. The method according to claim 85, wherein sensing comprises sensing the motion while the intubation is being performed.

92. The method according to claim 85, wherein generating the output comprises generating an audible output.

93. The method according to claim 85, wherein determining the aspect of the intubation comprises using a plurality of sensors to identify a difference in ventilation effectiveness of two lungs of the subject.

94. The method according to claim 85, wherein detecting the aspect of the intubation comprises detecting insertion of a tube used for the intubation into an esophagus of the subject.

95-160. (canceled)

161. The apparatus according to claim 76, wherein the control unit is configured to detect ventilation-related vibrations of the subject by analyzing the component of the signal having a frequency of less than 20 Hz.

162. The method according to claim 85, wherein analyzing a component of the signal having a frequency of less than 20 Hz comprises detecting ventilation-related vibrations of the subject.
